SEQUENCE LISTING (4 sequences)

SEQ ID NO: 1 - DNA, 57 bp, Artificial Sequence
  Description of Artificial Sequence Synthetic primer
aagcagtggtatcaacgcagagtacttttttttttttttttttttttttttttttvn

SEQ ID NO: 2 - DNA, 30 bp, Artificial Sequence
  Description of Artificial Sequence Synthetic oligonucleotide
aagcagtggtatcaacgcagagtacatggg

SEQ ID NO: 3 - DNA, 23 bp, Artificial Sequence
  Description of Artificial Sequence Synthetic primer
aagcagtggtatcaacgcagagt

SEQ ID NO: 4 - DNA, 15 bp, Artificial Sequence
  Description of Artificial Sequence Synthetic oligonucleotide
acagtcgatagatag

(19) United States
(12) Patent Application Publication
(10) Pub. No.: US 2016/0186262 A1
(43) Pub. Date: Jun. 30, 2016

(54) COMPOSITIONS AND METHODS FOR GENETIC ANALYSIS OF EMBRYOS

(71) Applicant: REPRODUCTIVE GENETICS AND TECHNOLOGY SOLUTIONS, LLC, Los Angeles, CA (US)

(72) Inventor: Mark T. JOHNSON, Los Angeles, CA (US)

(21) Appl. No.: 14/763,068

(22) PCT Filed: Jan. 23, 2014
(86) PCT No.: PCT/US14/12833

§ 371 Date: Jul. 23, 2015

Related U.S. Application Data
(60) Provisional application No. 61/755,760, filed on Jan. 23, 2013.
(60) Provisional application No. 61/785,752, filed on Mar. 14, 2013.

(51) Int. Cl. C12Q 1/68

(52) U.S. Cl. CPC C12Q 1/6883; C12Q 2600/156

(57) ABSTRACT

This disclosure provides compositions and methods for determining a presence or absence of a genomic copy number alteration (CNA) in an embryo, wherein the method comprises analysis of RNA from an embryo or cDNA derived from this RNA. Generally, the compositions and methods provide for the acquisition of a sample containing RNA produced by an embryo, application of one or more of at least 3 different methods for detecting CNAs. One method can identify CNAs based on the identification of alterations in expression of loci or alleles affected by the CNA. Another can identify CNAs based on the identification of associated breakpoint. A third can identify CNAs based on expression profiles that are associated with CNAs. A variety of other genetic and biologic analyses can be performed on the RNA in combination with the copy number analyses. Analysis of copy number in embryos can provide information that can provide important clinical information pertaining to the health and developmental potential of an embryo that can impact the plans of the parents and clinical staff for the embryo.

### CROSS-REFERENCE

This application claims the benefit of U.S. Patent Application No. 61/755,760, filed Jan. 23, 2013, and 61/785,752, filed Mar. 14, 2013, which applications are herein incorporated by reference in their entireties.

### SEQUENCE LISTING

The instant application contains a Sequence Listing which has been submitted electronically in ASCII format and is hereby incorporated by reference in its entirety. Said ASCII copy, created on Sep. 25, 2015, is named 44047-701.831_SL.txt and is 1,535 bytes in size.

### BACKGROUND OF THE DISCLOSURE

Human embryos, including those generated through assisted reproductive technologies (ART) can be prone to various genetic alterations, including abnormalities in the number of copies of segments of their genomes. Recent studies have shown that a substantial proportion of human embryos generated in vitro through ART contain at least some cells with genomic copy number abnormalities (CNAs) that involve entire or large segments of chromosomes when evaluated during the preimplantation period, the period that extends from conception until the embryo implants into the uterine wall. These large CNAs cannot be attributed solely to advanced age or impaired fertility of gamete donors as there is also a high rate of these genetic abnormalities in embryos generated by ART using sperm and egg from young donors without history of infertility. These large CNAs arise as a result of errors in the meiotic divisions of the gamete(s) and/or the mitotic divisions of the early embryo and frequently have a negative impact on the health and/or development of conceptuses. There is need in the art for improved screening methods for detection of CNAs in preimplantation embryos, especially ones derived from ART.

### SUMMARY OF THE DISCLOSURE

In one aspect, a method of determining a presence or absence of a genomic copy number alteration in a preimplantation embryo is provided, the method comprising analyzing RNA from the preimplantation embryo, or cDNA generated from RNA from the preimplantation embryo, to determine the presence or absence of the genomic copy number alteration in the preimplantation embryo. In some cases, the cDNA is generated by reverse transcribing RNA from the preimplantation embryo. In some cases the analyzing comprises generating sequence data for the RNA or the cDNA. In some cases, the generating sequence data comprises high-throughput sequencing. In some cases, the generating sequence data comprises whole transcriptome sequencing. In some cases, the generating sequence data comprises partial transcriptome sequencing. In some cases, the analyzing comprises aligning the sequence data to a reference genome or reference transcriptome. In some cases, the analyzing comprises quantitating the sequence data. In some cases, the analyzing comprises performing an algorithm on the sequence data.

In some cases, the sequence data comprises sequence reads. In some cases, the analyzing comprises comparing an abundance of the sequence reads corresponding to one or more regions on a first chromosome to an abundance of sequence reads corresponding to one or more regions on a second chromosome. In some cases, the abundance of the sequence reads corresponding to one or more regions on a first chromosome is normalized to a number of the sequence reads corresponding to one or more regions on a second chromosome. In some cases, the abundance of the sequences reads corresponding to one or more regions on a first chromosome is normalized to an abundance of the sequence reads corresponding to regions on a plurality of chromosomes. In some cases, the analyzing comprises comparing an abundance of sequence reads corresponding to one or more regions from a plurality of chromosomes to an abundance of sequence reads corresponding to one or more regions on a second chromosome. In some cases, the first and second chromosomes are from the same cell or same embryo. In some cases, the first and second chromosomes are from different cells or different embryos.

In some cases, the copy number state of the second chromosome is known. In some cases, the copy number state of the second chromosome is not known. In some cases, the second chromosome is suspected of having a normal copy number.

In some cases, the analyzing comprises normalizing an abundance of the sequence reads corresponding to one or more regions on a first chromosome to generate a normalized chromosome count, and comparing the normalized chromosome count to a normalized chromosome count for a reference sample from one or more embryos. In some cases, the one or more regions are selected from the group consisting of: an exon, a gene, an allele, a locus, genome, a genome coordinate, a transcriptional unit or a region of defined length of the transcriptome.

In some cases, the high-throughput sequencing comprises a) bridge amplification and incorporation of four fluorescently-labeled, reversible terminator-bound dNTPs; b) measurement of release of inorganic phosphate; c) passing the cDNA through a nanopore; or d) measuring hydrogen ion release during polymerization.

In some cases, the analyzing comprises hybridizing the RNA or cDNA to one or more probes. In some cases, the one or more probes are part of a microarray.

In some cases, the analyzing comprises amplifying the RNA or cDNA. In some cases, the amplifying comprises in vitro RNA synthesis. In some cases, the amplifying comprises amplification of selected RNAs or cDNAs. In some cases, the amplifying comprises amplification of random RNAs or cDNAs. In some cases, the amplifying comprises performing a polymerase chain reaction (PCR) on the cDNA. In some cases, the PCR is real-time PCR.

In some cases, the amplifying comprises isothermal amplification. In some cases, the amplifying comprises linear amplification. In some cases, the amplifying comprises isothermal linear amplification.

In some cases, the RNA is from a plurality of preimplantation embryos, or the cDNA generated from RNA from a plurality of preimplantation embryos. In some cases, the RNA from each of the plurality of preimplantation embryos is indexed, or the cDNA generated from RNA from each of the plurality of preimplantation embryos is indexed.

In some cases, the indexing comprises tagging each RNA or cDNA with a barcode.

In some cases, the analyzing comprises annealing a plurality of probe-pairs to a plurality of individual RNA or cDNA molecules. In some cases, each probe-pair comprises a capture probe capable of annealing to an individual RNA or cDNA and a reporter probe capable of annealing to the individual RNA or cDNA.

In some cases, the analyzing comprises comparing an amount of RNA or cDNA derived from one or more regions to an amount of RNA or cDNA derived from the one or more regions from one or more embryos of known copy number for the one or more regions.

In some cases, the analyzing comprises comparing an amount of RNA or cDNA derived from one or more regions to a median value of RNA or cDNA derived from the one or more regions from one or more embryos of known copy number for the one or more regions. In some cases, the analyzing comprises comparing an amount of RNA or cDNA derived from one or more regions to a median expression value. In some cases, the analyzing comprises comparing an amount of RNA or cDNA derived from one or more regions to a model. In some cases, the analyzing comprises comparing an amount of RNA or cDNA derived from one or more regions to a distribution value. In some cases, the analyzing comprises comparing an amount of RNA or cDNA derived from one or more regions to a median expression value of RNA or cDNA derived from the one or more regions from a plurality of embryos. In some cases, the analyzing comprises comparing a normalized expression value for RNA or cDNA derived from one or more regions to an amount of RNA or cDNA derived from the one or more regions of known copy number from one or more embryos. In some cases, the analyzing comprises comparing a normalized expression value for RNA or cDNA derived from one or more regions to a median value of RNA or cDNA derived from the one or more regions of known copy number from one or more embryos. In some cases, the analyzing comprises comparing a normalized expression value for RNA or cDNA derived from one or more regions to a median expression value of RNA or cDNA derived from the one or more regions from a plurality of embryos.

In some cases, the analyzing comprises determining a first ratio of an amount of RNA or cDNA derived from a first set of one or more regions to an amount of RNA or cDNA derived from a second set of one or more regions, and comparing the first ratio to a second ratio derived from one or more embryos, wherein the second ratio is a ratio of an amount of RNA or cDNA derived from the first set of one or more regions to an amount of RNA or cDNA derived the second set of one or more regions.

In some cases, the analyzing comprises determining a first ratio of an amount of RNA or cDNA derived from a first set of one or more regions to an amount of RNA or cDNA derived from a second set of one or more regions, and comparing the first ratio to a second ratio derived from a plurality of embryos, wherein the second ratio is a ratio of an amount of RNA or cDNA derived from the first set of one or more regions to an amount of RNA or cDNA derived from the second set of the one or more regions.

In some cases, the analyzing comprises comparing an amount of RNA or cDNA derived from one allele corresponding to one or more regions on a chromosome to an amount of RNA or cDNA derived from another allele corresponding to the one or more regions on the chromosome to determine an allele ratio, and comparing the allele ratio to a reference ratio of alleles to determine a presence or absence of a copy number alteration of one of the alleles. In some cases, the analyzing comprises comparing an amount of RNA or cDNA derived from one allele corresponding to one or more regions on a chromosome to an amount of RNA or cDNA derived from another allele of the same locus with known copy number status from one or more samples. In some cases, the analyzing comprises comparing an amount of RNA or cDNA derived from one allele corresponding to one or more regions on a chromosome to a median amount of the RNA or cDNA derived from the same allele from one or more samples with known copy number status of the allele. In some cases, the analyzing comprises determining a ratio of alleles of one or more regions, and comparing the ratio to a ratio of alleles of the one or more regions from one or more embryos with known copy number status of each allele. In some cases, the analyzing comprises determining a ratio of alleles of one or more regions, and comparing the ratio to a ratio of alleles of the one or more regions from a plurality of embryos. In some cases, the one or more regions are selected from the group consisting of: an exon, a gene, an allele, a locus, genome, a genome coordinate, a transcriptional unit or a region of defined length of the transcriptome. In some cases, the alleles are parental alleles.

In some cases, the determining the presence or absence of a copy number alteration comprises use of an algorithm. In some cases, the determining the presence or absence of a copy number alteration comprises performing a statistical analysis. In some cases, the analyzing comprises performing a haplotype analysis. In some cases, the copy number alteration is associated with a loss of heterozygosity.

In some cases, the analyzing comprises identifying one or more breakpoints associated with a copy number alteration. In some cases, the analyzing comprises identifying breakpoint sequence in massively parallel sequencing data by identifying split reads. In some cases, the analyzing comprises identifying breakpoint sequence in massively parallel sequencing data by identifying flanking sequences. In some cases, the flanking sequence identification comprises identifying discordant paired end reads.

In some cases, the RNA comprises transcribed RNA. In some cases, the transcribed RNA comprises messenger RNA. In some cases, the transcribed RNA comprises noncoding RNA. In some cases, the messenger RNA comprises a plurality of transcripts. In some cases, the plurality of transcripts comprises random transcripts.

In some cases, the method further comprises preparing a report based on the analyzing. In some cases, the method further comprises sending the report to a subject.

In some cases, a plurality of preimplantation embryos is analyzed. In some cases,

the preimplantation embryo is a mammalian preimplantation embryo. In some cases, the mammalian preimplantation embryo is a human preimplantation embryo. In some cases, the mammalian preimplantation embryo is from a domestic animal. In some cases, the mammalian preimplantation embryo is from an endangered animal.

In some cases, the method further comprises selecting the preimplantation embryo for transfer to a reproductive tract of a female based on the analyzing. In some cases, the method further comprises placing the selected preimplantation embryo in a reproductive tract of the female based on the analyzing. In some cases, the selected preimplantation embryo is at the blastocyst stage when the preimplantation embryo is placed in the reproductive tract of the female.

In some cases, the selecting comprises analyzing the morphology of the preimplantation embryo. In some cases, the selecting does not comprise analyzing the morphology of the preimplantation embryo. In some cases, the selecting comprises analyzing genomic DNA from the preimplantation embryo. In some cases, the selecting does not comprise analyzing genomic DNA from the preimplantation embryo.

In some cases, the method further comprises performing secretome and metabolic profiling of culture media in which the preimplantation embryo is cultured.

In some cases, the preimplantation embryo is generated from an oocyte from the female. In some cases, the preimplantation embryo is generated from an oocyte derived from ovarian tissue cultured in vitro. In some cases, the preimplantation embryo is generated from an oocyte derived from a germ cell in vitro. In some cases, the preimplantation embryo is generated from an oocyte derived from an ovarian tissue transplant. In some cases, the preimplantation embryo is generated from an oocyte derived from a stem cell. In some cases, the preimplantation embryo is generated from an oocyte from a second female, wherein the female receiving the preimplantation embryo and the second female are not the same female.

In some cases, the method further comprises cryopreserving the preimplantation embryo based on the analyzing.

In some cases, the preimplantation embryo is generated in vitro. In some cases, the preimplantation embryo is generated by in vitro fertilization. In some cases, the preimplantation embryo is generated by intracytoplasmic sperm injection. In some cases, the preimplantation embryo is generated in vitro from one or more oocytes derived from a female following stimulation of the female with exogenous hormones. In some cases, the preimplantation embryo is generated in vitro from one or more oocytes derived from a female who does not receive exogenous hormones. In some cases, the preimplantation embryo is in the preimplantation period. In some cases, the preimplantation period encompasses the period that begins with fertilization and extends to the latest timepoint at which an embryo can be maintained in vitro and still produce a healthy liveborn following transfer to the female. In some cases, the preimplantation embryo is at the blastocyst stage.

In some cases, determining a presence or absence of a copy number alteration in the preimplantation embryo correlates with preimplantation embryonic health or developmental potential.

In some cases, the determining the presence or absence of a copy number alteration comprises determining if the RNA has a pattern of expression associated with one or more copy number alterations. In some cases, the analyzing the RNA or cDNA comprises determining regional expression of the RNA or cDNA, identifying breakpoint sequence, and/or detecting a signature expression profile associated with a copy number alteration. In some cases, the method further comprises analyzing the epigenetic status of the genome of the preimplantation embryo.

In some cases, the method further comprises analyzing the RNA to determine a sex of the preimplantation embryo. In some cases, the sex is male. In some cases, the sex is female.

In some cases, the method further comprises analyzing the RNA or cDNA to determine expression patterns of regions associated with one or more responses to environmental stress. In some cases, the stress comprises exposure to a toxin, a mutagen, light, high or low temperature, high or low oxygen, oxidative stress, high or low osmolarity, mechanical insult, suboptimal culture conditions or inadequate nutrition. In some cases, the method further comprises analyzing the RNA or cDNA to determine expression patterns of regions associated with metabolism. In some cases, the method further comprises analyzing the RNA or cDNA to determine expression patterns of mitochondrial regions. In some cases, the method further comprises assessing mitochondrial load. In some cases, the method further comprises assessing metabolic activities.

In some cases, the analyzing comprises analyzing expression of one or more RNAs or cDNAs. In some cases, the analyzing comprises analyzing the expression of one or more genomic regions. In some cases, the analyzing comprises analyzing expression of one or more loci. In some cases, the analyzing comprises analyzing expression of one or more alleles. In some cases, an expression level of the one or more loci correlates with embryonic health or developmental potential of the preimplantation embryo.

In some cases, the method further comprises analyzing the RNA or cDNA to determine a presence or absence of one or more mutations in one or more loci. In some cases, the method further comprises performing linkage analysis.

In some cases, the copy number alteration is an aneuploidy. In some cases, the aneuploidy involves chromosome 13, 18, 21, X, or Y. In some cases, the aneuploidy is a trisomy. In some cases, the trisomy is trisomy 13, trisomy 18, or trisomy 21. In some cases, the trisomy is trisomy 21. In some cases, the aneuploidy comprises a portion of a chromosome. In some cases, the copy number alteration is a monosomy.

In some cases, the analyzing comprises use of an algorithm executed on a computer.

In some cases, the RNA comprises RNA derived from a subcellular compartment of the preimplantation embryo. In some cases, the subcellular compartment is a nucleus. In some cases, the subcellular compartment is cytoplasm. In some cases, the preimplantation embryo exists in a culture media, and the RNA is isolated from the culture media. In some cases, the embryo is mosaic for a copy number alteration.

In some cases, the determining the presence or absence of the genomic copy number alteration comprises determining an abundance of RNA or cDNA in one or more pre-defined regions of a transcriptome or genome to generate one or more regional expression counts. In some cases, the pre-defined region is selected from the group consisting of: an exon, a gene, an allele, a locus, a transcriptional unit or a region of defined length of the transcriptome or genome.

In some cases, the determining the presence or absence of the genomic copy number alteration in a sample comprises using one or more algorithms to compare one or more regional expression counts from a sample to a reference. In some cases, the determining the presence or absence of the genomic copy number alteration comprises comparing a regional expression count of one or more pre-defined regions in the RNA or cDNA to a reference to generate a relative regional expression value. In some cases, the reference comprises one or more regional expression counts. In some cases, the reference is generated from one preimplantation embryo. In some cases, the reference is generated from more than ten preimplantation embryos. In some cases, the reference is generated from more than 100 preimplantation embryos. In some cases, the reference is generated from more than 1000 preimplantation embryos. In some cases, the reference is generated from one or more preimplantation embryos, and wherein a genotype of the one or more preimplantation embryos is known. In some cases, the reference is generated from one or more preimplantation embryos, and wherein a genotype of the one or more preimplantation embryos is not known. In some cases, the reference region expression count comprises a mean, median, distribution, or model. In some cases, the reference comprises regional expression counts derived from one or more cells or embryos.

In some cases, the regional expression count is determined by sequencing. In some cases, the sequencing comprises generating and enumerating sequence reads. In some cases, the method further comprises aligning one or more of the sequence reads to a reference transcriptome or reference genome. In some cases, sequence reads of one or more pre-defined regions of the RNA are compared to a reference transcriptome or reference genome to determine regional expression counts.

In some cases, the regional expression counts of the one or more pre-defined regions are determined by hybridization. In some cases, the hybridization comprises contacting the RNA or cDNA with one or more probes. In some cases, the hybridization comprises analyzing the RNA or cDNA with a microarray. In some cases, the hybridization comprises determining the relative number of RNA or cDNA sequences that have annealed to one or more probes in one or more predefined region of a reference sequence to generate regional expression counts.

In some cases, the regional expression count of the one or more pre-defined regions is determined by amplification. In some cases, amplification comprises contacting the RNA or cDNA with one or more probes. In some cases, the amplification comprises analyzing the RNA or cDNA using qPCR or digital PCR. In some cases, results from the amplification-based quantitation within one or more pre-defined regions of the reference sequence are used to generate regional expression counts.

In some cases, the RNA comprises RNA obtained from cells that have been removed from the preimplantation embryo, or the cDNA comprises cDNA derived from RNA obtained from cells that have been removed from the preimplantation embryo. In some cases, the RNA comprises cell-free RNA. In some cases, the cell-free RNA is obtained from a liquid surrounding a preimplantation embryo, wherein the liquid comprises culture media. In some cases, the RNA comprises RNA obtained using a non-invasive method. In some cases, the RNA comprises RNA obtained using an invasive method. In some cases, RNA comprises RNA derived from the preimplantation embryo less than 1 hour, 6 hours, 12 hours, 1 day, 2 days, 3 days, 4 days, 5, days, 6 days, 7 days, 8 days, 9 days, 10 days, 2 weeks or 3 weeks after the initiation of RNA expression in the preimplantation embryo or after fertilization of the preimplantation embryo.

In another aspect, a method of determining a presence or absence of a genomic copy number alteration in an embryo is provided, the method comprising: a) obtaining a maternal sample comprising cell-free maternal and embryonic RNA; b) reverse transcribing the cell-free maternal and embryonic RNA to form cDNA; c) performing high-throughput sequencing of the cDNA to generate sequence reads; and d) analyzing the sequence reads to determine the presence or absence of the genomic copy number alteration in the embryo.

In another aspect, a method of determining a presence or absence of a genomic copy number alteration in an embryo is provided, the method comprising: a) obtaining a maternal sample comprising cell-free maternal and embryonic RNA; b) performing high-throughput sequencing of the RNA to generate sequence reads; and c) analyzing the sequence reads to determine the presence or absence of the genomic copy number alteration in the embryo.

In some cases, the maternal sample is a maternal blood sample.

The elements described above can be combined in any combination.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of a device of this disclosure are set forth with particularity in the appended claims. A better understanding of the features and advantages of this disclosure will be obtained by reference to the following detailed description that sets forth illustrative embodiments, in which the principles of a device of this disclosure are utilized, and the accompanying drawings of which:

FIG. 1 is a schematic flow diagram of clinical implementation of screening for genomic copy number abnormalities (CNAs) in embryos. The double line separates activities that can be done in the clinic (above the line) from those that can be performed the diagnostic laboratory (below the line). I. and II. Potential parents provide gametes or specimens that can be used to generate gametes. III. and IV. Embryos can be generated and cultured through the onset of expression of the embryonic genome. V. Samples containing RNA from embryo(s) can be obtained. VI. Samples can be processed to identify genomic copy number alterations. VII. The results of the copy number analysis can be interpreted clinically. VIII. Data can be stored and reports can be generated and transmitted to the clinical staff and patients. IX. The results of the RNA-based CNA detection can be incorporated with other clinical information for the embryos as well as the medical recommendations of clinical staff. X. A decision can be made by the parent(s) and medical staff for each embryo as to whether it is suitable for transfer. XI. These data can then be incorporated into final decisions for how embryos are to be handled.

FIG. 2 is a schematic diagram that demonstrates how a genomic copy number gain can affect the transcript levels for genomic loci. This figure depicts 2 embryos with different genotypes: a reference embryo that is disomic for a chromosome containing 3 loci and a sample embryo that is trisomic for this chromosome. Transcripts produced from these 3 loci are shown to the right with the number of copies indicating the amount of transcript produced by the locus. In comparing the relative amount of transcripts for each locus between the sample and reference, loci 1 and 3 show a 1.5 fold increase in the amount of transcript, which corresponds to the increase in the number of copies of these loci. In contrast, locus 2 shows a 0.25 fold decrease in expression. These relative alterations in expression can be used to identify this copy number abnormality. Loci 1 and 3 can be identified on the basis of looking for a positive correlation with copy number. Loci 2 can be identified provided that the negative response of this locus to the gain in copy number has been defined.

FIG. 3 is a schematic diagram that demonstrates how a copy number gain can influence allelic expression and allelic expression ratios. In this figure, the reference is depicted as being disomic for a chromosome, containing both paternal (P) and maternal (M) homologues whereas the sample is trisomic for this chromosome as a result of having 2 maternal homologues. The chromosome depicted has 3 loci that are transcribed with each harboring a single nucleotide polymorphism with the alleles indicated by white symbols and letters below. In the reference, all three polymorphisms are heterozygous while in the sample, the SNPs in loci 1 and 3 are heterozygous and the SNP in locus 2 is homozygous. When the expression of the parental haplotypes are compared between the sample and reference, loci 1 and 3 have a 2-fold increase of the maternal alleles whereas there is no increase for the paternal alleles. Locus 2 is not evaluated due to it being uninformative for allele analysis. When the expression of the alleles of loci are compared to each other by using an allele ratio such as the higher expressing to lower expressing, there is evidence of an imbalance of expression for loci 1 and 3 when compared to the reference. Locus 2 is not evaluated due to it being homozygous.

FIG. 4 is a schematic diagram of the effect of a loss of copy number on the heterozygosity of polymorphisms. In this figure, the reference has normal maternal (M) and paternal (P) homologues of a chromosome whereas the sample has a deletion of a segment of the maternal homologue encompassing loci 2 and 3. The deletion causes loci 2 and 3 to be monoallelic, a condition referred to as loss of heterozygosity.

FIG. 5 is a schematic diagram showing how a genomic copy number alteration can be detected by identification of a breakpoint. In this figure, the reference contains 2 normal copies of chromosomes, each harboring 4 loci. The sample below carries a chromosomal translocation that leads to a fusion locus (G/B) with duplication of loci C and D and deletion of H and part of G. Since the fusion locus is transcribed, the breakpoint can be identified by sequencing and finding either: (1) a ‘split read’ in which two segments of spanning reads map to different regions of the genome or (2) discordant read pairs in which the two end sequences of the clone align to regions of the genome that are not normally spaced or oriented as found in the clone.

FIG. 6 is a schematic diagram showing how a genomic copy number alteration can be detected by the presence of an expression signature. In the disomic sample, 2 chromosomes are shown (1-2), each containing 2 loci (A-D). Locus A positively regulates the expression of locus D (dashed lines). In an embryo with a trisomy for chromosome 1, the copy number gain has both a primary effect, increasing the expression of loci A and B due to a dosage increase (solid box), and a secondary effect, increasing the expression of locus D in response to the increase in the positive regulatory influence of locus A (double line box).

FIG. 7 is a schematic diagram presenting some approaches for generating preimplantation embryos.

FIG. 8 is a diagram showing images of preimplantation development of a human embryo and the biopsy procedures. The top panel of images shows the morphology of the embryo at roughly 24 hour interval from fertilization to the fifth day of development. Below the panel of embryo images are two exemplary images of biopsies being performed at days 3 and 5 of development. To the left of the embryo is a holding pipet that secures and positions the embryo and to the right is a smaller bore pipet that is used to obtain the specimen. For biopsies on day 5, a section of the mural trophectoderm (TE) can be obtained, which is located opposite to the inner cell mass (ICM).

FIG. 9 is a schematic diagram of types of nucleic acids that can be generated from RNA samples and the types of nucleic acids that can be analyzed. RNA is depicted in grey and DNA is depicted in black. The strand that is the same as the RNA is a solid line while the complementary strand is shown in dashed lines. Abbreviations include: amp—amplification, ivt—in vitro transcription, dp—dna polymerase, mda—multiple displacement amplification and spia—single primer isothermal amplification.

FIG. 10 is a schematic of several different methods that can be used to identify and quantitate nucleic acids. One method is to sequence the nucleic acids. The sequence can be used to determine identity and the number of reads can be used to quantitate the amount of nucleic acid present. Another method that can be used is to use probes of known sequence, hybridize the probes and nucleic acids and detect the annealed product (in dashed circle). The probe can define the identity and the amount that anneals can define the quantity. Another method is to amplify the sequence using one or more primers and a variety of amplification methods. The primer sequence(s) can determine the identity and the amount of amplification product can be used to determine the quantity. FIG. 10 discloses SEQ ID NO: 4.

FIG. 11 is a schematic diagram showing the steps that can be used to amplify cDNA from a sample. The steps can include the generation of a first strand through reverse transcription, the production of a second strand and then annealing. The first strand can be generated by including primers that bind to polyadenines at the 3′ terminus of some messenger RNAs and/or one or more primers that bind to other sequences to facilitate reverse transcription. The synthesis of the second strand can be done by approaches that include the addition of a polynucleotide sequence to the first strand (poly (dC) or poly (dA)) followed by the annealing of a primer to this sequence or the annealing of one or more primers to other sequences present or ligated to the first strand (NNN). The double stranded cDNAs can then be amplified through the use of sequences introduced into one or both primers (primers A and B).

FIG. 12 is a schematic diagram that depicts two methods for fragmenting amplified cDNAs for the purposes of generating a sequencing library. One method utilizes mechanical shearing and the other utilizes the Tn5 transpose tagmentation method. Once the cDNA has been fragmented and size selected, the library can be amplified using the adaptors present on the termini (arrowheads).

FIGS. 13A-13G depict exemplary steps involved in sequencing libraries using an Illumina/Solexa platform (Image adapted from Ansorge (2009) New Biotech 24: 195-203, incorporated herein by reference). A. Individual clones are affixed to a substrate. B. Free end of clone anneals to primer on substrate and begins bridge amplification. C. Bridging amplification results in the generation of replicates of the clone in the vicinity, known as a cluster. D. A sequencing primer is annealed. E. The first base is extended, read and deblocked. F. The process is repeated. G. Base calls are generated from the fluorescent signals.

FIG. 14 is a schematic flow diagram presenting the steps that can be involved in processing and analyzing raw data generated from sequencing-, hybridization- or amplification-based approaches for the purposes of detecting genomic copy number alterations.

FIG. 15 is a schematic diagram demonstrating how regional expression counts can be determined for various nucleic acid quantitation methods. In this example, a genomic region with the 2 chromosomal homologues is shown with 3 exons (black boxes). In this case, a region including exon 3 is deleted in one of the homologues. In this example, predetermined regions are defined by exons. For RNA-Seq, the expression count can be determined for each region by counting the number of reads that start within the exon. For hybridization-based methods, the intensity of the signals for the probe(s) that hybridize within the region can be summed or averaged. For amplification-based methods, the amplification-based quantitation data for amplicons located within regions can be used.

FIGS. 16A and 16B show an example of how expression signature-based detection of genomic copy number alterations can be performed. FIG. 16A is a Venn diagram presenting the results of a comparison of loci that are altered in expression in various trisomies, revealing 64 loci that are commonly dysregulated. These loci can be used to evaluate embryos for the risk of trisomy. In FIG. 16B, a hypothetical example shows the evaluation of several embryos for several of the observed alterations in locus expression. In this example, several loci from this panel are listed with the direction of alteration relative to euploid samples indicated. The relative expression of several embryos for these loci are evaluated and classified according to the relative change: <0.5(−−); 0.5-0.9 (−); >0.9-1.1 (=); >1.1-2 (+); >2 (++). In this hypothetical example, embryo 1 shows a high risk of a trisomy as the alterations are similar in direction for 6 of the 7 loci of the panel.

FIG. 17 is a schematic flow diagram demonstrating how various data and various copy number detection algorithms can be integrated. Raw data can be analyzed in toto to detect CNAs or a variety of algorithms can be run to detect CNAs for each type of data and then an algorithm can be used to integrate these results.

FIG. 18 is a schematic flow diagram showing how a genomic copy number alteration can be interpreted. In this approach, the copy number alteration can be compared to in house and reference databases to see if there are clinical data that may indicate whether or not the alteration is clinically benign. If not, the copy number alteration can be evaluated based on the understanding of the biology of the affected loci. Ultimately, CNAs can be classified as being clinically relevant, clinically benign or of unknown clinical significance.

FIG. 19 is an exemplary diagram of storage and dissemination of results from RNA analyses including CNA detection via computer.

FIG. 20 is a diagram showing the pairing of chromosomal homologues during meiosis I in a mouse carrying two Robertsonian chromosomes with a common arm (in white). When the chromosomes segregate by the alternate configuration (chromosomes I and IV segregate from chromosomes II and III), gametes with normal chromosomal complements can be formed. Whereas when the chromosomes segregate by the adjacent II configuration (chromosomes I and II segregate from chromosomes III and IV), gametes with a gain or loss of the monobrachial chromosome can arise. Adjacent II segregation occurs more frequently in the presence of these chromosomal abnormalities.

FIG. 21 is a representation of the workflow for generating, assessing the development of, genotyping, and isolating RNA samples from aneuploid mouse embryos.

FIG. 22 is a schematic diagram of the single primer amplification method used to amplify cDNA from mouse embryos. This figure was taken from the Nugen Ovation User Manual.

FIG. 23 is a Manhattan plot representing the fold changes in loci expression from mouse embryos with trisomy 10 as compared to normal disomic samples. The data are binned by chromosome number along the abscissa. Expression data for chromosome 10 are boxed.

FIG. 24 is a box plot graph showing the relative fold changes for the large input GM01201 sample compared to the reference. The expression data are divide into groups based on chromosomal location (designated chr). The box delineates the upper and lower quartiles and the horizontal bar represents the median.

FIG. 25 is a box plot graph showing the relative fold changes for the low input GM01201 sample compared to the reference. The expression data are divide into groups based on chromosomal location (designated chr). The box delineates the upper and lower quartiles and the horizontal bar represents the median.

FIG. 26 is a blox plot graph presenting relative expression data generated by comparing the simulated biopsy sample data from 2 embryos. The fold changes are presented on the ordinate. The relative expression data are grouped for each chromosome.

### DETAILED DESCRIPTION OF THE DISCLOSURE

### I. GENERAL TERMINOLOGY

The compositions and methods of this disclosure as described herein can employ, unless otherwise indicated, techniques of embryology, molecular biology (including recombinant techniques), cell biology, biochemistry, microarray and sequencing technology, which are within the skill of those who practice in the art. Such techniques include gamete isolation and handling, fertilization, embryo culture, embryo cryopreservation, embryo biopsy, RNA isolation, reverse transcription, nucleic acid amplification, massively parallel sequencing technologies, polymer array synthesis, hybridization of nucleic acid probes, detection of hybridization using a label and quantitative polymerase chain reaction methods. Specific illustrations of suitable techniques can be had by reference to the examples herein. Such techniques can be found in Fritz and Speroff, Eds Clinical Gynecologic Endocrinology and Infertility, (2010) Philadelphia: Lippincott Williams & Wilkins; Gardner et al Textbook of assisted reproductive techniques: laboratory and clinical perspectives, (2012) London: CRC Press; Green, et al., Eds., Genome Analysis: A Laboratory Manual Series (Vols. I-IV) (1999); Weiner, et al., Eds., Genetic Variation: A Laboratory Manual (2007); Dieffenbach, Dveksler, Eds., PCR Primer: A Laboratory Manual (2003); Bowtell and Sambrook, DNA Microarrays: A Molecular Cloning Manual (2003); Mount, Bioinformatics: Sequence and Genome Analysis (2004); Sambrook and Russell, Condensed Protocols from Molecular Cloning: A Laboratory Manual (2006); and Sambrook and Russell, Molecular Cloning: A Laboratory Manual (2002) (all from Cold Spring Harbor Laboratory Press); Stryer, L., Biochemistry (4th Ed.) W.H. Freeman, N.Y. (1995); Gait, “Oligonucleotide Synthesis: A Practical Approach” IRL Press, London (1984); Nelson and Cox, Lehninger, Principles of Biochemistry, 3^{rd} Ed., W.H. Freeman Pub., New York (2000); and Berg et al., Biochemistry, 5^{th} Ed., W.H. Freeman Pub., New York (2002) and Rodriguez-Ezpeleta, Bioinformatics for High Throughput Sequencing, Springer, New York (2012), Jin, Hailing Gassman and Walter, RNA Abundance Analysis, Humana Press, New York and Feuk, Genomic Structural Variants (2012) Springer, New York, all of which are herein incorporated by reference in their entirety for all purposes.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising”. The term “about” as used herein can refer to a range that is 15%, 10%, 8%, 6%, 4%, or 2% plus or minus from a stated numerical value.

### II. OVERVIEW

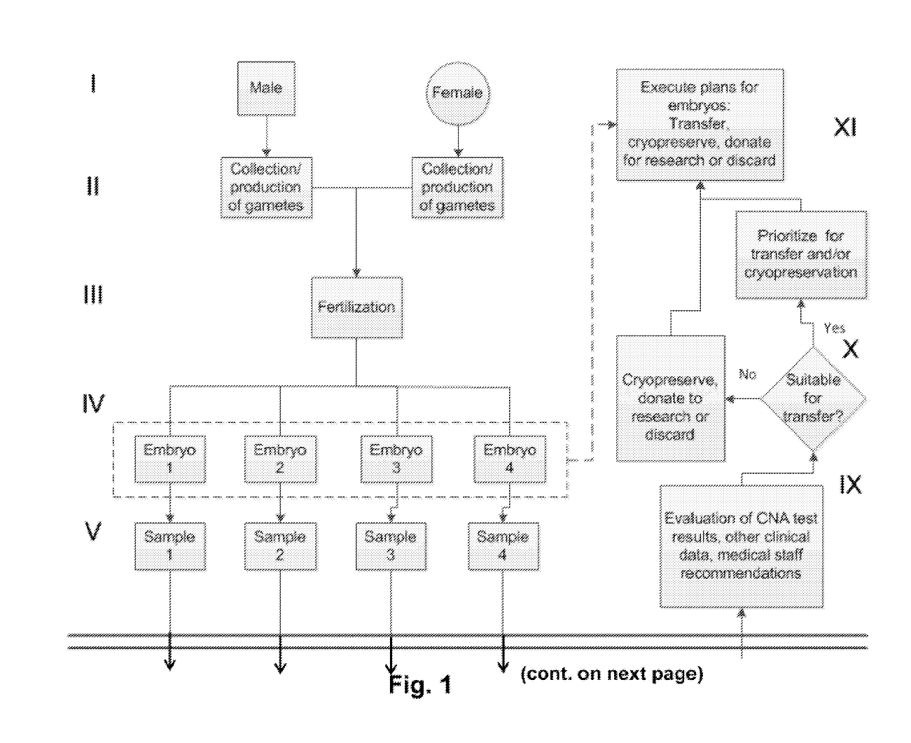

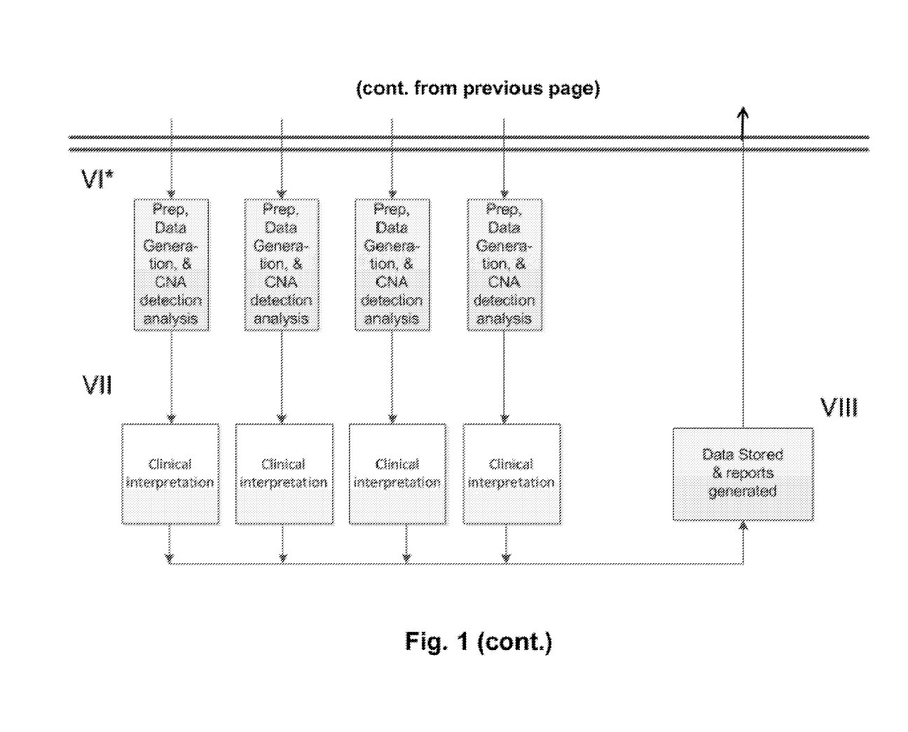

The present disclosure provides for compositions and methods for identifying genomic copy number alterations (CNA) through the analysis of RNA from an embryo, which can be referred to as RNA-based CNA detection (RCNAD). This approach can have clinical application in the evaluation of embryos before the establishment of pregnancy (see e.g., FIG. 1). In many cases, analysis of RNA can also be used to detect a variety of other genetic alterations and assess other biological characteristics of an embryo. CNAs encompass changes in the number of copies of genomic regions that involve one or more basepairs of the genome. CNAs can involve more than 10, more than 100, more than 1000, more than 10,000, more than 100,000, more than 1 million, more than 5 million, more than 10 million basepairs in the genome. CNAs include indels, copy number variants, insertions, deletions, segmental aneusomies, genomic disorders and aneuploidies.

The present disclosure provides for compositions and methods for identifying CNAs in embryos through analysis of RNA obtained from embryos or a derivative nucleic acid produced from the RNA. Three different approaches for RCNAD can be used independently or in combination to detect the presence of CNAs in embryos: regional expression-, breakpoint identification- and expression signature-based. The feasibility of a given approach for detecting a CNA can depend on the size and location of the CNA and the method(s) used for generating and analyzing the data.

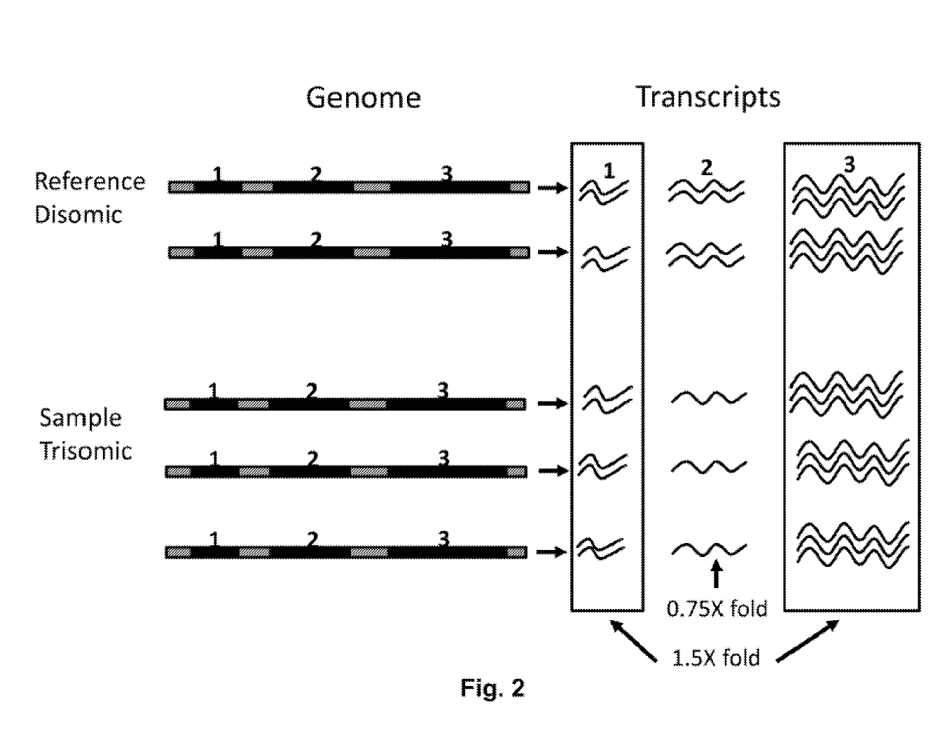

The regional expression-based approach can involve the identification of regions of the genome or corresponding transcriptome with altered expression relative to a reference. This regional expression-based approach is based on there being a sufficient proportion of transcribed loci within the CNA that are copy number sensitive (i.e., have a recognized and predictable response to a change in copy number). A locus can include any region of the genome that is transcribed. Dosage sensitive loci can make a region detectable by comparing the expression of loci from the affected region to those from a reference using one of a variety of algorithms and/or statistical ethods. For example, a trisomy can be detected due to altered expression of one or more dosage-sensitive loci located on the triplicated chromosome (see e.g., FIG. 2). Example 1 demonstrates that preimplantation mammalian embryos can have very high positive correlations between copy number and the level of expression of transcribed loci. This method can be used with expression data from loci and/or alleles (see e.g., FIGS. 2-4). This method of CNA detection can be used for evaluating the copy number of select region(s) of the genome or for surveying the entire genome. An example of evaluation of a select region of the genome would be for embryos produced by a parent who carries a balanced translocation. In some cases, a breakpoint associated with a CNA can be detected by the identification of a fusion locus in which the regions 5′ and 3′ to the breakpoint differ in their levels of expression. This discrepancy can be attributed to differences in the expression levels of the normal and fusion loci. In terms of genome-wide surveying, RCNAD can be used to screen embryos for aneuploidies (gains or losses of whole chromosomes) and subchromosomal alterations in copy number. This approach can have relevance for mammalian preimplantation embryos due to the high prevalence of CNAs that involve entire or large segments of chromosomes. The resolution of detection can be determined by the number of dosage-sensitive loci that are evaluated in the region(s) of interest and the methods of data generation and analysis.

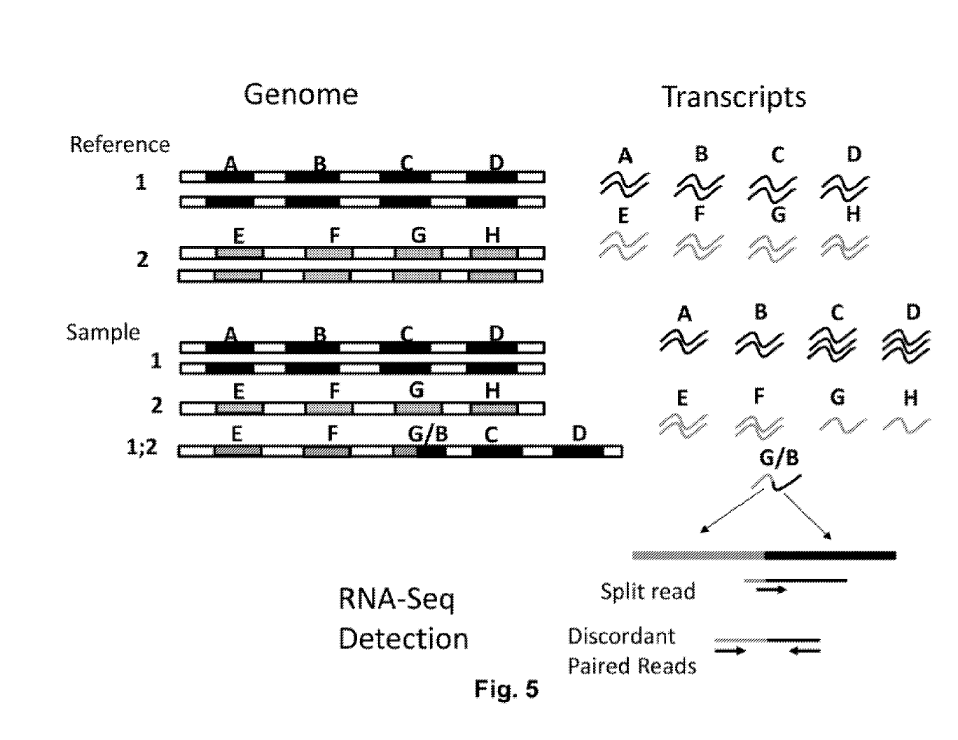

Another approach to detection of CNAs, which can be referred to as breakpoint identification-based CNA detection, identifies sequence alterations that can indicate the presence of a CNA (see e.g., FIG. 5). With the exception of aneuploidies, polyploidies and CNAs in repetitive sequences, other types of CNAs can be accompanied by novel sequence alterations. For example, deletions can have a breakpoint that joins normally distant sequences, insertions can have 2 novel breakpoints where the inserted DNA joins to sequences that are not normally juxtaposed and a translocation can fuse two sequences from different chromosomes (see e.g., FIG. 5). When breakpoints of structural genomic alterations reside within regions that are transcribed and incorporated into stable transcripts, these novel sequences can be detected using approaches such as RNA-Seq. When RNA-Seq is used, breakpoints can be detected by presence of ‘split reads’ in which some reads can include the breakpoint (i.e., the read contains sequences that align to regions of the genome that are not contiguous and cannot be explained by normal or trans-splicing of the transcript) or sequencing of the ends of the library clone (paired end sequencing) and showing that the two sequences align to regions of the genome that are not consistent with estimated size of the intervening sequence in the library and cannot be explained by normal or trans-splicing.

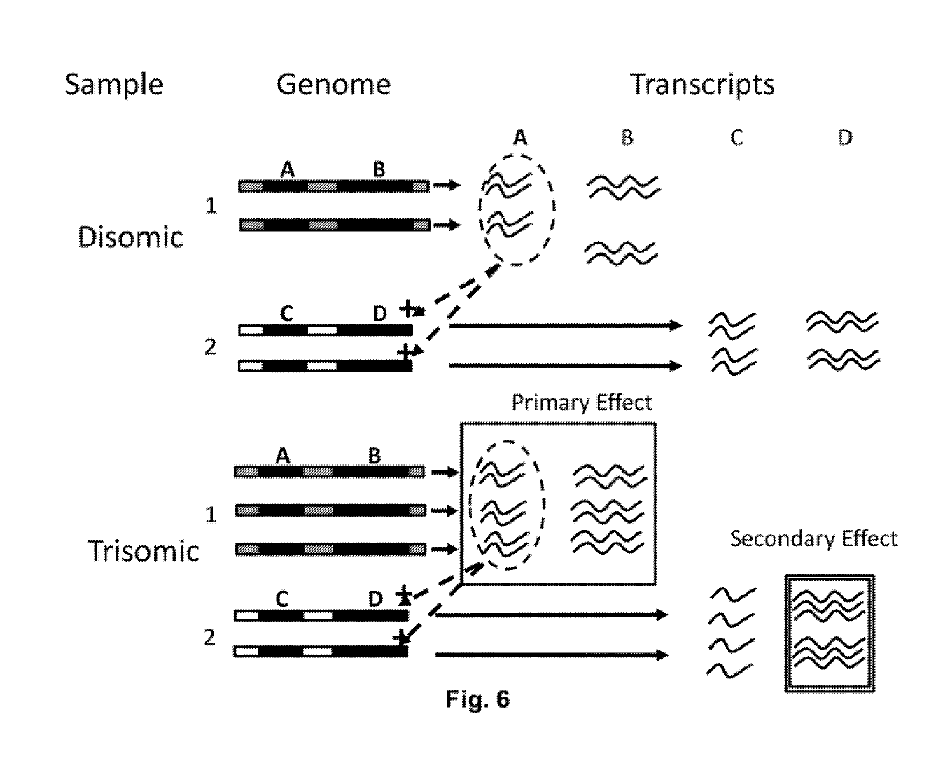

A third approach that can be used to identify embryos that carry CNAs can rely on the detection of alterations in the transcriptome that signal the presence of one or more CNAs, a method that can be referred to as expression signature-based CNA detection (ESCNAD) (see e.g., FIG. 6). For this approach, expression profiles of embryos with CNAs can be evaluated to identify profiles that can serve as markers of CNAs. These profiles can include all alterations in the transcriptome rather than just the primary ones (i.e., ones that are in response to the dosage alteration) used for the regional expression-based approach. Some profiles can be more specific, indicating the presence of one or a small number of CNAs whereas others can be more general, signaling the presence of a larger class of CNAs.

These three approaches to CNA detection can be used independently or in any combination. Since these methods provide complementary information, the combined use of these methods can improve the ability to detect CNAs accurately. Screening embryos for CNAs using any of the above methods can involve one or more steps. In some cases, the first step can be generating or retrieving embryos. A sample containing RNA produced by the embryo can be obtained. A number of optional processing steps can be performed on the sample to generate a sufficient quantity of the appropriate form of nucleic acid for analysis. For the regional expression-based method of detection, any one of a number of analytic methods can then be performed to determine the expression levels of one or more RNAs in a region of the transcriptome or genome of the sample. The methods can include sequencing-, hybridization- and amplification-based approaches. Following generation of the raw data from these methods, the data can then be analyzed by one or more algorithms executed by one or more computer processors to identify CNAs.

For breakpoint identification-based CNA detection, sequence data of transcripts can be evaluated. RNA-Seq can be used for generating sequence data. The sequence data derived from the RNA can be evaluated by a number of algorithms that can detect breakpoints within sequence reads.

An expression signature-based CNA detection can involve evaluating the RNA profile from an embryo to determine if it has a profile that has been recognized to be associated with a CNA. Methods that broadly survey the transcriptome, such as sequencing- and hybridization-based methods, can be well suited for this method of detection. A variety of algorithms can be used to identify common expression profiles for various groups of CNAs, e.g., once a large number of embryos with CNAs have been evaluated. Expression data from embryos can be evaluated to determine whether the CNA profile(s) are present, e.g., once a profile for one or more CNAs is identified.

The results of these analyses for CNAs can be used to generate a report that can be provided to appropriate parties for clinical and/or research purposes. The results of this testing can impact clinical decisions pertaining to the embryo (see e.g., FIG. 1). Some of the identified CNAs and other additional information obtained from these analyses can impact the health of the embryo, its subsequent development, or health at later stages of development. In some cases, compositions and methods of this disclosure can provide information useful in making decisions regarding whether an embryo or ensuing fetus or offspring should undergo additional testing. In some instances, the compositions and methods of this disclosure can provide information that can be used to determine the fate of the embryo, which can include transfer to the female genital tract, cryopreservation, donation to research, donation to another female or couple for the purposes of establishing a pregnancy, disposal or additional culture followed by one of the previously mentioned fates. In some cases, the embryo can be cryopreserved before the results of the CNA analysis are available. In this situation, the results can impact the decision on whether to thaw or warm an embryo for any of the previously mentioned fates or to maintain the embryo in cryopreservation.

In concert with CNA detection, the data produced for this analysis can also be used to determine if other genetic alterations or traits are present or have been inherited as well as to assess the health and developmental competence of the embryo. A genetic alteration can be any change in genomic sequence relative to another sequence, e.g., a reference sequence. Examples of genetic alterations include mutations, which can be considered to cause disease, and polymorphisms, which are alterations present in greater than 1% of the population. Genetic alterations include, but are not limited to, point mutations, transversions, transitions, nonsense mutations, frame shift mutations, repeat mutations, translocations, inversions and duplications, small nucleotide polymorphisms (SNPs), simple sequence repeats and copy number abnormalities (CNAs). Genetic alterations can cause genetic disease, contribute to susceptibility of disease or contribute to one or more traits. A genetic alteration or abnormality can occur in the coding or non-coding regions of the genome. In some cases, genetic alterations can be located in regions of the genome that are transcribed and represented in stable RNAs. These alterations can be detected directly through analyses of RNA. In other cases, genetic alterations are not in regions that are transcribed or produce sufficient amounts of RNA so that they cannot be detected directly. In some of these cases, the alteration can be detected indirectly through the identification of primary or secondary alterations in RNA. In some cases, the alteration can exert a primary effect on one or more RNAs by altering production, processing or stability of the transcript(s). In other cases, the alteration can affect a locus that in turn can affect the production, processing or stability of RNA from another locus. In some cases, these secondary changes can be used to infer the presence of a genetic alteration. In other cases, the inheritance of a genetic alteration can be detected indirectly through linkage analysis by assessing the inheritance of linked sequence variants that can be detected in the RNA. The detection of genetic alterations can be used to determine the cause of a disease, identify the susceptibility to a disease or determine the presence or absence of a trait.

Analysis of RNA can provide additional information pertaining to the biology of the embryo. In some cases, analysis of RNA can identify epigenetic abnormalities through alterations in the expression of loci that are regulated by an epigenetic mechanism such as genomic imprinting. In other cases, analysis of RNA can provide insight into the developmental stage, health or developmental potential by evaluating patterns of expression of one or more transcribed loci. RCNAD can also be combined with one or more evaluations of the embryo that are not RNA-based. Additional analyses can include DNA-based analyses of the nuclear or mitochondrial genomes, assessment of metabolism, evaluation of proteins produced by the embryo or assessment of morphology of the embryo.

### III. EMBRYO GENERATION

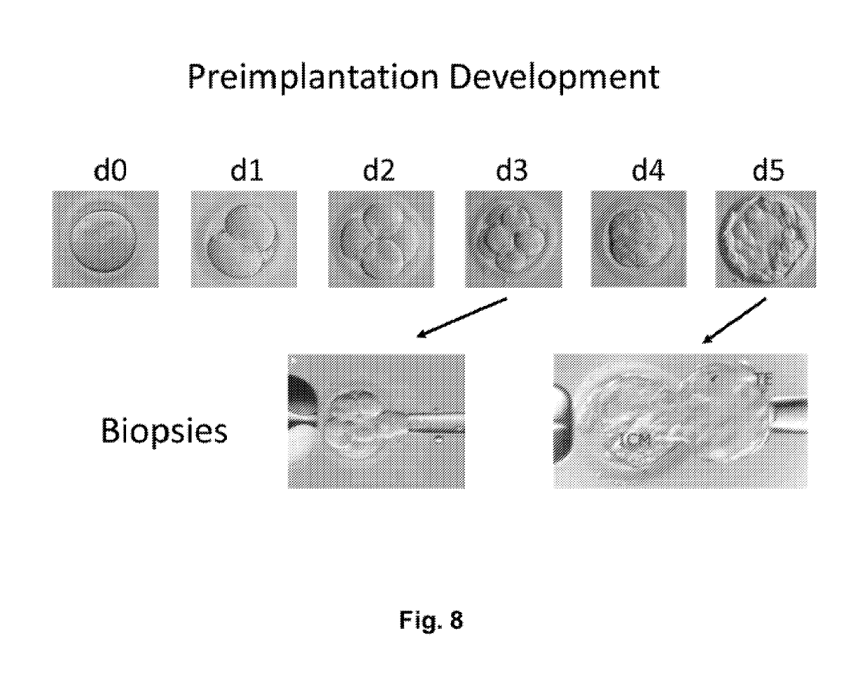

The source of samples for the compositions and methods of this disclosure can be produced by one or more embryos from any species. One or more embryos can be at any developmental stage after RNA is expressed by its genome. An embryo can be from a vertebrate or an invertebrate. In some cases, an embryo is from a mammal. A mammalian embryo can be from a human, a non-human primate (e.g., chimpanzee, orangutan, or gorilla), livestock, cow, horse, pig, sheep, goat, cat, dog, buffalo, guinea pig, hamster, rabbit, mice, domesticated species or endangered species. In some cases, diagnostic approaches can be applied within minutes, hours, days, or weeks following the initiation of expression of the embryonic genome or within minutes, hours, days, or weeks of fertilization. The methods herein can be applied to a zygote, cleavage-stage embryo, morula, blastocyst, early blastocyst, expanding blastocyst, expanded blastocyst, hatching blastocyst, hatched blastocyst or an embryo of about 1, 5, 10, 15, 20, 50, 100, 150 or 200 cells or at least 1, 5, 10, 15, 20, 50, 100, 150, or 200 cells, or less than 500, 400, 300, 200, 100, 50, 40, 30, 20 or 10 cells, or an embryo with about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149,150, 151, 152, 153, 154, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199 or 200 cells (see e.g., FIG. 8).

In some cases, the methods herein can be applied to a mammalian embryo after expression of the embryonic genome and up until the embryo is transferred to the female genital tract to allow for normal subsequent development. In some cases, this period extends to the period when the embryo naturally implants into the uterine wall. In some instances, such period is extended, e.g., by allowing the embryo to be maintained in culture for a longer period than the natural preimplantation period, or by cryopreservation.

In some cases, sample processing and analysis can be performed immediately following the biopsy so that the results can be generated and conveyed to the medical staff and patient(s) in a timing that permits the results to be incorporated into the decision of whether or not to transfer the embryo and, if deemed appropriate, to transfer the embryo to the female reproductive tract without the embryo being cryopreserved. In some cases, the embryo is cryopreserved following acquisition of the sample and the sample can be processed and analyzed either immediately or at a later date.

In some cases, the compositions and methods of this disclosure comprise the generation of one or more embryos by any means capable of producing a healthy, normal liveborn offspring, including intercourse or mating.

### III.A. Oocyte Generation

Gametes can be retrieved from the female or produced by a method that generates one or more female gametes capable of supporting the production of a healthy liveborn. Gametes or cells/tissue capable of generating gametes can be isolated from vertebrate or invertebrate animals. The animal can be a mammal, including a human, non-human primate (e.g., chimpanzee, orangutan, or gorilla), cow, horse, pig, sheep, goat, cat, dog, buffalo, guinea pig, hamster, rabbit, mice, domesticated species, or endangered species. Suitable gametes for use in the disclosure can include but are not limited to immature oocytes and mature oocytes. In some cases, the oocytes can be collected from normally cycling females while in other instances the oocytes can be collected after administration of one or more fertility agents or fertility enhancing agents (e g , inhibin, inhibin and activin, clomiphene citrate, human menopausal gonadotropins including follicle-stimulating hormone (FSH), or a mixture of FSH and luteinizing hormone (LH), and/or human chorionic gonadotropins) to the oocyte donor or an obtained specimen. In some embodiments of the disclosure, the oocytes are aged (e.g., the oocytes are derived from a woman 35 years or older, 40 years or older, or from animals past their reproductive prime).

In some cases, oocytes can be obtained through a controlled ovarian stimulation protocol to promote ovarian follicle growth and maturation. For example, in humans, hormonal treatment cycles can begin on the third day of menstruation, constituting about ten days of daily subcutaneous injections of protein hormones, termed gonadotropins. These injections can be delivered under close monitoring by a health-care provider. The monitoring can involve evaluating estradiol hormone levels and/or ovarian follicular growth. The prevention of spontaneous ovulation can involve utilization of other hormones such as gonadotropin-releasing hormone (GnRH) antagonists or GnRH agonists that can block a natural surge of luteinizing hormone (LH). A protocol for controlled ovarian stimulation can be individualized for patients based on response to hormones and/or past medical history. In some cases, oocytes can be retrieved using minimal stimulation or during natural cycles (i.e., no exogenous hormonal stimulation). When follicles are of a proper stage of development for retrieval, e.g., just prior to ovulation, the oocytes can be retrieved using a method such as transvaginal, ultrasound-guided follicular aspiration. In other cases, the follicles can be aspirated by perurethral/transvesical ultrasonographic puncture or retrieved laparoscopically. Once the follicular fluid is removed from the follicle, the oocytes can be located within the fluid using microscopy, inspected, and suitable specimens can be placed into culture medium in an incubator. Oocytes can also be cryopreserved, e.g., if the fertilization is to be performed at a later date.

Another example method of generating oocytes as provided by the compositions and methods of this disclosure can be to obtain immature follicles or oocytes and mature them in vitro under conditions such as those used in the art to promote oocyte maturation (e.g., see U.S. Pat. Nos. 5,882,928 and 6,281,013, incorporated by reference herein).

Another example method of obtaining oocytes can comprise isolating oocytes that have developed from ovarian stem cells isolated from one or more ovaries (e.g., see White, et al. (2012) Nature Medicine 18: 413-422, incorporated by reference herein).

Another method of obtaining oocytes can be through the acquisition of ovarian tissue followed by culture in vitro or transplantation, autologous or heterologous. In some cases, the ovarian tissue can be cryopreserved prior to culture or transplantation.

### III.B. Sperm Generation

Male gametes (i.e., sperm) can be obtained for embryo generation. Male gametes can be retrieved by ejaculation as a result of intercourse, masturbation, electrical or vibratory stimulation to the prostate or penis, puncture of the spermatic ducts, or testicle biopsy. In some cases, sperm can be collected from urine. In some cases, e.g., in severe cases of low or no sperm count, sperm or spermatids can be retrieved through the microsurgical procedures that include microsurgical sperm aspiration from the epididymis (MESA), percutaneous sperm aspiration from the epididymis (PESA), biopsy and sperm extraction from the testicle (TESE), or percutaneous sperm aspiration from the testicle (TESA). Male gametes can also be produced in vitro from the culture of testicular tissue or stem cells.

### III.C. Embryo Generation

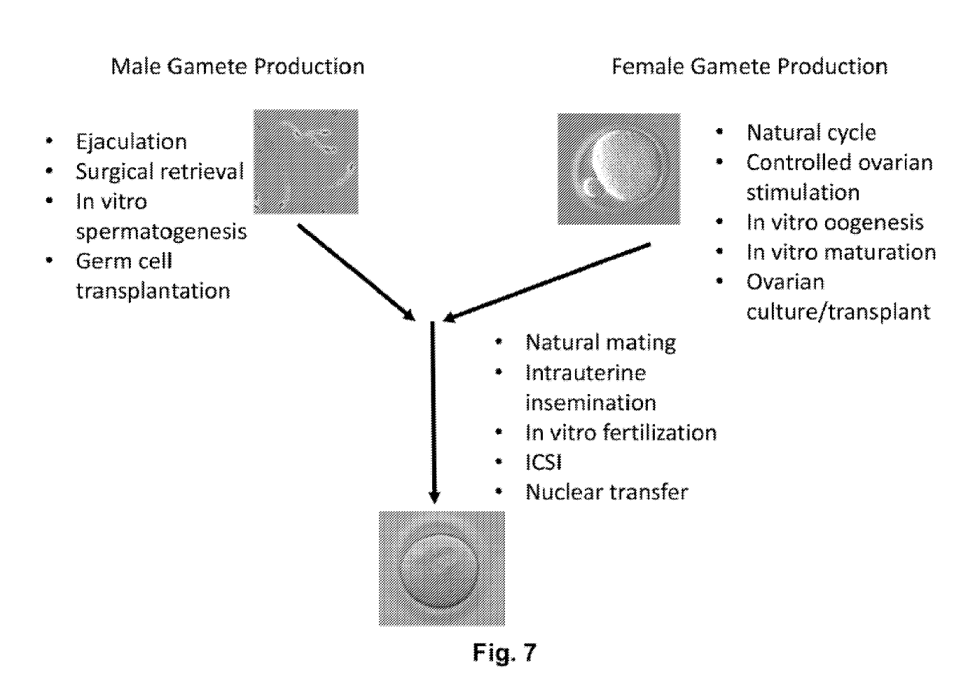

A variety of approaches can be used to generate embryos (see e.g., FIG. 7). In some cases, embryos can be generated through in vitro fertilization. In other cases, embryos can be produced through fertilization in vivo. In some cases, embryos can be produced by intercourse. In some cases, fertilization can be facilitated by intracytoplasmic sperm injection, which can comprise injecting a single sperm or spermatid into an egg. In some cases, embryos can be produced by co-incubating multiple sperm or spermatids and one or more eggs for a defined time period in conditions that facilitate fertilization, often referred to as in vitro fertilization (IVF, e.g., see U.S. Pat. Nos. 6,610,543 and 6,130,086, incorporated by reference herein).

In some cases, embryo production can comprise nuclear transfer from a donor cell into an enucleated oocyte or zygote. A diploid nucleus or two haploid nuclei can be transferred from the donor cell(s). Fertilization can be assessed by detecting the presence of pronuclei within hours after fertilization and/or mitotic division within 24 hours following fertilization.

### III.D. Embryo Culture, Storage and Cryopreservation

After fertilization, embryos can be maintained in conditions that can promote further development using known methods. For example, embryos can be maintained in small drops of culture medium on culture dishes that are overlaid with mineral or paraffin oil. These dishes can be maintained in an incubator, and the incubator can provide an environment optimized for embryonic health and development. Typical conditions can include a temperature approximating that found in vivo (e.g., about 35 to about 37° C.), a sub-ambient concentration of oxygen (e.g., 5%) and/or elevated concentration of CO₂ (e.g., about 5 to about 6%). The developmental progression and potentially other physiologic parameters of the embryo can be followed serially throughout the culture period (see e.g., FIG. 8). Mammalian embryos can be maintained in culture for a period up to the length of the natural preimplantation period. For example, human embryos can be maintained in culture for about, up to, more than, or at least 1, 2, 3, 4, 5, 6, 7, 8, 9, or 10 days. A number of other culture environments can be used in which a number of components or features of the system differ, including the volume of culture media, shape of the culture vessel, composition of vessel substrate, composition of culture medium, use of static or dynamic culture systems, mechanical or flow-induced movement of embryos, circulation or exchange of media, type of incubator and physiologic monitoring and imaging systems. Embryos can be cryopreserved at any time point during this period using techniques that are known in the art. Embryos can be cryopreserved by vitrification or slow programmable freezing. Cryopreservation techniques can comprise addition of one or more cryoprotectants to an embryo sample prior to cooling. Cryoprotectants used for cryopreservation include, but are not limited to, dimethyl sulphoxide, ethylene glycol, propylene glycol, 1,2-propanediol, 2,3-butanediol, methanol, dimethylacetamide, sucrose, trehalose and glycerol. A variety of devices have been developed to facilitate vitrification and storage of embryos (for review, see Arav (2014) Theriogenology 81: 96-102, incorporated by reference herein). Embryos can be cryopreserved at the 2, 4, 8-cell, compacting, morula or blastocyst stage. Blastocysts can be collapsed before cryopreservation. In some species, embryos can be induced to go into diapause, a state of arrested development, in vitro or in vivo to allow for temporary storage of embryos.

### IV. ACQUISITION OF RNA SAMPLES FROM EMBRYOS

A sample containing RNA can be obtained from the embryo. Such sample can be obtained at any appropriate time during the preimplantation or at any other time as described above. For example, a sample can be obtained from an embryo of about 1, 5, 10, 15, 20, 50, 100, 150 or 200 cells or at least 1, 5, 10, 15, 20, 50, 100, 150, or 200 cells, or less than 500, 400, 300, 200, 100, 50, 40, 30, 20 or 10 cells. The sample can include one or more forms of RNA or all forms of RNA expressed from cells of the embryo. RNAs obtained from an embryo can include any one or more of the following types RNA: messenger RNA (mRNA), ribosomal RNA (rRNA), transfer RNA (tRNA), nuclear RNA (nRNA), non-coding RNA (ncRNA), small interfering RNA (siRNA), small hairpin RNA (shRNA), small nuclear RNA (snRNA), small nucleolar RNA (snoRNA), small cajal body RNA (scaRNA), microRNA (miRNA), piRNA (Piwi-interacting RNA), double stranded RNA (dsRNA), ribozyme and riboswitch. In some cases, the RNA is messenger RNA. The amount of RNA obtained in the sample can be more than 1, 5, 10, 20, 30, 40, 50, 60, 70, 80, 90, 100, 110, 120, 130, 140, 150, 160, 170, 180, 190, 200, 210, 220, 230, 240,250, 300, 400 picograms of total RNA. The amount of polyadenylated RNA obtained can be more than 1, 5, 10, 50, 100, 200, 300, 400, 500, 600, 700, 800, 900, 1000, 1500, 2000, 2500, 3000, 3500, 4000, 4500 or 5000 femtograms.

The sample can be obtained using an invasive method or non-invasive method. An invasive method can involve removal of cellular or subcellular material from the embryo. A noninvasive method can involve collecting cells, subcellular material or RNA that are naturally released from the embryo.

### IV.A. Invasive methods for obtaining an RNA sample

The methods and compositions of this disclosure provide for any invasive method that can yield a sample containing RNA that is suitable for analysis. In some cases, a sample can be obtained by biopsying the embryo to remove one or more cells from the embryo using techniques known in the art (see e.g., Xu and Montag (2012) Seminars in Reproductive Medicine 30: 259-266, incorporated by reference herein). Preimplantation embryos can be biopsied at any stage beyond the 2-cell stage or the timepoint at which the embryonic genome is being expressed (see e.g., FIG. 8). In some compositions and methods of this disclosure, the embryo can be biopsied at the blastocyst stage (see e.g., FIG. 8). Biopsy at this stage can involve the removal of trophectodermal cells that enclose the fluid-filled blastocoel and inner cell mass. In some cases, cells from the mural trophectoderm can be removed. In the case of humans, for example, a blastocyst can be biopsied on day 5 or day 6 following fertilization (i.e., 120-144 hrs post fertilization) using standard methods, such as those described in McArthur, et al. ((2008) Prenatal Diagnosis 28: 434-442, incorporated by reference herein). Generally, the trophectoderm can be promoted to herniate out of the zona pellucida (ZP) through a previously introduced breach. In some cases, the breach can be introduced by a diode near-infrared laser such as the Octax or Fertilase (MTM), Saturn 5 (RI) or Zilos-tk (Hamilton Thorne) lasers. In other embodiments, this breach can be created through the use of a mechanical means (e.g., blade or needle), a chemical or enzymatic means (e.g., acidic Tyrode's solution) or a thermal means (e.g., direct contact with a heating element). In the case of human embryos, the ZP breach can be performed on day 3 of 4 of culture. Blastocysts with herniation of the trophectoderm through the trophectoderm can be used for biopsy. Blastocysts that have fully hatched from the zona pellucida and those that have not hatched at all can also be biopsied. In the case of fully enclosed blastocysts, the breach previously introduced into the zona pellucida can be used, or the breach can be enlarged, or a new breach can be made to obtain a sample. In other cases, the ZP is not breached until immediately prior to biopsy.

In the some cases, fresh blastocysts (embryos that have not been cryopreserved) can be biopsied. In other cases, biopsies can be performed on embryos generated from cryopreserved gametes or from embryos that have been previously cryopreserved. The period of cryopreservation can be days, weeks, months, years, or decades.

During biopsy, blastocysts can be placed in individual small drops of culture medium with oil overlays and can be transferred to an inverted microscope with a heated stage. The embryo can be secured by gentle suction to a thick-walled, blunt-ended pipet, known in the art as a holding pipet. The holding pipette can be maneuvered using a micromanipulator. The embryo can be oriented so that the section of the trophectoderm that is to be biopsied is oriented toward a smaller bore biopsy pipet. If the section to be biopsied is still contained within the ZP, a hole can be introduced into the ZP adjoining the area to be biopsied. A biopsy can be obtained by first either attaching the biopsy pipet to the area to be biopsied or drawing a small portion of the trophectoderm into the pipet's lumen with the aid of micromanipulation equipment to orient and move the specimen and a microinjector or other equipment that enables gentle negative and positive pressure to the applied to the pipet. A near-infrared laser can be used to detach a small segment of the trophectoderm containing more than 1-20 cells using multiple low power laser pulses. In some cases, more than one biopsy can be performed.

Other methods can be used to secure and manipulate the embryo. For example, methods can include an application that uses suction or physical constraint to keep the embryo at a defined location. In some cases, optical tweezers can be used to hold the embryo.

Other methods can be used to release the biopsy sample from the embryo. In some cases, a biopsy sample can be physically dissociated from the embryo using only the holding and biopsy pipets, e.g., dragging the biopsy pipet across the face of the holding pipet. In other cases, the biopsy can be cut from the embryo, e.g., using a blade or other cutting device.

Further, chemical and/or enzymatic methods can be used to release the biopsy sample from the embryo. In some cases intercellular connections or bridging cells can be disrupted by localized delivery of these disrupting agents. Chemical agents can include but are not limited to detergents or hypotonic solutions. Enzymatic agents include, but are not limited to, trypsin and proteinase K. The methods and compositions of this disclosure provide for any suitable method or combination of methods that can obtain one or more biopsy specimens.

In some cases as provided by this disclosure, the embryo can be biopsied at an earlier or later stage during development than the blastocyst stage. For earlier stages, any stage can be analyzed that follows activation of the embryonic genome, which can correspond to between about 24 to about 48 hours after fertilization in human embryos. In some cases, the earlier stage can be at the early cleavage stage in which there are 6-10 cells (see e.g., FIG. 8). At this stage, which can correspond to the 3^{rd} day following fertilization, the embryo can be transferred to media lacking divalent cations and/or containing chelating agents to promote dissociation of the blastomeres. Using micromanipulator and laser equipment as described herein, the ZP can be breached and 1 or 2 blastomeres can be removed using a biopsy pipet. In other cases, embryos can be split at the 2-8 cell stage (see Tang (12) Taiwanese J of Obstet Gyn S1: 236-9, incorporated by reference herein). In this case, one embryo can be sampled or used in its entirety for genetic analyses while the other can be reserved to establish a pregnancy if appropriate. In some cases, a system that is capable of simultaneously biopsying multiple embryos can be used.

In some cases, a biopsy can include at least 1, 2, 3, 4, 5, 6, 7, 8, 9, 10, 11, 12, 13, 14, 15, 16, 17, 18, 19, 20, 50, or 100 cells. In some cases, cells obtained for biopsy can comprise at most 500, 400, 300, 200, 100, 50, 40, 30, 20, or 10 cells. In some cases, cells obtained for biopsy comprise about 1, 2, 3, 4, 5, 6, 7, 8, 9, 10, 11, 12, 13, 14, 15, 16, 17, 18, 19, 20, 50, or 100 cells.

In some cases, the biopsy can be performed to remove one or more subcellular compartments of a cell rather than an intact cell. Subcellular compartments can include the nucleus, mitochondria and cytoplasm. Subcellular sampling can be performed using very fine gauge biopsy pipets with or without the aid of piezo.

In some cases, cells can be lysed in situ and the lysate containing RNA can be obtained immediately following lysis. In this method, a lysis method as described below can be delivered locally to lyse one or more embryonic cells. The lysed cellular content can then be immediately retrieved through aspiration.

In some cases, cells can be lysed in situ and the lysate containing RNA can be obtained during the biopsy process.

In some cases, lysates or subcellular components can be obtained from at least 1, 2, 3, 4, 5, 6, 7, 8, 9, 10, 11, 12, 13, 14, 15, 16, 17, 18, 19, 20, 50, or 100 cells. In some cases, lysates or subcellular components can be obtained from at least about 1, 2, 3, 4, 5, 6, 7, 8, 9, 10, 11, 12, 13, 14, 15, 16, 17, 18, 19, 20, 50, or 100 cells. In some cases, lysates or subcellular components can be obtained from about 1, 2, 3, 4, 5, 6, 7, 8, 9, 10, 11, 12, 13, 14, 15, 16, 17, 18, 19, 20, 50, or 100 cells.

In some cases, a sample containing RNAs produced by the embryo can be obtained from blastocyst stage embryos by obtaining fluid from the blastocoel cavity.

### IV.B. Noninvasive acquisition of RNA samples

In some cases, samples can be obtained without the removal of cells, subcellular material or fluid from the embryo (i.e., not affecting the integrity of the developing embryo). Embryonic cells can be obtained without a biopsy procedure through the collection of cells that have been released from the embryo. These cells can be collected from the culture medium or by collecting cells that are contained within or adherent to the zona pellucida (ZP) following removal and/or collection of the ZP.

A sample of cell-free RNA released from an embryo can also be obtained noninvasively for the compositions and methods of this disclosure. In some cases, cell-free RNA can be obtained from the embryo culture medium. In other cases, RNA that is contained within or adherent to the ZP can be isolated following removal and/or collection of the ZP. In other cases, RNA can be obtained from both culture medium and the ZP. In some cases, RNAse inhibitors and RNA stabilizing agents can be added to the medium to maintain integrity of the RNA before and during collection. RNAse inhibitors can include proteins, antibodies and chemicals that can inhibit the activity of one or more ribonucleases that may be present in the culture medium or introduced during sample collection and processing. RNAse inhibitor proteins include the mammalian ribonuclease inhibitor protein, which can be isolated in its natural form or produced as a recombinant protein with or without modifications. Antibodies that inhibit RNAse activity have been identified and are commercially available. Chemicals that inhibit RNAse activity include nucleosides, detergents and oxidizing agents. RNA stabilizing agents include commercial products such as RNALater (Qiagen), RNA Stabilizer (Wako) and DNA/RNA Shield (Zymo Research).

In other cases, cell-free RNA samples can be obtained through the isolation of extracellular vesicles including microvesicles and exosomes that can be released from the embryo. These extracellular vesicles can be isolated from the culture medium that bathes embryos through a variety of techniques including differential centrifugation, sucrose gradient centrifugation, microfiltration, antibody-mediated isolation techniques that employ magnetic beads or microfluidic devices to facilitate antibody-ligand binding, washing and vesicle isolation (see Momem-Heravi (12) Biol Chem 10: 1253-62, incorporated by reference herein).

In other cases, embryonic cell-free RNA can be isolated from bodily fluids of a mother including but not limited to blood, serum, plasma, genital tract secretions or washings, vitreous, sputum, urine, tears, perspiration, saliva, mucosal excretions, mucus, spinal fluid, lymph fluid and the like.

Isolation and extraction of cell-free RNA can be performed through a variety of techniques. In some cases, collection can comprise aspiration of a fluid from a subject using a syringe. In other cases collection can comprise pipetting or direct collection of fluid, i.e. culture media, from a vessel or droplet.

In some cases, the sample for RNA analysis can be obtained immediately following collection of the culture medium or the noninvasive sample. In other cases, the noninvasive sample can be stored, and then the sample for RNA analysis can be taken from this sample at a later date. In some cases, the noninvasive sample can be stored frozen. In other cases, the sample can be stored unfrozen. In some cases, RNAse inhibitors or stabilizing agents can be added to maintain integrity of the RNA as described above. In cases in which cells or extracellular vesicles are collected, agents can be added to stabilize the cells or vesicles.

### IV.C. Timing of Sample Acquisition

In some cases, invasive or noninvasive samples can be obtained at least 1 min, 10 min, 30 min, 1 hour, 2 hours, 5 hours, 12 hours, 1 day, 2 days, 3 days, 4 days, 5 days, 6 days, 7 days, 8 days, 9 days, 10 days, 1 week, 2 weeks or, 3 weeks after fertilization of the embryo (not including cryopreservation or sample storage time). In some cases, cells obtained for biopsy of an embryo can be obtained at most 10 weeks, 8, weeks, 6 weeks, 4 weeks, 3 weeks, 2 weeks, 1 week, 6 days, 5, days, 4 days, 3 days, 2 days or 1 day after fertilization of the embryo (not including cryopreservation time or sample storage time).

In some cases, the invasive or noninvasive sample can be obtained at least 1 min, 10 min, 30 min, 1 hour, 2 hours, 5 hours, 12 hours, 1 day, 2 days, 3 days, 4 days, 5 days, 6 days, 7 days, 8 days, 9 days, 10 days, 1 week, 2 weeks or, 3 weeks after initiation of expression of the embryonic genome (not including cryopreservation time or sample storage time). In some cases, the invasive or noninvasive sample can be obtained at a time of no more than 1 min, 10 min, 30 min, 1 hour, 2 hours, 5 hours, 12 hours, 1 day, 2 days, 3 days, 4 days, 5 days, 6 days, 7 days, 8 days, 9 days, 10 days, 1 week, 2 weeks or 3 weeks after initiation of expression of the embryonic genome (not including cryopreservation time or sample storage time). In some cases, invasive or noninvasive samples can be obtained about 1 min, 10 min, 30 min, 1 hour, 2 hours, 5 hours, 12 hours, 1 day, 2 days, 3 days, 4 days, 5 days, 6 days, 7 days, 8 days, 9 days, 10 days, 1 week, 2 weeks, or 3 weeks after initiation of expression of the embryonic genome (not including cryopreservation time or sample storage time).

### V. SAMPLE PREPARATION AND GENERATION OF RAW TRANSCRIPTOME DATA

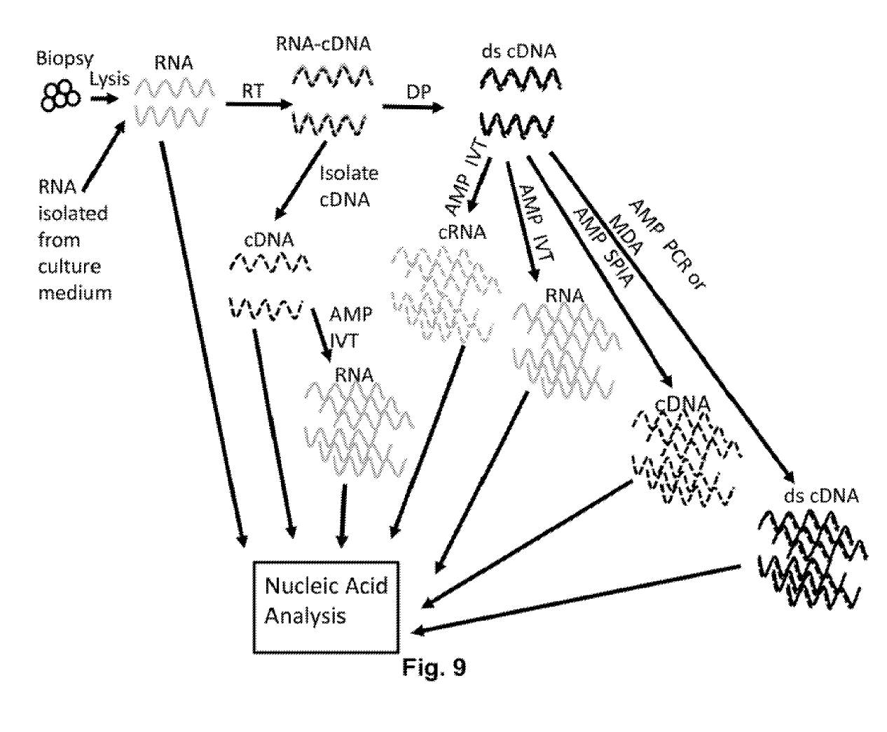

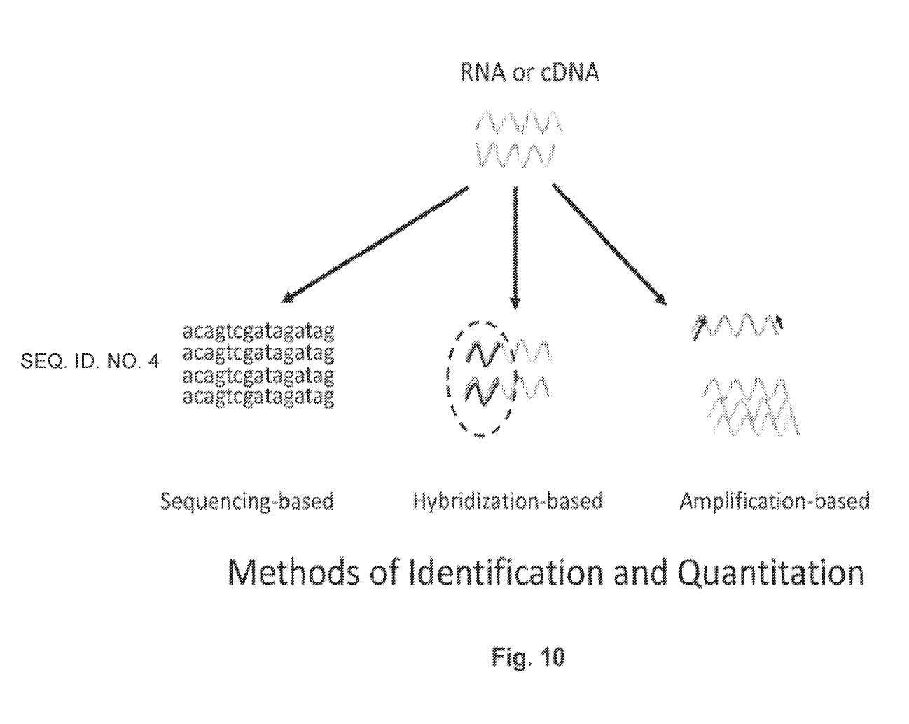

Any suitable method that can be used to identify and quantitate the expression levels of one or more transcripts can be used according to the disclosure. In some cases, expression levels of multiple transcripts can be evaluated simultaneously. In some cases, a method that can evaluate all or a large percentage of transcripts in a sample can be used. Analyses can be performed on RNA or a variety of derivative nucleic acids (see e.g., FIG. 9). In some cases, the nucleic acids can be amplified to produce sufficient nucleic acid for the method(s) used for detection and quantitation. Methods for detection and quantitation of nucleic acids include but are not limited to massively parallel sequencing (e.g., RNA-Seq), hybridization-based (e.g., microarrays) or amplification-based methods (e.g., quantitative or digital PCR) (see e.g., FIG. 10). Described below are various means for handling samples, preparing RNA, generating nucleic acid samples for analysis and generating raw data.

### V.A. Cell Treatment and Lysis

In cases in which a sample containing cells is obtained, cells can be lysed to release RNA. In some cases, such as when cell-free RNA or a lysate is obtained, no lysis step can be involved. Any suitable method for preparing cell samples for processing for transcriptome analyses can used in the compositions and methods described herein. In some cases, an entire cell sample can be immediately processed for downstream analysis. In other cases, a cell sample is processed before proceeding with molecular diagnostics. In some cases, a cell sample is divided, or cells are dissociated so that more than one sample can be derived from a biopsy. In other cases, the cells can be cultured so that more cellular material can be available for analysis. In some cases, the cells can be exposed to growth factors to promote growth. In other cases, nucleic acids can be introduced into the cells to promote growth in culture. Further, the entire or a portion of a biopsy sample can be cryopreserved so that cells can be revived and/or cultured at a later time.

In some cases, a sample of cells can be treated to facilitate the isolation of specific subspecies of RNAs using cross linking agents such as ultraviolet light or chemicals. In other cases, samples can be exposed to BrdU to facilitate isolation of recently synthesized RNA.

In some methods, a cell sample can be washed one or more times in a solution to remove unwanted components from the culture or biopsy medium and/or extraneous nucleic acids. In some cases, a solution devoid of nucleases and/or extraneous nucleic acids, that does not stress the cells, and that facilitates handling of a sample, can be used. In some cases, the solution is phosphate-buffered saline containing about 5 mg/ml of molecular biology grade bovine serum albumin. A sample can be washed by transferring the sample to one or more drops of wash solution under oil using a pipette with an inner diameter close to the size of the biopsy sample (e.g., in the 1-5 micron range) and drawing the sample in and out of the pipet several times. Other means of exposing the sample to wash solution can be used.

In cases in which a sample from an embryo comprises cells, the cells can be lysed to release nucleic acid, e.g., RNA. In some cases, cells can be lysed in a hypotonic solution containing a weak detergent, one or more RNAse inhibitors as mentioned above and a sufficiently large volume to dilute cellular constituents. One such protocol is to place a biopsy sample in hypotonic lysis buffer containing of 1-2 microliters of 0.2% Triton X-100 and RNase inhibitors in RNase free water. Any solution that facilitates lysis and allows for downstream processing and analyses can be used. Lysates can then be frozen or immediately processed for transcriptome analysis. Samples to be frozen can be rapidly cooled by submerging a container comprising the sample in liquid nitrogen and then storing the container at −80° C. or colder temperatures until subsequent processing.

In some cases, other methods can be used to lyse cells (see e.g., Brown and Audet (2008) Journal of The Royal Society Interface 5: S131-S138, incorporated by reference herein). Methods can include use of a hypotonic solution, one or more detergents (e.g. SDS, NP40, Tween, Triton X-100) at one or more different concentrations , low or high pH (e.g., pH below 6, 5, 4, 3, or 2, or pH above 8, 9, 10, 11, 12, 13), other lysis-inducing chemicals (e.g., chaotropic salts such as guanidinium isothiocyanate), enzymes (e.g., proteinase K), freeze-thaw cycles, heat (e.g., exogeneous heat from a conductor, heated solution or laser), mechanical disruption (e.g., contact with sharp object or sonication), electroporation or any combination of the aforementioned approaches. A kit such as CellsDirect (Invitrogen) and Cells-to-CT (Applied Biosystems) can be used with the compositions and methods of this disclosure.

### V.B. RNA Purification and Preparation

In some cases, a cell lysate or RNA sample can be used directly for sequencing or subsequent processing steps. In other cases, total RNA or subclasses of RNA can be isolated before sequencing or processing. The compositions and methods of the disclosure provide for any suitable methods of RNA isolation and purification that are compatible with subsequent transcriptome analysis.

In cases in which lysates are used, the lysate can be treated with a heat labile DNAse (e.g., HL-dsDNase (ArcticZymes)) to degrade DNA present in the sample before further processing.

Any commercially available method for purifying total RNA from a small number of cells that is compatible with downstream transcriptome analyses can be used. In some cases, RNA can be isolated using commercially available kits such as those provided by companies such as Arcturus, Sigma Aldrich, Life Technologies, Promega, Affymetrix, IBI or the like. Kits and protocols can also be non-commercially available. In some cases methods can use a silica-gel membrane, trizol, phenol:chloroform or other standard lab methods for RNA isolation.

In other compositions and methods, a subset of species of RNA can be isolated or selected for subsequent processing. Since ribosomal RNAs (rRNA) can constitute >80% of transcripts within cells, some methods can reduce the amount of these sequences present in the sample. In some cases, hybridization methods can be used either to deplete rRNA sequences or to select for polyadenylated RNA, which mainly consists of messenger RNA (mRNA). In some cases, rRNA can be depleted by hybridization with biotin labeled oligonucleotide probes and subsequently removed using streptavidin-coated magnetic beads, e.g., as provided by commercially available kits such as RiboMinus kit (Invitrogen) or Ribo-Zero (Epicentre). In other cases, polyadenylated RNA can be selected using oligo-dT probes, e.g., linked to substrates or beads, e.g., in columns. In other cases, rRNA can be removed through selective degradation. Since rRNA has exposed 5′ phosphates (in contrast to mRNA that has a capped 5′ end), rRNA molecules can also be removed by using an exonuclease able to specifically degrade RNA molecules bearing a 5′ phosphate such as provided by the mRNA ONLY kit (Epicentre). rRNA can also be degraded using cDNAs complementary to rRNAs and a duplex-specific nuclease (DSN). In some cases, affinity columns or tags can be used to isolate specific RNAs.

In other cases, select sequences within the transcriptome can be enriched through the use of targeted capture techniques. In some cases, the targeted capture technique can comprise incubating the lysate with primers of target sequences that are immobilized to a substrate, washing away unbound RNA and then retrieving target sequences. Target capture of RNA sequences can be performed using a number of commercially available kits including, but not limited to, Agilent's SureSelect system and Illumina's TruSeq system.

In other cases, immunoprecipitation can be used to isolate RNAs that have been cross-linked to specific proteins using methods described above (see e.g., Churchman and Weissman (2011) Nature 469: 368-375; Ingolia, et al. (2009) Science 324: 218-223; Licatalosi, et al. (2008) Nature 456: 464-470, incorporated by reference herein).

In some cases, intact RNA can be used for subsequent steps. In other cases RNA can be fragmented prior to subsequent processing. RNA can be fragmented by any appropriate means including, but not limited to, elevated temperature, exposure to chemicals (e.g., metal ions), exposure to enzymes (e.g., RNases, e.g., RNase I or RNAse III) or nebulization. RNA fragmentation can reduce or eliminate secondary structures in RNA.

In some cases, adapters can be ligated to RNA prior to subsequent processing. These adaptors can facilitate reverse transcription, tagging, amplification and/or purification.

In some cases, exogenous RNAs not present in the sample can be added to the lysate or isolated RNA sample. These spike-in RNAs can improve quantitation by allowing for the efficiency of the subsequent processing steps to be assessed (e.g. ERCC RNA Spike-In Mix (Life Technologies)).

### V.C. Reverse Transcription

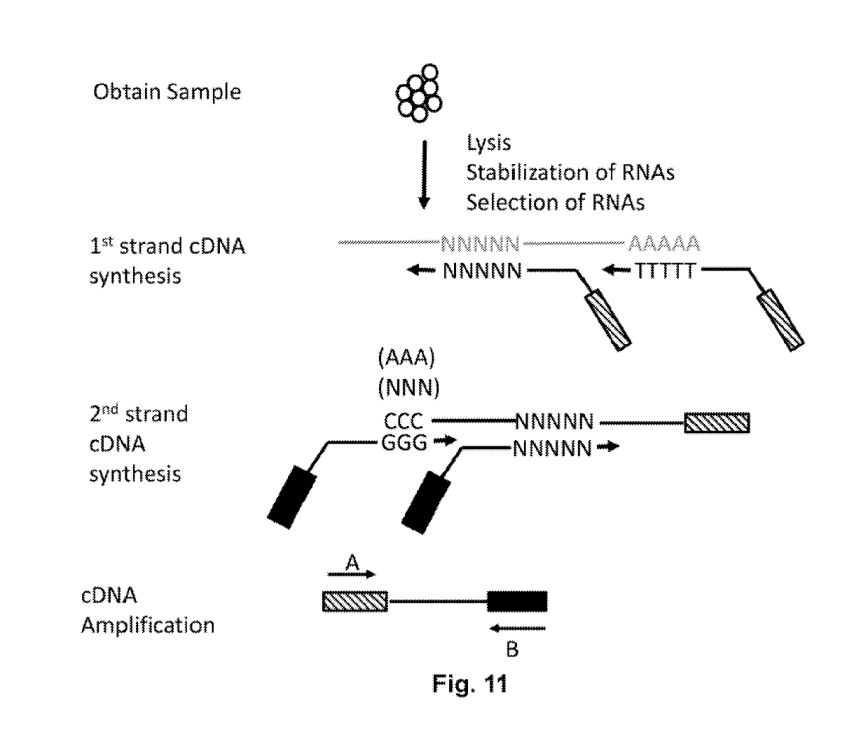

For some analytic approaches, RNA can be converted into cDNA using reverse transcriptase (see e.g., FIG. 11). Various techniques for reverse transcription are known in the art. Reverse transcription of mRNA can be primed with the use of primers that anneal to the polyadenylation sequence of transcripts (i.e., oligo-dT primers) and/or primers that anneal to other sequences within the transcript. In some cases, random primers can be used that include all permutations of the oligonucleotide. In other cases, semi-random primers can be used in which certain sequences, such as those that anneal to ribosomal RNAs are omitted. In other cases, primers with specific sequences can be used to reverse transcribe only specific transcripts.

In some compositions and methods of this disclosure, both the first and second strands of cDNA can be synthesized simultaneously using a template strand switching technique by adding a reaction mix directly to the sample lysate (see Zhu, et al. Biotechniques 30: 892-897, incorporated by reference herein). An oligodT primer can be used by Moloney murine leukemia virus (MMLV) reverse transcriptase to reverse transcribe the first strand. Following completion of the reverse transcription, a polycytosine tract can be added to the strand due to MMLV's terminal transferase activity. Inclusion of a primer with a sequence that is complementary to the polyC tract can allow extension of the second strand. This technique can be referred to as switch mechanism at the 5′ end of RNA templates (SMART) can (e.g., Clontech SMARTer™ Ultra Low RNA Kit). In other composition and methods, different primers and reverse transcriptases can be used to produce double stranded cDNA by template switching.

Double-stranded cDNA can also be produced using a protocol that uses a reverse transcriptase without terminal transferase activity. In this case, a poly(dT)-tailed primer can be used to reverse transcribe RNA. The unpolymerized primer can then be degraded with exonuclease and the cDNA can be polyadenylated with terminal transferase. A poly (dT) primer can then be used to complete the second strand synthesis using DNA polymerase I. In some cases, primers containing modified nucleotides, such as locked nucleotides, can be used to enhance primer binding and increase cDNA synthesis.

In some cases, a thermostable reverse transcriptase, such as those from thermophilic viruses, can be used so that the reverse transcription reaction can be performed at increased temperature and also to facilitate a subsequent PCR amplification. In some cases, the thermostable RT is PyroPhage from Lucigen, Inc.

In other methods, primers with unique identifiers, or barcodes, can be used in the reverse transcription and/or second strand synthesis steps that allow for quantitation. Barcodes can be used to identify the source of RNA, or used as a tool to count or quantify transcripts as described herein (see e.g., Kivioja, et al. (2012) Nat Methods 9: 72-83; Shiroguchi, et al. (2012) Proc Natl Acad Sci USA 109: 1347-52, each incorporated by reference herein). Nucleic acids from at least 2, 5, 10, 15, 25, 50, 75, or 100, 200, 300, 400, 500, 600, 700, 800, 900, or 1000 samples can be barcoded and pooled. In other applications, cDNA can be synthesized by ligating adapters to the RNAs to serve as primer annealing sites. Random primers can also be used to prime the reverse transcription throughout an RNA. In some cases, a primer mix can be semi-random with primers binding to certain sequences such as rRNAs

In some cases, other methods can be used to preserve strand information in order to determine which strand of DNA in the genome was transcribed to generate the transcript of interest. Directional, strand-specific information can be used for annotation of the transcriptome and for identifying antisense transcription. In some cases, different adaptors sequences can be attached in known orientations relative to the 5′ and 3′ ends of the RNA transcript. These protocols can generate a cDNA library flanked by two distinct adaptor sequences, marking the 5′ end and the 3′ end of the original mRNA. In other cases, one strand can be marked by chemical modification, either on the RNA itself by bisulfite treatment or during second-strand cDNA synthesis followed by degradation of the unmarked strand (as described by, e.g., Levin, et al. (2010) Nat Methods 7: 709 -715, incorporated by reference herein).

In some cases, only a single-stranded cDNA is synthesized as a substrate for amplification. In the case of in vitro transcription (iVT) based amplification methods, specific binding and initiation sites can be introduced such as 5′ extensions corresponding to one of the phage RNA polymerase priming and recognition sites. In some cases, a polynucleotide tract can be added to a cDNA to facilitate PCR-based amplification. In some cases, cDNA can be fragmented or digested to allow for sequencing of one end of the cDNA (see e.g., Hashimshony, et al. (2012) Cell Reports 2: 666-673; Islam, et al. (2012) Nat Protoc 7: 813-828., each incorporated by reference herein).

In some cases, reverse transcription reaction can be used to directly sequence RNAs. In some cases, a single molecule sequencing system such as the Helicos system described by Ozsolak and Milos ((2011) Wiley Interdisciplinary Reviews-Rna 2: 565-570, incorporated by reference herein) can be used. Other systems capable of single molecule sequencing system can be modified to sequence unamplified RNA, including the single molecule sequencing system of Pacific Biosciences and nanopore sequencing (Oxford Nanopore Technologies). RNA sequencing can also be performed using RNA polymerases that use RNA as a template. These include a number of cellular and viral polymerases that are termed RNA dependent RNA polymerases or RNA directed RNA polymerases as described by Wassesenegger and Krczal ((2006) Trends Plant Sci 11: 142) and Maida et al ((2011) Biol Chem 392: 299-304), incorporated herein by reference).

In some cases, reverse transcription reaction can be used to generate one of more copies of each cDNA that can then be sequenced. In one example of the technique, referred to as on-flow cell reverse transcription sequencing (FRT-Seq), fragmented and adaptor-ligated RNA can be placed in an Illumina flow cell containing appropriate bound primers and reverse transcriptase to generate clusters of cDNAs by bridging amplification (e.g., as described by Mamanova and Turner (2011) Nat Protoc 6: 1736-47, incorporated by reference herein).

In some cases, the cDNA rather than the RNA can be sequenced. Any of the methods described herein for single molecule sequencing can be used, e.g., single molecule sequencing systems developed by Helicos, Pacific Biosciences and Oxford Nanopore technologies.

### V.D. Nucleic acid Amplification

In some cases, nucleic acid (e.g., RNA or cDNA) from a sample from an embryo is amplified. Compositions and methods of this disclosure provide for any suitable methods for the amplification of RNA or products of reverse transcription, (see e.g., FIG. 9). RNA can be amplified by ligating sequences that facilitate replication by one of the RNA dependent RNA polymerases described herein.

In some cases, cDNA can be amplified by the use of primer binding sequences that can be added to the ends of the cDNA to serve as priming sites for amplification by PCR as shown, e.g., in FIG. 11. PCR-based amplification can be performed using any suitable method known in the art (see e.g., U.S. Pat. Nos. 4,683,195; and 4,683,202; PCR Technology: Principles and Applications for DNA Amplification, ed. H. A. Erlich, Freeman Press, NY, N.Y., 1992).

In some cases, all cDNAs are amplified. In other cases, only a subset of cDNAs is amplified. In some cases, the subset is randomly selected. In other cases, the cDNAs for amplification are specifically selected.

Suitable methods for amplification can use different primers, thermoresistant polymerases and/or amplification solutions (buffer, dNTPs, and additional reagents that can improve the amplification reaction). For example, evaluation of locus expression involving amplification of the 5′fragments of cDNAs using universal primers can be performed as described by Islam et al. ((2012) Nat Protoc 7: 813-828, incorporated by reference herein). In some cases, quasi-linear preamplification referred to as multiple annealing and looping-based amplification cycles (MALBAC) can also be applied to amplifying cDNAs (e.g., as described by Zong, et al. (2012) Science 338: 1622-6, incorporated by reference herein).

Compositions and methods of this disclosure can use any other method for amplifying nucleic acids to amplify transcribed sequences present in embryo biopsy samples (for review of amplification techniques, see e.g., Wang, et al. (2009) Nat Rev Genet 10: 57-63 and Nygaard and Hovig (2006) Nucleic Acids Research 34: 996-1014, incorporated by reference herein).

In other cases of amplifying cDNA sequences, a linear method of amplification such as in vitro transcription or single primer isothermal amplification (SPIA) (Kurn, et al. (2005) Clin Chem 51: 1973-81 and Nugen U.S. Pat. Nos. 6,692,918; 6,251,639; 6,946,251 and 7,354,717, incorporated by reference herein) can be used for amplifying cDNAs, e.g., from a single cell or small numbers of cells. Methods that combine both in vitro transcription and PCR can be used, such as the CEL-Seq method developed by Hashimshony, et al. ((2012) Cell Reports 2: 666-673, incorporated by reference herein). In this method, adapters can be ligated to the 5′ end of in vitro transcribed RNAs, the RNAs can be fragmented and another adapter can be added to the 3′ end. Those fragments containing both adapters, representing the 5′ end of RNAs, can then amplified by PCR. Since this method ligates 2 different adapters, the strandedness of the RNA that produced the clone can be determined.

Methods of nucleic acid amplification that can be used include polymerase chain reaction (PCR), ligase chain reaction (LCR) (see e.g., Wu and Wallace (1989) Genomics 4:560, Landegren et a (1988) Science 241: 1077 ; incorporated by reference herein), strand displacement amplification (SDA) (see e.g., U.S. Pat. Nos. 5,270,184; and 5,422,252, incorporated herein by reference), transcription-mediated amplification (TMA) (see e.g., U.S. Pat. No. 5,399,491, incorporated herein by reference), linked linear amplification (LLA) (see e.g., U.S. Pat. No. 6,027,923, incorporated herein by reference), self-sustained sequence replication (see e.g., Guatelli et al. (1990) Proc. Nat. Acad. Sci. USA, 87, 1874 and WO90/06995, incorporated herein by reference), selective amplification of target polynucleotide sequences (see e.g., U.S. Pat. No. 6,410,276, incorporated herein by reference), consensus sequence primed polymerase chain reaction (CP-PCR) (see e.g., U.S. Pat. No. 4,437,975, incorporated herein by reference), arbitrarily primed polymerase chain reaction (AP-PCR) (see e.g., U.S. Pat. Nos. 5,413,909, 5,861,245, incorporated herein by reference) and nucleic acid based sequence amplification (NASBA) (see e.g., U.S. Pat. Nos. 5,409,818, 5,554,517, and 6,063,603, each of which is incorporated herein by reference). Other amplification methods that can be used include: Qbeta Replicase, described, e.g., in PCT Patent Application No. PCT/US87/00880, isothermal amplification methods such as SDA, described e.g., in Walker et al., (92), Nucleic Acids Res. 20(7):1691-6, incorporated herein by reference, rolling circle amplification, described e.g., in U.S. Pat. No. 5,648,245, incorporated herein by reference, exponential amplification reaction, isothermal and chimeric primer-initiated amplification of nucleic acids, signal-mediated amplification of RNA technology and balanced PCR (see e.g., Makrigiorgos, et al. ((2002) Nature Biotechnol 20:936-9, incorporated herein by reference). Other amplification methods that can be used are described in U.S. Pat. Nos. 5,242,794; 5,494,810; 4,988,617, U.S. Ser. No. 09/854,317 and US Pub. No. 20030143599, each of which is incorporated herein by reference. In some aspects DNA is amplified by multiplex locus-specific PCR. Primers can be designed in any suitable regions 5′ and 3′ to a locus of interest and segments or complete cDNA sequences of transcripts can be amplified.

In some cases, engineered thermoresistant polymerases with high processivity and fidelity (e.g., Advantage 2 Polymerase (Clontech or KAPA HiFi (KAPA Biosystems)) can be used to enhance the amplification of entire transcripts (see Ramskold, et al. (2012) Nat Biotechnol 30: 777-82 and Picelli (2013) Nature Meth 10: 1096-98, incorporated by reference herein).

In some cases, PCR can include real-time PCR, quantitative PCR, digital PCR, or droplet digital PCR.

In some cases, a subset of amplified cDNAs can be selected following amplification using various hybridization-based target sequence capture as described herein.

In cases in which the amplified nucleic acids can be quantitated by hybridization-based methods, amplification products can be labeled through the use of nucleotides that are conjugated to labels. Labels can be any molecule or compound that can be attached to one or more nucleotides and facilitate detection of the nucleic acid. A label can include a fluorophore, chemiluminescent agent, enzyme or radioactive molecule. In some cases, nucleotides can be linked to molecules that allow for indirect detection following binding of a secondary labeled molecule. Indirect labeling methods include, but are not limited to, biotin-streptavidin and antigen-antibody systems. The choice of label can depend on sensitivity, ease of conjugation with the probe, stability, and available instrumentation. In some cases, the amplification products can be labeled following the amplification procedure.

In cases in which the nucleic acids are quantitated by amplification-based methods, the initial amplification of the cDNA (a process which can be referred to as preamplification) can be restricted to amplifying only a subset of sequences (i.e., sequences that will be assayed) and the degree of amplification can be smaller, such that a limited number of amplification products are initially produced. This scenario can be achieved through various methods, such as limiting PCR amplification cycles or the use of linear amplification techniques. This preamplification can be used to generate sufficient numbers of templates to allow for numerous amplification-based assays to be run in parallel. In various embodiments employing preamplification, the preamplification can also be used to add one or more nucleotide tags to the target nucleotide sequences so that the relative copy numbers of the tagged target nucleotide sequences is representative of the relative copy numbers of the preamplification target nucleic acids in the sample. Preamplification can be carried out for about 2 to about 20 cycles to introduce sample-specific or set-specific nucleotide tags. In some cases, the annealing sequences of the primers used for preamplification can be the same as those used in the subsequent quantitative assays. In other cases, primers that bind to sequences distal to the primer binding sites for the quantitative assay can be used in a ‘nested’ amplification strategy.

Amplification of the cDNA can yield RNA (same strand as the original RNAs in the sample), complementary RNA, single stranded cDNA, single-stranded DNA from the coding strand or double-stranded cDNA (see e.g., FIG. 9).

Amplified nucleic acids can be analyzed using one of several high throughput methods to generate data that can be used to evaluate expression, e.g., massively parallel sequencing, multiplexed hybridization to probes or multiplexed amplification-based assays.

### V.E. Sample Preparation and Raw Data Generation for Sequencing-Based Transcriptome Profiling

Compositions and methods of the instant disclosure provide for sequencing of nucleic acids. Libraries can be generated to facilitate sequencing by a number of currently available massively parallel sequencing technologies, such as the HiSeq/MiSeq (Illumina), SoLiD/Ion Torrent(Life Technologies), 454 GS FLX+/GS Junior (Roche), and Complete Genomics platforms. Sequencing libraries can consist of clones containing inserts of short fragments of DNA flanked by sequences that can be used to sequence one or both ends of the insert DNA. Protocols for preparation of libraries can be involve fragmentation of input DNA, ligation of adaptors, multiplexed amplification of individual clones and sequencing of amplified clones in parallel.

V.E.i. DNA Purification.

In some embodiments, amplified cDNAs can be purified to remove unincorporated nucleotides, primer dimers, short fragments and single-stranded nucleic acids before further processing. DNAs can be purified using gel electrophoresis or a variety of substrates that bind nucleic acids. Substrates can include magnetic beads or columns with specific nucleic acid binding properties.

V.E.ii. DNA Fragmentation

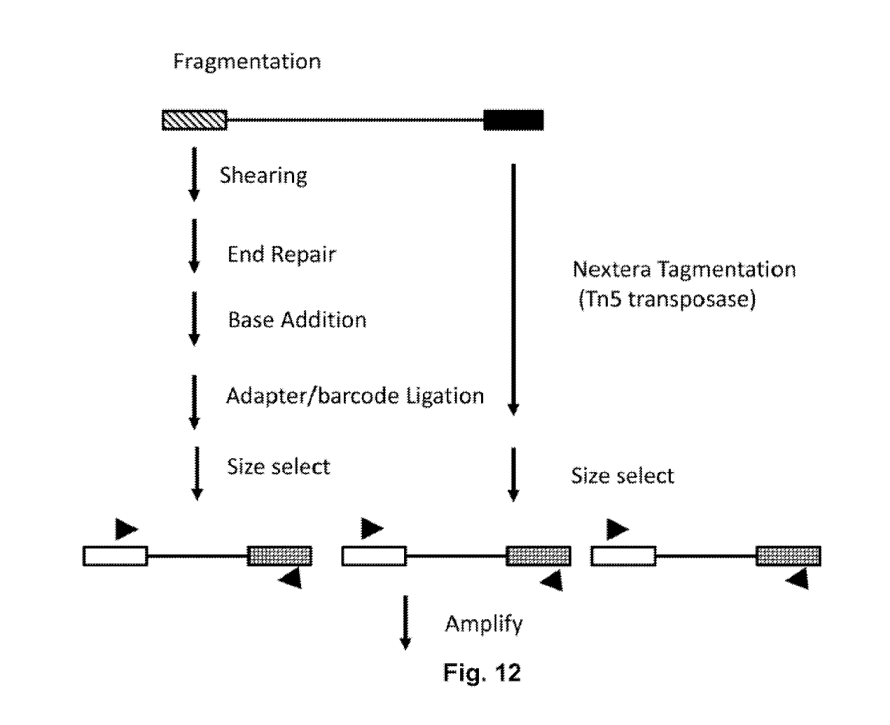

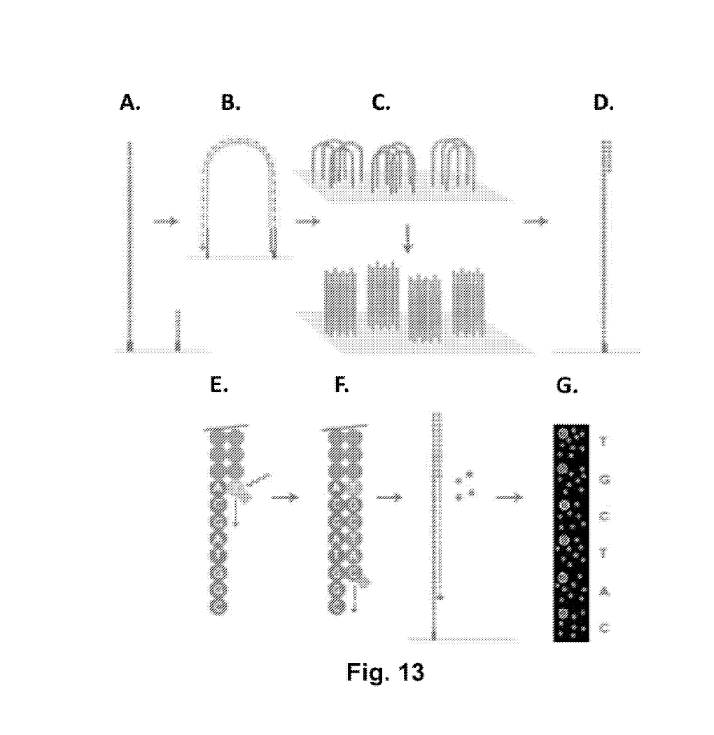

In some cases, nucleic acids can be reduced to small fragments to increase coverage from the relatively short sequence reads that can be obtained from the ends of clones using current sequencing platforms (see e.g., FIG. 12). In some cases, cDNAs can be fragmented into sizes of at least 10, 20, 30, 40, 50, 60, 70, 80, 90, 100, 200, 300, 400, 500 , 600, 700, 800, 900, 1000, 2000, 3000, 5000 base pairs in length. In some cases cDNAs can be fragmented into sizes of at most 10, 20, 30, 40, 50, 60, 70, 80, 90, 100, 200, 300, 400, 500 , 600, 700, 800, 900, 1000, 2000, 3000, 5000 base pairs in length. In some cases cDNAs can be fragmented into sizes of about 10, 20, 30, 40, 50, 60, 70, 80, 90, 100, 200, 300, 400, 500 , 600, 700, 800, 900, 1000, 2000, 3000, 5000 base pairs in length. In some cases, cDNAs can be fragmented in sizes of about 10 to about 5000 base pairs, about 10 to about 1000 base pairs, about 100 to about 5000 base pairs, or about 100 to about 1000 base pairs.

Fragmentation can be performed through physical, mechanical or enzymatic methods. Physical fragmentation can include exposing a target polynucleotide to heat or to UV light. Mechanical disruption can be used to shear a target polynucleotide into fragments of the desired range. Mechanical shearing can be accomplished through a number of methods known in the art, including repetitive pipetting of the target polynucleotide, sonication, or nebulization. Target polynucleotides can also be fragmented using enzymatic methods. In some cases, enzymatic digestion can be performed using enzymes such as using restriction enzymes.

Restriction enzymes can be used to perform specific or non-specific fragmentation of target polynucleotides. The methods of the present disclosure can use one or more types of restriction enzymes, generally described as Type I enzymes, Type II enzymes, and/or Type III enzymes. Type II and Type III enzymes can recognize specific sequences of nucleotide base pairs within a double stranded polynucleotide sequence (a “recognition sequence” or “recognition site”). Upon binding and recognition of these sequences, Type II and Type III enzymes can cleave a polynucleotide sequence. In some cases, cleavage can result in a polynucleotide fragment with a portion of overhanging single stranded DNA, called a “sticky end.” In other cases, cleavage does not result in a fragment with an overhang; rather, a “blunt end” is created. The methods of the present disclosure can comprise use of restriction enzymes that can generate either sticky ends or blunt ends.

Restriction enzymes can recognize a variety of recognition sites in the target polynucleotide. Some restriction enzymes (“exact cutters”) can recognize only a single recognition site (e.g., GAATTC). Other restriction enzymes can be more promiscuous, and can recognize more than one recognition site, or a variety of recognition sites. Some enzymes can cut at a single position within the recognition site, while others can cut at multiple positions. Some enzymes can cut at the same position within the recognition site, while others can cut at variable positions.

In some cases, Nextera kits, such as provided by Illumina/Epicentre, which use a tn5 transposase to simultaneously fragment the double-stranded DNA and ligate sequencing platform specific adaptors to the ends of the fragments, can be used. In some cases, kits such as MuSeek (Life Technologies), or other fragmentation/tag techniques can be used.

In some cases, cDNA fragmentation is not performed. In some cases, RNA molecules, before reverse transcription to cDNA, can be fragmented using any suitable method as described herein.

In some cases, fragmented DNA can be size-selected using agarose gel methods such as SizeSelect™ Gels (Life Technologies) or Pippin Prep™ kits or beads such as AMPure XP (Beckman Coulter). In other embodiments, fragmented DNA can be end repaired and/or polynucleotide tailed for subsequent steps of library preparation.

V.E.iii. DNA Strand End Repair

In some cases, fragmentation of DNA, such as through mechanical shearing or enzymatic digestion, results in fragments with a heterogeneous mix of blunt and 3′- and 5′-overhanging ends. In some cases, the compositions and methods of the disclosure provide for repair of fragment ends using methods or kits (i.e. Lucigen DNA terminator End Repair Kit) known in the art to generate ends that are designed for insertion, for example, into blunt sites of cloning vectors. In some cases, the compositions and methods of the disclosure can provide for blunt ended fragment ends of the population of DNAs sequenced. In some cases, the blunt ended fragment can be phosphorylated. The phosphate moiety can be introduced via enzymatic treatment, for example, using a kinase, (i.e. shrimp alkaline kinase).

In some cases, polynucleotide sequences can be prepared with single overhanging nucleotides by, for example, activity of certain types of DNA polymerase such as Taq polymerase or Klenow exo minus polymerase which has a nontemplate-dependent terminal transferase activity that can add a single deoxynucleotide, for example, deoxyadenosine (A) to the 3′ ends of, for example, PCR products. Such enzymes can be utilized to add a single nucleotide ‘A’ to the blunt ended 3′ terminus of each strand of the target polynucleotide duplexes. Thus, an ‘A’ can be added to the 3′ terminus of each end repaired duplex strand of the target polynucleotide duplex by reaction with Taq or Klenow exo minus polymerase, whilst the adaptor polynucleotide construct can be a T-construct with a compatible ‘T’ overhang present on the 3′ terminus of each duplex region of the adaptor construct. This end modification can also prevent self-ligation of both adapter and target such that there is a bias towards formation of the combined ligated adaptor-target sequences.

V.E.iv. Library Production and Sequencing

Numerous methods of sequence determination are compatible with the methods and systems of the described herein. Exemplary methods for sequence determination include (1) hybridization-based methods, such as disclosed in Drmanac, U.S. Pat. Nos. 6,864,052; 6,309,824; and 6,401,267; and Drmanac et al, U.S. patent publication 2005/0191656, which are incorporated by reference, (2) sequencing by synthesis methods, e.g., Nyren et al, U.S. Pat. Nos. 7,648,824, 7,459,311 and 6,210,891; Balasubramanian, U.S. Pat. Nos. 7,232,656 and 6,833,246; Quake, U.S. Pat. No. 6,911,345; Li et al, Proc. Natl. Acad. Sci., 100: 414-419 (2003), (3) pyrophosphate sequencing as described in Ronaghi et al., U.S. Pat. Nos. 7,648,824, 7,459,311, 6,828,100, and 6,210,891 and (4) ligation-based sequencing determination methods, e.g., Drmanac et al., U.S. Pat. App. No. 20100105052, and Church et al, U.S. Pat. App. Nos. 20070207482 and 20090018024.

The methods described herein can use one or more next-generation sequencing techniques to sequence nucleic acids from embryos. Next-generation sequencing techniques include, for example, Helicos True Single Molecule Sequencing (tSMS) (Harris T. D. et al. (2008) Science 320:106-109, incorporated herein by reference); 454 sequencing (Roche) (Margulies, M. et al. (2005) Nature, 437, 376-380, incorporated herein by reference); SOLiD technology (Applied Biosystems); SOLEXA sequencing (Illumina); single molecule, real-time (SMRT™) technology of Pacific Biosciences; nanopore sequencing (Soni G V and Meller A. (2007) Clin Chem 53: 1996-2001, incorporated herein by reference); semiconductor sequencing (Ion Torrent/Life Technologies; Personal Genome Machine); DNA nanoball sequencing; sequencing using technology from Dover Systems (Polonator), and technologies that do not require amplification or otherwise transform native DNA prior to sequencing (e.g., Pacific Biosciences and Helicos), such as nanopore-based strategies (e.g. Oxford Nanopore, Genia Technologies, and Nabsys).

In some cases, a library can be prepared for sequencing using an Illumina platform, comprising limited-cycle PCR in which a four-primer reaction adds bridge PCR (bPCR)-compatible adaptors to the core library (used for binding fragments to the flow cell). By including different Illumina compatible barcodes between the downstream bPCR adaptor and the core sequencing library adaptor in sets of up to 4 samples, or up to 12 samples can be run on the same flow cell. A library can be produced, size selected and quality confirmed, and combinations of 12 samples with appropriate barcodes (12-plex/flow cell) can are added to flow cells for cluster formation using a cBot (an automated system that can create clonal clusters from single molecule DNA templates). In this process, single molecules from the library can bind to one of two oligonucleotides complementary to the different adapter sequences on the flow cell surface. Through repeated annealing and extension reactions of bridged sequences, clusters of around 1000 copies of the original library molecule can be formed on a flow cell substrate (Illumina (10) Technology Spotlight: Illumina Sequencing). In some cases there can be one or more clean-up steps to remove unligated adapters.

In other cases, library production and amplification can utilize the ligation of different adapters and PCR amplification under different conditions to generate a library for sequencing on other platforms. For example, individual library clones (single DNA molecules) can be bound to beads and each bead can be encapsulated in an aqueous droplet of PCR-reaction-mixture in oil, also known as emulsion PCR. The amplicons produced can bound to the bead, thereby greatly increasing the number of copies bound to each bead. Such methods can be provided commercially, such as methods and kits sold by 454/Roche and SOLiD/Applied Biosystems. The primers used for the adaptors and sequencing can be specific to each sequencing platform.

Sequence information can be determined using methods that determine many (typically thousands to billions) of nucleic acid sequences in an intrinsically parallel manner, where many sequences can be read out preferably in parallel using a high throughput process. Such methods include but are not limited to pyrosequencing (for example, as commercialized by 454 Life Sciences, Inc., Branford, Conn.); sequencing by ligation (for example, as commercialized in the SOLiD™ technology, Life Technology, Inc., Carlsbad, Calif.); sequencing by synthesis using modified nucleotides (such as commercialized in TruSeq™ and HiSeg™ technology by Illumina, Inc., San Diego, Calif., HeliScope™ by Helicos Biosciences Corporation, Cambridge, Mass., and PacBio RS by Pacific Biosciences of California, Inc., Menlo Park, Calif.), sequencing by ion detection technologies (Ion Torrent, Inc., South San Francisco, Calif.); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods.

The amount of raw sequence data that is obtained for each sample can be determined by the number of clones sequenced, whether one or both ends of clones are sequenced, and the length of sequence reads. The amount of sequence data can impact the resolution of this approach for detecting CNVs. In some cases, only single end sequencing is performed. In other cases, paired-end sequencing is performed. The length of sequence reads can be more than 50, 100, 200, 300, 400, 500, 1000, 2000, 5,000 or 10,000 basepairs. The number of clones sequenced can be more than 1, 2, 5, 10, 20, 30, 40, 50, 60, 70, 80, 100 million.

In some embodiments, the next generation sequencing technique is 454 sequencing (Roche) (see e.g., Margulies, M et al. (2005) Nature 437: 376-380, incorporated herein by reference). 454 sequencing can involve two steps. In the first step, DNA can be sheared into fragments of approximately 300-800 base pairs, and the fragments can be blunt ended. Oligonucleotide adaptors can then ligated to the ends of the fragments. The adaptors can serve as sites for hybridizing primers for amplification and sequencing of the fragments. The fragments can be attached to DNA capture beads, e.g., streptavidin-coated beads using, e.g., Adaptor B, which can contain 5′-biotin tag. The fragments can be attached to DNA capture beads through hybridization. A single fragment can be captured per bead. The fragments attached to the beads can be PCR amplified within droplets of an oil-water emulsion. The result can be multiple copies of clonally amplified DNA fragments on each bead. The emulsion can be broken while the amplified fragments remain bound to their specific beads. In a second step, the beads can be captured in wells (pico-liter sized; PicoTiterPlate (PTP) device). The surface can be designed so that only one bead fits per well. The PTP device can be loaded into an instrument for sequencing. Pyrosequencing can be performed on each DNA fragment in parallel. Addition of one or more nucleotides can generate a light signal that can be recorded by a CCD camera in a sequencing instrument. The signal strength can be proportional to the number of nucleotides incorporated. Pyrosequencing can make use of pyrophosphate (PPi) which can be released upon nucleotide addition. PPi can be converted to ATP by ATP sulfurylase in the presence of adenosine 5′ phosphosulfate. Luciferase can use ATP to convert luciferin to oxyluciferin, and this reaction can generate light that can be detected and analyzed. The 454 Sequencing system used can be GS FLX+ system or the GS Junior System.

In some embodiments, the next generation sequencing technique is SOLiD technology (Applied Biosystems; Life Technologies). In SOLiD sequencing, genomic DNA can be sheared into fragments, and adaptors can be attached to the 5′ and 3′ ends of the fragments to generate a fragment library. Alternatively, internal adaptors can be introduced by ligating adaptors to the 5′ and 3′ ends of the fragments, circularizing the fragments, digesting the circularized fragment to generate an internal adaptor, and attaching adaptors to the 5′ and 3′ ends of the resulting fragments to generate a mate-paired library. Next, clonal bead populations can be prepared in microreactors containing beads, primers, template, and PCR components. Following PCR, the templates can be denatured and beads can be enriched to separate the beads with extended templates. Templates on the selected beads can be subjected to a 3′ modification that permits bonding to a glass slide. A sequencing primer can bind to adaptor sequence. A set of four fluorescently labeled di-base probes can compete for ligation to the sequencing primer. Specificity of the di-base probe can be achieved by interrogating every first and second base in each ligation reaction. The sequence of a template can be determined by sequential hybridization and ligation of partially random oligonucleotides with a determined base (or pair of bases) that can be identified by a specific fluorophore. After a color is recorded, the ligated oligonucleotide can be cleaved and removed and the process can be then repeated. Following a series of ligation cycles, the extension product can be removed and the template can be reset with a primer complementary to the n−1 position for a second round of ligation cycles. Five rounds of primer reset can be completed for each sequence tag. Through the primer reset process, most of the bases can be interrogated in two independent ligation reactions by two different primers. Up to 99.99% accuracy can be achieved by sequencing with an additional primer using a multi-base encoding scheme.

In some embodiments, the next generation sequencing technique is SOLEXA sequencing (ILLUMINA sequencing). ILLUMINA sequencing can be based on the amplification of DNA on a solid surface using fold-back PCR and anchored primers. ILLUMINA sequencing can involve a library preparation step. Genomic DNA can be fragmented, and sheared ends can be repaired and adenylated. Adaptors can be added to the 5′ and 3′ ends of the fragments. The fragments can be size selected and purified. ILLUMINA sequence can comprise a cluster generation step. DNA fragments can be attached to the surface of flow cell channels by hybridizing to a lawn of oligonucleotides attached to the surface of the flow cell channel. The fragments can be extended and clonally amplified through bridge amplification to generate unique clusters. The fragments become double stranded, and the double stranded molecules can be denatured. Multiple cycles of the solid-phase amplification followed by denaturation can create several million clusters of approximately 1,000 copies of single-stranded DNA molecules of the same template in each channel of the flow cell. Reverse strands can be cleaved and washed away. Ends can be blocked, and primers can by hybridized to DNA templates. ILLUMINA sequencing can comprise a sequencing step. Hundreds of millions of clusters can be sequenced simultaneously. Primers, DNA polymerase and four fluorophore-labeled, reversibly terminating nucleotides can be used to perform sequential sequencing. All four bases can compete with each other for the template. After nucleotide incorporation, a laser can be used to excite the fluorophores, and an image is captured and the identity of the first base is recorded. The 3′ terminators and fluorophores from each incorporated base are removed and the incorporation, detection and identification steps are repeated. A single base can be read each cycle. In some embodiments, a HiSeq system (e.g., HiSeq 2500, HiSeq 1500, HiSeq 2000, or HiSeq 1000) is used for sequencing. In some embodiments, a MiSeq personal sequencer is used. In some embodiments, a Genome Analyzer IIx is used.

In some embodiments, the next generation sequencing technique comprises real-time (SMRT™) technology by Pacific Biosciences. In SMRT, each of four DNA bases can be attached to one of four different fluorescent dyes. These dyes can be phospholinked. A single DNA polymerase can be immobilized with a single molecule of template single stranded DNA at the bottom of a zero-mode waveguide (ZMW). A ZMW can be a confinement structure which enables observation of incorporation of a single nucleotide by DNA polymerase against the background of fluorescent nucleotides that can rapidly diffuse in an out of the ZMW (in microseconds). It can take several milliseconds to incorporate a nucleotide into a growing strand. During this time, the fluorescent label can be excited and produce a fluorescent signal, and the fluorescent tag can be cleaved off. The ZMW can be illuminated from below. Attenuated light from an excitation beam can penetrate the lower 20-30 nm of each ZMW. A microscope with a detection limit of 20 zeptoliters (10^{˜21} liters) can be created. The tiny detection volume can provide 1000-fold improvement in the reduction of background noise. Detection of the corresponding fluorescence of the dye can indicate which base was incorporated. The process can be repeated.

In some embodiments, the next generation sequencing is nanopore sequencing (See e.g., Soni G V and Meller A. (2007) Clin Chem 53: 1996-2001, incorporated herein by reference). A nanopore can be a small hole, of the order of about one nanometer in diameter. Immersion of a nanopore in a conducting fluid and application of a potential across it can result in a slight electrical current due to conduction of ions through the nanopore. The amount of current which flows can be sensitive to the size of the nanopore. As a DNA molecule passes through a nanopore, each nucleotide on the DNA molecule can obstruct the nanopore to a different degree. Thus, the change in the current passing through the nanopore as the DNA molecule passes through the nanopore can represent a reading of the DNA sequence. The nanopore sequencing technology can be from Oxford Nanopore Technologies; e.g., a GridlON system. A single nanopore can be inserted in a polymer membrane across the top of a microwell. Each microwell can have an electrode for individual sensing. The microwells can be fabricated into an array chip, with 100,000 or more microwells (e.g., more than about 200,000, 300,000, 400,000, 500,000, 600,000, 700,000, 800,000, 900,000, or 1,000,000) per chip. An instrument (or node) can be used to analyze the chip. Data can be analyzed in real-time. One or more instruments can be operated at a time. The nanopore can be a protein nanopore, e.g., the protein alpha-hemolysin, a heptameric protein pore. The nanopore can be a solid-state nanopore made, e.g., a nanometer sized hole formed in a synthetic membrane (e.g., SiNx, or S1O₂). The nanopore can be a hybrid pore (e.g., an integration of a protein pore into a solid-state membrane). The nanopore can be a nanopore with an integrated sensors (e.g., tunneling electrode detectors, capacitive detectors, or graphene based nano-gap or edge state detectors (see e.g., Garaj et al. (2010) Nature 67: 190-3, incorporated herein by reference)). A nanopore can be functionalized for analyzing a specific type of molecule (e.g., DNA, RNA, or protein). Nanopore sequencing can comprise “strand sequencing” in which intact DNA polymers can be passed through a protein nanopore with sequencing in real time as the DNA translocates the pore. An enzyme can separate strands of a double stranded DNA and feed a strand through a nanopore. The DNA can have a hairpin at one end, and the system can read both strands. In some embodiments, nanopore sequencing is “exonuclease sequencing” in which individual nucleotides can be cleaved from a DNA strand by a processive exonuclease, and the nucleotides can be passed through a protein nanopore. The nucleotides can transiently bind to a molecule in the pore (e.g., cyclodextran). A characteristic disruption in current can be used to identify bases.

In some embodiments, nanopore sequencing technology from GENIA is used. An engineered protein pore can be embedded in a lipid bilayer membrane. “Active Control” technology can be used to enable efficient nanopore-membrane assembly and control of DNA movement through the channel. In some embodiments, the nanopore sequencing technology is from NABsys. Genomic DNA can be fragmented into strands of average length of about 100 kb. The 100 kb fragments can be made single stranded and subsequently hybridized with a 6-mer probe. The genomic fragments with probes can be driven through a nanopore, which can create a current-versus-time tracing. The current tracing can provide the positions of the probes on each genomic fragment. The genomic fragments can be lined up to create a probe map for the genome. The process can be done in parallel for a library of probes. A genome-length probe map for each probe can be generated. Errors can be fixed with a process termed “moving window Sequencing By Hybridization (mwSBH).” In some embodiments, the nanopore sequencing technology is from IBM/Roche. A electron beam can be used to make a nanopore sized opening in a microchip. An electrical field can be used to pull or thread DNA through the nanopore. A DNA transistor device in the nanopore can comprise alternating nanometer sized layers of metal and dielectric. Discrete charges in the DNA backbone can get trapped by electrical fields inside the DNA nanopore. Turning off and on gate voltages can allow the DNA sequence to be read.

In some embodiments, the next generation sequencing comprises ion semiconductor sequencing (e.g., using technology from Life Technologies (Ion Torrent)). Ion semiconductor sequencing can take advantage of the fact that when a nucleotide is incorporated into a strand of DNA, an ion can be released. To perform ion semiconductor sequencing, a high density array of micromachined wells can be formed. Each well can hold a single DNA template. Beneath the well can be an ion sensitive layer, and beneath the ion sensitive layer can be an ion sensor. When a nucleotide is added to a DNA, H+ can be released, which can be measured as a change in pH. The H+ ion can be converted to voltage and recorded by the semiconductor sensor. An array chip can be sequentially flooded with one nucleotide after another. No scanning, light, or cameras can be required. In some embodiments, an IONPROTON™ Sequencer is used to sequence nucleic acid. In some embodiments, an IONPGM™ Sequencer is used.

In some embodiments, the next generation sequencing is DNA nanoball sequencing (as performed, e.g., by Complete Genomics; see e.g., Drmanac et al. (2010) Science 327: 78-81, incorporated herein by reference). DNA can be isolated, fragmented, and size selected. For example, DNA can be fragmented (e.g., by sonication) to a mean length of about 500 bp. Adaptors (Adl) can be attached to the ends of the fragments. The adaptors can be used to hybridize to anchors for sequencing reactions. DNA with adaptors bound to each end can be PCR amplified. The adaptor sequences can be modified so that complementary single strand ends bind to each other forming circular DNA. The DNA can be methylated to protect it from cleavage by a type IIS restriction enzyme used in a subsequent step. An adaptor (e.g., the right adaptor) can have a restriction recognition site, and the restriction recognition site can remain non-methylated. The non-methylated restriction recognition site in the adaptor can be recognized by a restriction enzyme (e.g., Acul), and the DNA can be cleaved by Acul 13 bp to the right of the right adaptor to form linear double stranded DNA. A second round of right and left adaptors (Ad2) can be ligated onto either end of the linear DNA, and all DNA with both adapters bound can be PCR amplified (e.g., by PCR). Ad2 sequences can be modified to allow them to bind each other and form circular DNA. The DNA can be methylated, but a restriction enzyme recognition site can remain non-methylated on the left Adl adapter. A restriction enzyme (e.g., Acul) can be applied, and the DNA can be cleaved 13 bp to the left of the Adl to form a linear DNA fragment. A third round of right and left adaptor (Ad3) can be ligated to the right and left flank of the linear DNA, and the resulting fragment can be PCR amplified. The adaptors can be modified so that they can bind to each other and form circular DNA. A type III restriction enzyme (e.g., EcoP15) can be added; EcoP15 can cleave the DNA 26 bp to the left of Ad3 and 26 bp to the right of Ad2. This cleavage can remove a large segment of DNA and linearize the DNA once again. A fourth round of right and left adaptors (Ad4) can be ligated to the DNA, the DNA can be amplified (e.g., by PCR), and modified so that they bind each other and form the completed circular DNA template. Rolling circle replication (e.g., using Phi 29 DNA polymerase) can be used to amplify small fragments of DNA. The four adaptor sequences can contain palindromic sequences that can hybridize and a single strand can fold onto itself to form a DNA nanoball (DNB™) which can be approximately 200-300 nanometers in diameter on average. A DNA nanoball can be attached (e.g., by adsorption) to a microarray (sequencing flowcell). The flow cell can be a silicon wafer coated with silicon dioxide, titanium and hexamehtyldisilazane (HMDS) and a photoresist material. Sequencing can be performed by unchained sequencing by ligating fluorescent probes to the DNA. The color of the fluorescence of an interrogated position can be visualized by a high resolution camera. The identity of nucleotide sequences between adaptor sequences can be determined.

In some embodiments, the next generation sequencing technique is Helicos True Single Molecule Sequencing (tSMS) (see e.g., Harris T. D. et al. (2008) Science 320:106-109, incorporated herein by reference). In the tSMS technique, a DNA sample can be cleaved into strands of approximately 100 to 200 nucleotides, and a polyA sequence can be added to the 3′ end of each DNA strand. Each strand can be labeled by the addition of a fluorescently labeled adenosine nucleotide. The DNA strands can then be hybridized to a flow cell, which can contain millions of oligo-T capture sites immobilized to the flow cell surface. The templates can be at a density of about 100 million templates/cm². The flow cell can then be loaded into an instrument, e.g., HELISCOPE™ sequencer, and a laser can illuminate the surface of the flow cell, revealing the position of each template. A CCD camera can map the position of the templates on the flow cell surface. The template fluorescent label can then be cleaved and washed away. The sequencing reaction can begin by introducing a DNA polymerase and a fluorescently labeled nucleotide. The oligo-T nucleic acid can serve as a primer. The DNA polymerase can incorporate the labeled nucleotides to the primer in a template directed manner. The DNA polymerase and unincorporated nucleotides can be removed. The templates that have directed incorporation of the fluorescently labeled nucleotide can be detected by imaging the flow cell surface. After imaging, a cleavage step can remove the fluorescent label, and the process can be repeated with other fluorescently labeled nucleotides until a desired read length is achieved. Sequence information can be collected with each nucleotide addition step. The sequencing can be asynchronous. The sequencing can comprise at least 1 billion bases per day or per hour.

In some embodiments, the sequencing technique can comprise paired-end sequencing in which both the forward and reverse template strand can be sequenced. In some embodiments, the sequencing technique can comprise mate pair library sequencing. In mate pair library sequencing, DNA can be fragments, and 2-5 kb fragments can be end-repaired (e.g., with biotin labeled dNTPs). The DNA fragments can be circularized, and non-circularized DNA can be removed by digestion. Circular DNA can be fragmented and purified (e.g., using the biotin labels). Purified fragments can be end-repaired and ligated to sequencing adaptors.

In some embodiments, a sequence read is about, more than about, less than about, or at least about 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 525, 550, 575, 600, 625, 650, 675, 700, 725, 750, 775, 800, 825, 850, 875, 900, 925, 950, 975, 1000, 1100, 1200, 1300, 1400, 1500, 1600, 1700, 1800, 1900, 2000, 2100, 2200, 2300, 2400, 2500, 2600, 2700, 2800, 2900, or 3000 bases. In some embodiments, a sequence read is about 10 to about 50 bases, about 10 to about 100 bases, about 10 to about 200 bases, about 10 to about 300 bases, about 10 to about 400 bases, about 10 to about 500 bases, about 10 to about 600 bases, about 10 to about 700 bases, about 10 to about 800 bases, about 10 to about 900 bases, about 10 to about 1000 bases, about 10 to about 1500 bases, about 10 to about 2000 bases, about 50 to about 100 bases, about 50 to about 150 bases, about 50 to about 200 bases, about 50 to about 500 bases, about 50 to about 1000 bases, about 100 to about 200 bases, about 100 to about 300 bases, about 100 to about 400 bases, about 100 to about 500 bases, about 100 to about 600 bases, about 100 to about 700 bases, about 100 to about 800 bases, about 100 to about 900 bases, or about 100 to about 1000 bases.

The number of sequence reads from a sample can be about, more than about, less than about, or at least about 100, 1000, 5,000, 10,000, 20,000, 30,000, 40,000, 50,000, 60,000, 70,000, 80,000, 90,000, 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000.

The depth of sequencing of a sample can be about, more than about, less than about, or at least about 1×, 2×, 3×, 4×, 5×, 6×, 7×, 8×, 9×, 10×, 11×, 12×, 13×, 14×, 15×, 16×, 17×, 18×, 19×, 20×, 21×, 22×, 23×, 24×, 25×, 26×, 27×, 28×, 29×, 30×, 31×, 32×, 33×, 34×, 35×, 36×, 37×, 38×, 39×, 40×, 41×, 42×, 43×, 44×, 45×, 46×, 47×, 48×, 49×, 50×, 51×, 52×, 53×, 54×, 55×, 56×, 57×, 58×, 59×, 60×, 61×, 62×, 63×, 64×, 65×, 66×, 67×, 68×, 69×, 70×, 71×, 72×, 73×, 74×, 75×, 76×, 77×, 78×, 79×, 80×, 81×, 82×, 83×, 84×, 85×, 86×, 87×, 88×, 89×, 90×, 91×, 92×, 93×, 94×, 95×, 96×, 97×, 98×, 99×, 100×, 110×, 120×, 130×, 140×, 150×, 160×, 170×, 180×, 190×, 200×, 300×, 400×, 500×, 600×, 700×, 800×, 900×, 1000×, 1500×, 2000×, 2500×, 3000×, 3500×, 4000×, 4500×, 5000×, 5500×, 6000×, 6500×, 7000×, 7500×, 8000×, 8500×, 9000×, 9500×, or 10,000×. The depth of sequencing of a sample can about 1× to about 5×, about 1× to about 10×, about 1× to about 20×, about 5× to about 10×, about 5× to about 20×, about 5× to about 30×, about 10× to about 20×, about 10× to about 25×, about 10× to about 30×, about 10× to about 40×, about 30× to about 100×, about 100× to about 200×, about 100× to about 500×, about 500× to about 1000×, about 1000×, to about 2000×, about 1000× to about 5000×, or about 5000× to about 10,000×. Depth of sequencing can be the number of times a sequence (e.g., a genome) is sequenced. In some embodiments, the Lander/Waterman equation is used for computing coverage. The general equation can be: C=LN/G, where C=coverage; G=haploid genome length; L=read length; and N=number of reads.

V.E.v. Automation of Library Preparation

A number of methods can be used to automate preparation of libraries. For example microfluidic workstations, e.g., as provided by Fluidigm, Inc. can aid in automation of workflow for the SMARTer platform for cDNA amplification and generation of libraries suitable for Illumina sequencing. In some cases, the Mondrian system can be used to automate many of the steps for SPIA-based amplification protocols provided by Nugen, Inc.

### V.F. Sample Preparation and Raw Data Generation for Hybridization-Based Transcriptome Profiling

In some cases, RNA, cDNA, or amplified nucleic acids (i.e., RNA, cRNA, ss DNA, ss cDNA, ds cDNA) can be analyzed using hybridization-based methods. For some of these methods, labelled cDNAs can be hybridized with probes using stringent conditions that favor highly specific annealing (i.e., favoring perfect or close to perfect matches). Following hybridization, the probes can be washed under stringent conditions to remove unannealed and/or poorly annealed target sequences, and then target sequences that remain annealed can be detected.

V.F.i. Expression Arrays

In some cases, hybridization-based transcriptome profiling can be performed using a microarray. In general, RNA-seq and expression microarray analysis results can be highly correlated. For microarray analysis, RNA can be isolated and amplified using the same general approaches as described for RNA-Seq. The nucleic acids can be labeled during or after the amplification process. There are several commercially available kits that can perform both cDNA amplification and labeling of products: Ovation (Nugen), Message Amp (Ambion), Small sample target labeling (Affymetrix) and Bioarray small sample amplification (Enzo). In some embodiments, nucleic acids from another sample with a known genotype can be labeled with a different label so that the two samples can be competitively hybridized to allow for direct comparisons of expression between the samples on 2-channel array platforms. The reference sample can be derived from one or more cells or embryos with defined genotype(s).

Following amplification, the nucleic acid can be hybridized to a microarray. Expression microarrays can contain thousands of probes that can be complementary to known transcribed sequences that have been affixed to a substrate at defined locations. Microarrays can be printed, in situ-synthesized, high density bead or electronic and suspension bead microarrays. Arrays can contain probes that detect all or a subset of transcripts from a sample. In some cases, probes can be used that anneal to regions of transcripts that do not contain polymorphisms to facilitate assessment of expression at the locus level. In other cases, probes that specifically anneal to alleles of polymorphisms such as single nucleotide polymorphisms (SNPs) that correspond to different alleles of the loci can be used. Microarray platforms can be from commercial sources such as Affymetrix, Illumina, Roche NimbleGen or Agilent. Custom made arrays that contain user defined probes can also be used. In some instances such as Illumina and Affymetrix platforms, amplified, labeled sample nucleic acid is hybridized to the array. With other platforms such as Roche NimbleGen and Agilent, the sample can be cohybridized with a differently labelled reference sample. Following hybridization, the microarrays can be washed and scanned and the intensity values for all probes can be recorded, also according to known protocols. The raw data from the scanned microarrays can be measurements of signal intensities for the arrayed probes.

V.F.ii. Other Hybridization-Based Methods

In other embodiments, hybridization of probe and targets can be performed in solution rather than on an array. Hybridization between probe and target sequences in solution can be detected. Detection can make use of nano- or micro-particles. The particles can be encoded in a number of ways to allow for indexing. Any method that can be used to specifically encode particles can be used, e.g., employing optical/spectral codes, graphical/patterned codes, shapes or compositions. The particles can be directly linked to probes or used in a secondary step for detection. This secondary step can follow a solution-based sequence specific enzymatic reaction to determine the target genotype followed by capture onto the solid microsphere surface for detection. Reactions that can be used include allele-specific primer extension (ASPE), oligonucleotide ligation assay (OLA) and single base chain extension (SBCE). Commercial kits to employ any of these approaches can be available through Luminex, Inc using their spectrally encoded bead system (Duncan, et al. (2008) 67th Annual Meeting of the Society-for-Developmental-Biology 312, incorporated herein by reference). The protocols for such assays can be developed or modified to identify and quantitate the presence of numerous sequences.

In other embodiments, probes are labeled directly or indirectly to facilitate detection following hybridization in solution. The nucleic acids can be labeled in any way that facilitates detection including optical, sequence or mass-related properties. Nanostring technology can use unique single stranded DNA tag regions hybridized to RNA probes labeled with specific fluorophores to provide spectral barcoding that can be detected at the single molecule level using optical microscopy (see e.g., Geiss, et al. (2008) Nat Biotechnol 26: 317-25, incorporated herein by reference). DNA barcodes attached to probes can allow solution-based hybridization, and read-out can be through sequencing or chip arrays. MassCode technology can use probes that have distinct molecular weight tags that can be released by UV exposure (see e.g., Richmond, et al. (2011) Plos One 6: e18967, incorporated by reference). A variety of labeling and detection methods can be used to identify probes that have annealed to target sequences for the application in this disclosure.

In cases in which a hybridization-based method is used, the number of targets that are assayed can vary from only one target sequence to one from each chromosome to identify whole chromosomal aneuploidies (i.e., 24 target sequences) to more than thousands. More target sequences can enhance the sensitivity, specificity and resolution of these assays. The number of target sequences can be more than 24, 50, 100, 200, 500, 1000, 5000, 10,000, 50,0000, 100,000, 500,0000 or 1,000,000.

### V.G. Sample Preparation and Raw Data Generation for Amplification-Based Transcriptome Profiling

In some cases, methods for identifying and quantitating transcript levels can be performed using an amplification-based method. In some cases, the amplification method can be PCR. For a review of PCR methods, protocols, and principles in designing primers, see, e.g., Innis, et al., PCR Protocols: A Guide to Methods and Applications, Academic Press, Inc. N.Y., 1990. There are at least two general amplification-based approaches that can be used to determine an amount of template in a sample: quantitative amplification and digital amplification.

V.G.i. Quantitative Amplification

Quantitative amplification can be used to determine the amount of template based on the number of cycles of amplification to cross a threshold of detection. In some cases, this type of quantitation can be performed using PCR as the method of amplification. A guideline of steps for experimental design and data analysis for quantitative PCR (qPCR) analyses is outlined by Bustin, et al. ((2009) Clinical Chemistry 55: 611-622, incorporated herein by reference). In some cases, qPCR comprises monitoring the amount of amplification product in real time. In some cases, fluorescence-based technologies can be used, e.g.,(i) probe sequences that fluoresce upon nuclease-catalyzed hydrolysis (TaqMan; Applied Biosystems, Foster City, Calif., USA) or hybridization (LightCycler; Roche, Indianapolis, Ind., USA); (ii) fluorescent hairpins; or (iii)intercalating dyes (SYBR Green).

Fluorogenic nuclease assays are one example of a real-time quantification method that can be used successfully in the methods described herein. This method of monitoring the formation of amplification product can involve the continuous measurement of PCR product accumulation using a dual-labeled fluorogenic oligonucleotide probe (“TaqMan®) (see e.g., U.S. Pat. No. 5,723,591; Heid et al., 1996, Heid, et al. (1996) Genome Research 6: 986-994, incorporated herein by reference). Other detection/quantification methods that can be employed in this disclosure include (1) FRET and template extension reactions (see e.g., U.S. Pat. No. 5,945,283 and PCT Publication WO 97/22719), (2) molecular beacon detection (see e.g., Piatek et al., 1998, Nat. Biotechnol. 16:359-63; Tyagi, and Kramer, 1996, Nat. Biotechnology 14:303-308; and Tyagi, et al., 1998, Nat. Biotechnol. 16:49-53), (3) Scorpion detection (see e.g., Thelwell et al. 2000, Nucleic Acids Research, 28:3752-3761 and Solinas et al., 2001, Nucleic Acids Research 29:20), (4) Invader detection (see e.g., Neri, B. P., et al., 2000, Advances in Nucleic Acid and Protein Analysis 3826: 117-125 and U.S. Pat. No. 6,706,471) and (5) padlock probe detection (see e.g., Landegren et al., 2003, Comparative and Functional Genomics 4:525-30; Nilsson et al., 2006, Trends Biotechnol. 24:83-8; Nilsson et al., 1994, Science 265:2085-8), each reference hereby incorporated in its entirety.

In some embodiments, fluorophores can be used as detectable labels for probes including, e.g., rhodamine, cyanine 3 (Cy 3), cyanine 5 (Cy 5), fluorescein, Vic™, Liz™, Tamra™, 5-Fam™, 6-Fam™, and Texas Red (Molecular Probes). Vic™, Liz™, Tamra™, 5-Fam™, 6-Fam™ are all available from Applied Biosystems, Foster City, Calif.

Devices can perform a thermal cycling reaction with compositions that can contain a fluorescent indicator, a source that emits a light beam of a specified wavelength, a detection system that can quantify the fluorescence emitted and a system to display the intensity of fluorescence after each cycle. Devices comprising a thermal cycler, light beam emitter, and a fluorescent signal detector, are described, e.g., in U.S. Pat. Nos. 5,928,907; 6,015,674; and 6,174,670, incorporated herein by reference. In some cases, each of these functions can be performed by separate devices. For example, if a Q-beta replicase reaction for amplification is employed, in some cases the reaction may not take place in a thermal cycler, but can include a light beam emitted at a specific wavelength, detection of the fluorescent signal, and calculation and display of the amount of amplification product.

In some cases, combined thermal cycling and fluorescence detecting devices can be used for precise quantification of target nucleic acids. In some cases, fluorescent signals can be detected and displayed during and/or after one or more thermal cycles, thus permitting monitoring of amplification products as the reactions occur in “real-time.” In certain embodiments, one can use the amount of amplification product and number of amplification cycles to calculate how much of the target nucleic acid sequence was in the sample prior to amplification.

According to some cases, the amount of amplification product can be monitored after a predetermined number of cycles sufficient to indicate a presence of the target nucleic acid sequence in a sample. For any given sample type, primer sequence, and reaction condition, how many cycles are sufficient to determine the presence of a given target nucleic acid can be determined. By acquiring fluorescence over different temperatures, the extent of hybridization can be followed. The temperature-dependence of PCR product hybridization can be used for the identification and/or quantification of PCR products. Accordingly, the methods described herein encompass the use of melting curve analysis in detecting and/or quantifying amplicons. Melting curve analysis is well known and is described, for example, in U.S. Pat. Nos. 6, 174,670; 6472156; and 6,569,627, each of which is hereby incorporated by reference. In illustrative embodiments, melting curve analysis can be carried out using a double-stranded DNA dye, such as SYBR Green, Eva Green, Pico Green (Molecular Probes, Inc., Eugene, Oreg.), ethidium bromide, and the like (see Zhu et al., 1994, Anal. Chem. 66: 1941 -48, incorporated herein by reference).

Primers can be validated empirically to determine amplification efficiency prior to use. In some cases, these primers can be chosen from databases or commercially available catalogs; in other cases, the primers can be custom synthesized. The number of target sequences to assays can depend upon the resolution that is desired. In some cases, only one target sequence from each chromosome can be included to identify whole chromosomal aneuploidies (i.e., 24 target sequences). In other cases, many more than 24 target sequences can be included to enhance the sensitivity, specificity and resolution of these assays. The number of target sequences can be more than 24, 50, 100, 200, 500, 1000, 5000, 10,000, 50,0000, 100,000, 500,0000 or 1,000,000.

In some cases, an internal control can be employed to quantify the amplification product indicated by the fluorescent signal. See, e.g., U.S. Pat. No. 5,736,333, incorporated herein by reference.

In certain embodiments, a preamplification step is performed prior to the qPCR to enhance the number of target sequences that can be assayed and/or to introduce tags on specific nucleic acids. Preamplification prior to qPCR can be performed for a limited number of thermal cycles (e.g., 5 cycles, or 10 cycles) to provide quantitative amplification of the nucleic acids in the reaction mixture. In certain embodiments, the number of thermal cycles during preamplification can be about 3, 4, 5, 6, 7, 8, 9, 10, 11, 12, 13, 14, 15, or more than 15. In other cases, alternative means of quantitative amplification can be used. In some cases, a preamplification step is not performed.

V.G.ii. Digital Amplification

In digital amplification, a limiting dilution of the sample can be made across a large number of separate amplification reactions such that most of the reactions can have no template molecules and can give a negative amplification result. In counting the number of positive amplification results, e.g., at the reaction endpoint, the individual template molecules present in the original sample can be counted one-by-one. In digital amplification, quantitation can be independent of variations in the amplification efficiency since successful amplifications can be counted as one molecule, independent of the actual amount of product. In some cases, an amplification method will be PCR. For discussions of “digital PCR” see, for example, Vogelstein and Kinzler (1999) Proceedings of the National Academy of Sciences of the United States of America 96: 9236-9241; McBride et al., U.S Patent Application Publication No. 20050252773, incorporated herein by reference.

In certain embodiments, a preamplification step as described above for quantitative amplification can be performed before digital quantitation. In some embodiments, a preamplification step is not performed prior to digital amplification.

For digital amplification, aliquots of the sample can be distributed to separate amplification reactions such that each individual amplification reaction can be expected to include one or fewer amplifiable nucleic acids. In some cases, a set of serial dilutions of the targets can be tested. In some cases, identical (or substantially similar) amplification reaction conditions can be run for all of the assays. In other cases, a variety of amplification conditions optimized for each individual reaction can be performed. Any amplification method can be employed, e.g., PCR, real-time PCR or endpoint PCR. Amplification products can be detected, for example, using a universal probe, such as SYBR Green, or target- and reference-specific probes, which can be included in digital amplification mixtures. In some cases, only one target sequence from each chromosome can be assayed to identify whole chromosomal aneuploidies (i.e., 24 target sequences). In other cases, many more than 24 target sequences can be included to enhance the sensitivity, specificity and resolution of these assays. The number of target sequences can be more than 24, 50, 100, 200, 500, 1000, 5000, 10,000, 50,0000, 100,000, 500,0000 or 1,000,000.

A variety of approaches and devices can be used to perform these multiplexed reactions. Digital amplification methods can make use of certain-high-throughput devices suitable for digital PCR, such as microfluidic devices typically containing a large number of small-volume reaction sites (e.g., nano-volume reactions, wells, or chambers). These reaction mixtures can be performed in a reaction/assay platform or microfluidic device or can exist as separate droplets, e.g., as in emulsion PCR. Illustrative Digital Array™ microfluidic devices are described in U.S. application Ser. No. 12/170,414, incorporated herein by reference. Methods for creating droplets having reaction component(s) and/or conducting reactions therein are described in U.S. Pat. No. 7,294,503, U.S. Patent Publication No. 20100022414, U.S. Patent Publication No. 20100092973, incorporated herein by reference. In some cases, a droplet comprising target nucleic acids and a droplet comprising reaction reagents (e.g., nucleotides, polymerase, etc.) can be merged into a single droplet. Any technology that allows for high throughput means to set up, perform and monitor amplification reactions can be used.

### VI. DETECTION OF CNAS IN THE TRANSCRIPTOME

This disclosure provides compositions and methods for detecting CNAs by several different methods that can be referred to as regional expression-based, breakpoint identification-based and expression signature-based CNA detection. An expression-based method can identify CNAs based on alterations in the expression of dosage sensitive loci or alleles in the affected genomic region. A breakpoint identification approach can look for evidence of breakpoints that can indicate a structural genomic alteration. An expression signature-based approach can look for evidence of CNAs by looking for expression profiles of loci that are associated with CNAs encompassing both primary and secondary transcriptional responses.

### VI.A. Regional Expression-Based CNA Detection (RECNAD)

For detecting CNAs in the transcriptome, one approach can be through the identification of regions of the genome or corresponding transcriptome with generally altered expression relative to one or more references. This approach can rely on the presence of a sufficient number of transcribed loci or alleles that are dosage sensitive in the genomic region(s) of interest to facilitate detection. Example 1 shows that a high percentage of transcribed loci on 3 different mouse chromosomes are dosage sensitive in preimplantation embryos. An expression-based approach can make use of accurate quantitation of transcripts produced by loci and/or alleles. To quantitate the expression from loci and/or alleles, a two-step process can be followed. First, raw expression data can be assigned to respective regions of a reference genome or transcriptome sequence to generate regional expression counts (RECs). The REC data from a sample can then be compared to a reference to identify regions of the sample's transcriptome that have patterns of altered expression that can be consistent with an alteration in copy number of the corresponding genomic region.

VI.A.i. Generating Regional Expression Count Data for Loci and Alleles from RNA-Seq Data

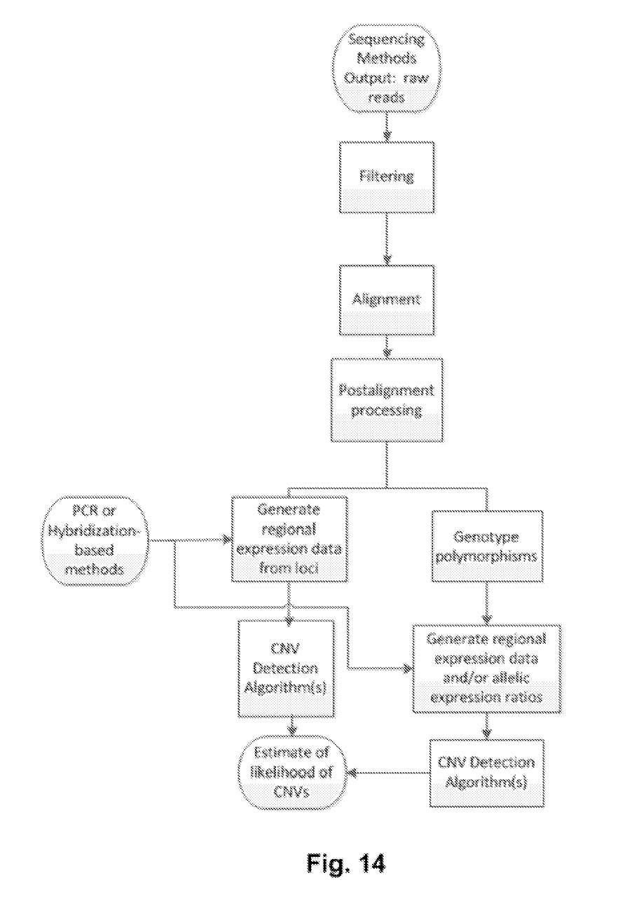

RNA-Seq can be used for generating REC data. RNA-Seq can encompass second generation or massively parallel sequencing platforms and any other high throughput methods for sequencing RNAs or derivative nucleic acids obtained from a sample. RNA-Seq can be an unbiased method, can have a large dynamic range of detection and can generate sequence data from transcribed sequences. RNA-Seq can generate raw sequence data, and several steps can be followed to convert these data into regional expression counts, including quality assessment, data filtering, sequence alignment, definition of regions, quantitation of RNA abundance in regions and normalization (see e.g., FIG. 14).

VI.A.i.a. Quality Assessment and Data Filtering

In some cases, the first analytic step after completing the sequencing run can be to evaluate the quality of raw reads and remove, trim or correct reads that do not meet the defined standards. Generally, these steps can include visualization of base quality scores (phred scores) and nucleotide distributions, trimming of reads and read filtering. Filtering of sequences can be based on sequence and/or base quality score, sequence length distribution or sequence properties including primer contaminations, overrepresented sequences, sequence duplication levels and content of N, GC and/or kmers. Quality analysis and filtering can be performed by a number of stand-alone tools including: NGSQC Toolkit, PRINSEQ, FASTQ, FASTQC FASTX-Toolkit, PIQA, TileQC. Quality analysis and filtering can also be performed as part of an analytic package such as Galaxy, HtSeqTools and Solexa QA. Sequencing reads with a base call accuracy less than 90%, 95%, 99%, 99.9%, 99.99% or 99.999% can be filtered out of the data set. In cases in which exogeneous spike-in RNAs have been added to the sample, the correlation between measured and actual copy number can also be used as a quality metric. In the case of spike-in correlations, correlation coefficients or coefficients of determination of less than 0.9, 0.8. 0.7, 0.6 or 0.5 can be used as a threshold for identifying substandard quality samples. In some cases, correlation coefficients or coefficients of determination of greater than 0.5, 0.6, 0.7, 0.8, 0.9, 0.95, 0.99, 0.999 can be used to select samples suitable for downstream analysis.

VI.A.i.b. Aligning Sequence Reads

Filtered sequence reads can be aligned to a reference genome or transcriptome sequence to generate aligned sequence reads. In some cases, a reference sequence can be a genomic sequence such as genome assemblies from GRC or NCBI. In other cases, the sequence reads can be aligned to a transcriptome assembly such as those developed by Ensembl or NCBI. In other cases, sequences can be aligned to custom reference sequences derived from a specific group or individual including one or both parents who produced the embryo being evaluated. Any program that can accurately and efficiently align RNA-Seq reads to one or more reference sequences can be used. In some programs, indexing of the reference or sample sequence is performed to reduce the computational demands of such searches. In the case of alignments of RNA-Seq data to a genome reference sequence, mapping algorithms can also identify introns. Examples of programs that can be used include TopHat, SplitSeek, SOAPals, SpliceMap, SplitSeek, QPALMA/GenomeMapper/PALMapper, Passion, RNA-Mate, RUM, SOAP Splice, Supersplat, HMMSplice, STAR (Garber, et al. (2011) Nat Methods 8: 469-477, incorporated herein by reference).

In some cases, the transcripts can be mapped to a transcriptome database such as Ensembl. For this type of mapping, any aligner that has been developed for mapping reads contiguously to a reference (i.e., not designed for reads with splice events) can be used. This technique can include the use of additional alignment software such as MAQ, BWA, PASS, SHRIMP, RMAP, SOAP2, ELAND, SeqMap, ZOOM, MOM, Vmatch, Cloudburst, AB map reads, MuMRescueLite, Novoalign, Zoom, Mosaik (Horner, et al. (2010) Briefings in Bioinformatics 11: 181-197 and Fonseca, et al. (2012) Bioinformatics 28: 3169-77, incorporated herein by reference).

Aligned sequence reads can also be used to generate a transcriptome assembly. Such programs can assemble the alignments into a parsimonious set of transcripts and can predict novel loci and isoforms according to the read mapping results on the reference genome. Examples of assembly programs are Cufflinks, G-MO.R-Se, Scripture, ERANGE Multiple-K, Rnnotator, Trans-ABySS, Oases and Trinity (Martin and Wang (2011) Nat Rev Genet 12: 671-682, incorporated herein by reference).

VI.A.i.c. Correction for Mapability

In some cases, the aligned sequences can be assessed for mapability, which can be defined as the probability for a region in the reference genome that a read originating from it is unambiguously mapped to it, Mapability can be calculated by programs such as GEM. Regions with higher mapability can have more unique sequences and produce less ambiguous reads, and vice versa. Mutations and/or sequencing errors in just one or two positions in low mapability regions can cause the reads to be mapped to wrong position. This can be especially common for repetitive regions. Different strategies can be used for dealing with multi-reads including: (1) discarding the reads; (2) choosing a random position out of all of equally good match position; (3) reporting all possible positions. The list of programs implementing mapability correction can include ReadDepth, Control-FREEC, HMMCOPY and CONSERTING (Liu(13) Oncotarget 4: 1868-81, incorporated herein by reference). Control-FREEC and CONSERTING can skip the regions with low mapability (default <0.85 and 0.9 in Control-FREEC and CONSERTING respectively), and only reads falling in high mapability regions can be used to call CNAs. HMMCOPY and OncoSNP-SEQ can correct mapability bias in read counts by dividing the raw read counts by regional mapability. To prevent overcorrection, ReadDepth can use the same formula to correct read depth data in only high mapability region (default >0.75) and can ignore the RD data in low mapability region.

VI.A.i.d. Generation of Locus Expression Counts (LREC)

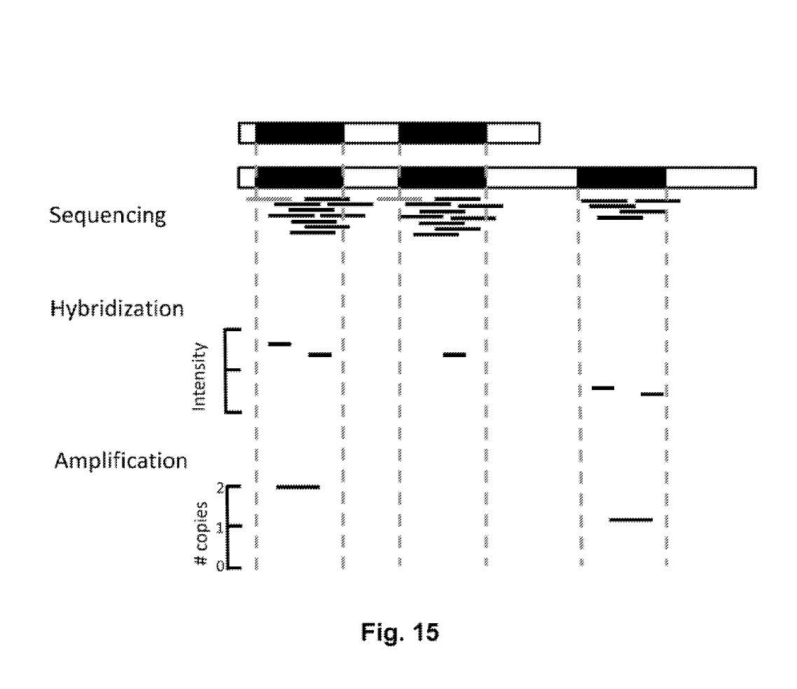

A variety of approaches can be applied to convert the aligned sequences into a dataset that presents the relative abundance of sequences within predefined regions of the transcriptome, referred to as regional expression counts (RECs) (see e.g., FIG. 15). These data can be expressed in terms of read depth, defined as the number of reads covering a predetermined region of an alignment file, or read count, the number of reads falling into a predefined region in the reference genome. In some cases, these predefined regions can be determined by biologic boundaries such as loci, isoforms of loci or exons. In other cases, these predefined windows can be specified lengths of nucleotides within each locus. Lengths of nucleotides can be single nucleotides or larger numbers of nucleotides. In some cases, combinations of more than one type of predefined region can be used. In some cases, the size of RECs can be determined by the requirements of the algorithms used in downstream analyses. Counts can be determined by summing the number of reads that begin or end within the specified window or in which a specific location within the read sequence falls within the specified window. In some cases, the REC can represent the total reads within the specified window. In other cases, RECs can represent the average of counts of subregions within the specified window (e.g., average counts for bases within an exon or average counts for exons within a transcribed locus). In some cases, the count data can be normalized to account for differences in total amount of sequence produced per sample. Two standard means of normalizing are to present the data as reads per kilo base per million (RPKM) or fragments per kilobase of transcript per million (FPKM).

In some cases, the Cufflinks program can be used to determine expression counts for loci. Cufflinks and an additional program, Cuffdiff, can implement a linear statistical model to estimate an assignment of abundance to each transcript. This estimate can explain the observed reads with maximum likelihood. Cufflinks and Cuffdiff can calculate the expression level of each alternative splice transcript of a locus and sums the expression level of each splice variant. This estimate of locus expression can be directly proportional to other techniques for measuring locus expression such as reads per kilo base per million (RPKM) or fragments per kilobase of transcript per million (FPKM). A number of other quantitation tools can be used for quantitating locus expression, such as rpkmforgenes and BEDTools.

In other cases, RECs can be determined per base. To generate depth of coverage information of each base, PILEUP files can be generated using SAMtools or BEDTools.

VI.A.i.e. Generation of Allelic Regional Expression Counts (ARECs)

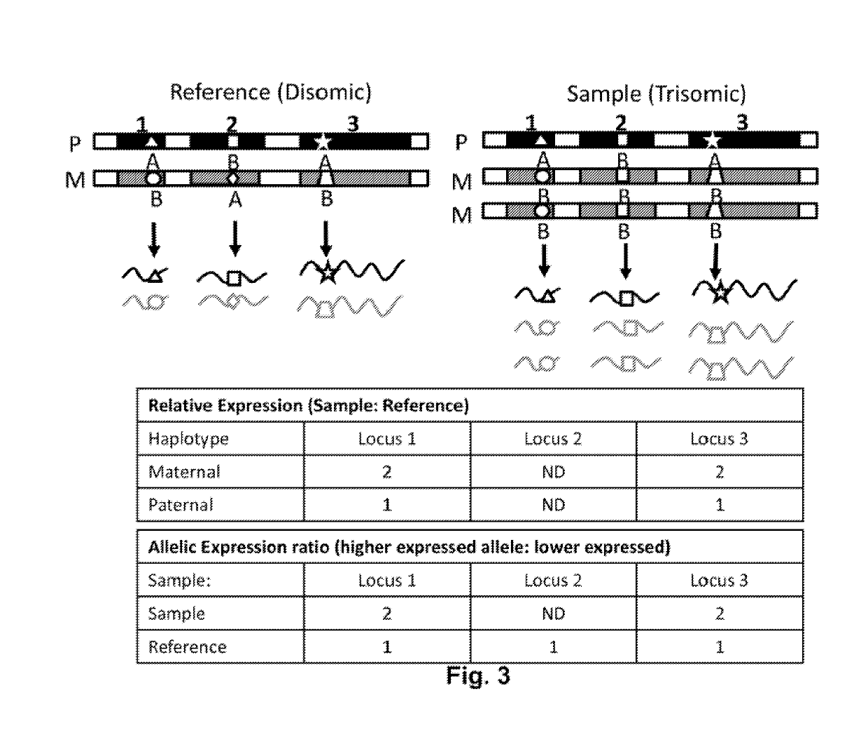

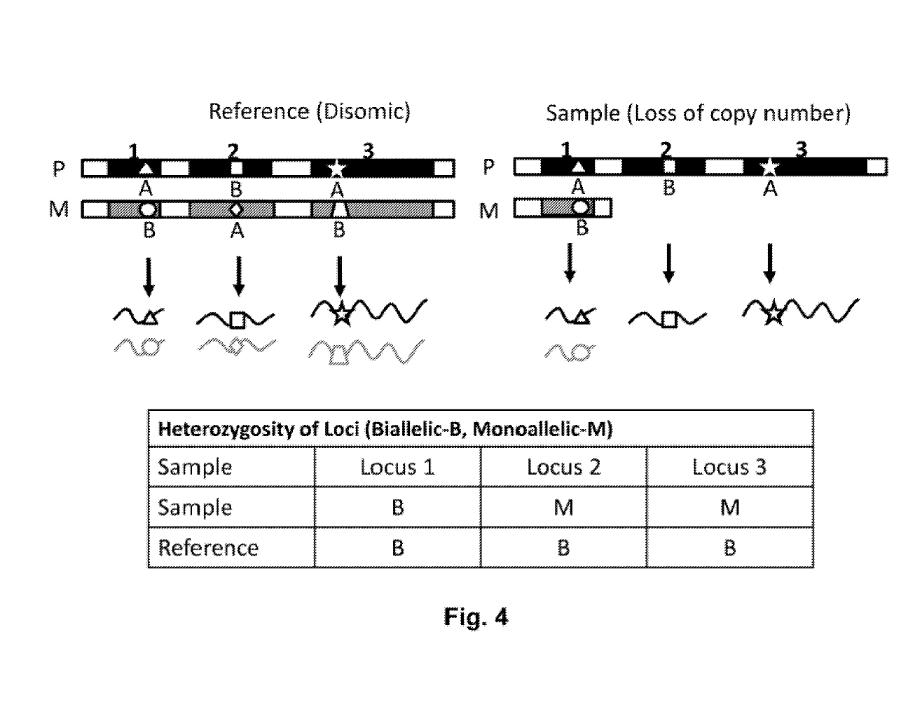

In some cases, expression counts can be generated for alleles rather than loci. To assess the expression of alleles, polymorphisms that distinguish the alleles and are present in transcripts can be evaluated (see e.g., FIGS. 3 and 4). In some cases, polymorphisms evaluated can be single nucleotide polymorphisms (SNPs), which are present in coding regions at an average frequency of about 1 every 300 basepairs within the human population. Heterozygous SNPs can allow for the absolute or relative expression of allele(s) of a locus to be determined.

To identify heterozygous SNPs, the depth of coverage for each base can be determined. This parameter can provide a confidence score for calls and can be generated by any suitable algorithm, such as SAMToo1s software. Variant sites can then be called by any algorithm that can identify and call variants. One such example is Genome Analysis Toolkit software. In some cases, software for SNP genotyping that can be used includes SOAPsnp, MAQ and Beagle.

In some cases, other polymorphic variations such as indels (small insertions or deletions) can be used to evaluate allelic expression. Generally, any type of polymorphism that is present within the transcript of interest and differs between alleles present in the sample can be used to assess allelic expression.

Once alleles have been distinguished by polymorphisms, the relative expression of each allele can be determined using any algorithm that can determine expression levels from these data such as those described herein for determining locus expression levels. Since polymorphisms have defined locations within the genome, the specified window for expression counts for alleles can be the bases involved in the polymorphism. For example, the window for a SNP can be one base pair or a larger region that encompasses the SNP. In some cases, haplotypes of polymorphisms can be determined by localizing particular alleles of a polymorphism to particular segments of chromosomal homologues. When haplotype information is present, it can be possible to determine which alleles of a polymorphism are associated with: (1) a particular allele of a locus, (2) a particular region of or an entire chromosomal homologue or (3) a parental haplotype (i.e., genetic material contributed from one parent to the sample). In the case of haplotyped polymorphisms located in the same locus, the expression of an allele can be determined by incorporating expression data from the respective alleles of all polymorphisms. In some cases, the expression data from all polymorphisms within a locus can be averaged.

VI.A.ii. Generating LREC and AREC Data from Hybridization-Based Methods

Raw expression data from hybridization methods can also be used to generate REC data (see e.g., FIG. 15). Since hybridization-based methods also can have biases due to technical aspects such as the efficiency and specificity of binding of probes and parameters of detection, data can be normalized. In some cases, data can be normalized to remove non-relevant effects such as the GC content of the target sequence, probe specific intensity bias due to differences in binding affinity and spatial artifacts. Normalization can be performed using methods that include, but are not limited to, mean-signal, spike-in or quantile normalization. In the case of hybridization-based methods, the smallest unit of expression can be defined by the size of the probe(s) in the region of interest. In cases in which more than one probe is present within the evaluated region, all probe data can be presented or all data can be compressed to a single locus value using weighted averaging or other appropriate methods.

For generating REC data from the raw expression data, the estimated expression of predetermined windows can then be tabulated using any algorithm capable of doing these calculations. Predetermined regions that can be used include the locus, isoform, exon or sequence to which the probe anneals. In cases in which probes are used that can distinguish alleles of one or more polymorphisms associated with alleles of one or more loci, then expression of alleles can be assessed. There are a variety of software packages available for hybridization-based detection methods that can genotype SNPs and provide relative intensity data for each allele. In some cases, probes can be included in the assay to assess the copy number of one or a small number of genomic loci. In other cases, probes can be included to evaluate the copy number of all chromosomes at varying degrees of resolution.

VI.A.iii. Generating LREC and AREC Data from Amplification-Based Methods

Any method that can determine transcript abundance of predefined regions of the sample's transcriptome using raw data generated by amplification-based methods for quantifying locus or allele expression can be used. The minimal predefined region can be the amplicon, but can be expanded to the level of exons, loci or specified lengths of nucleotides. The predetermined region for polymorphisms can be the variant bases.

In some cases, quantitation can be absolute, based on the use of a standard curve generated by determining threshold cycles for a range of defined concentrations of one or more control RNA. In other cases, quantitation can be relative, with results being expressed as a ratio to an external reference sample known as a calibrator. Methods for relative quantitation include, but are not limited to, the standard curve, comparative Cₜ(2^{-ΔΔCt}), Q-gene, DART-PCR, Liu and Saint method, Pfaffl et al. method and Gentle et al model as described by Wong and Medrano ((2005) Biotechniques 39: 75-85, incorporated by reference herein). Since different samples can differ in the amount of input RNA, normalization to one or more transcripts from the sample can be performed. Internal controls can be chosen from standard lists of such controls or identified empirically using methods such as those described by Bustin, et al. ((2005) Journal of Molecular Endocrinology 34: 597-601, incorporated by reference herein) and Wong and Medrano ((2005) Biotechniques 39: 75-85, incorporated by reference herein).

For digital PCR, absolute numbers of target sequence can be determined through the use of one or more standard curves generated using control samples with defined numbers of copies of target sequence.

In some cases, amplification-based assays can assess the expression of one or more loci by amplifying regions that do not contain polymorphisms. In other cases, assays can be developed that amplify only specific alleles of polymorphisms and thereby allow for quantitation of expression of particular allele(s) of a locus. In some cases, the expression of alleles from more than one locus can be evaluated by performing a multiplex assay. In some cases, the expression of only a few loci or alleles can be interrogated to assess the copy number of one or a small number of genomic regions. In some cases, a larger number of assays can be included such that the copy number of all chromosomes can be assessed.

LREC data can be generated from any of the above amplification-based expression data by assigning expression data to any of the previously described predetermined regions using the coordinates of the amplicons based on the primer annealing sequences.

VI.A.iv. Normalization of Expression Counts.

In some cases, the regional expression count data are normalized to take into account biases that may be introduced by the methods used to generate the data or the analytic methods. In some cases, the data are normalized for GC content. For RNA-Seq data, the average read depth of a bin or read count in a region can have a unimodal relationship with its GC content, regardless of the chosen biniregion size or average coverage. Bins with high or low GC-content can have lower mean read depth than bins with medium GC-content (40% to 55% GC). This phenomenon can be partially due to PCR efficiency in amplification and sequencing. Hybridization-based methods can also be affected by GC content. There are a number of means of correcting fbr GC bias such as those described by Benjamini and Speed ((2012) Nucleic Acids Res 10: E72), Teo ((2012) Bioinformatics 28: 2711-18) and Yoon ((2009) Genome Res 19: 1586-92, each incorporated herein by reference).

In some cases, batch-batch effects or other biases within the data can be removed with other methods such as principal component analysis, singular value decomposition or discrete wavelet transformation. In some cases, statistical methods can be used with no additional normalization because the samples are compared to controls generated using the same techniques. For methods where samples and controls are generated using the same techniques, sample content normalization methods can be applied to generate expression estimates that are comparable between samples and controls. These methods include total count normalization (e.g., RPKM/FRKM used in RNA-Seq), quantile normalization (including median or upper quartile normalization) or other normalization methods (e.g., DESeq used for RNA-Seq). In the case of RNA-seq, expression estimates can also be normalized by locus length specified in models provided by the ENSEMBLE or RefSeq.

VI.A.v. Filtering of Expression Counts.

In some cases, REC data can be filtered to remove specific data that can lower the overall quality of the results. In some cases, RECs with values that fall below a specified quality threshold can be eliminated. In some cases this threshold can be an absolute number for a threshold, reflecting the degree of expression in the REC. For example in RNA-Seq, thresholds for elimination can be RECs with less than 2, 5, 10, 15. 20 or 25 reads. In other cases, RECs that have high variability, that have poor correlation with copy number or that map to multiple regions of the genome (i.e., from repetitive sequences within the genome) can be removed.

VI.A.vi. Identification of CNAs Using REC Data

A variety of approaches can be used for identifying CNAs using LREC and/or AREC data generated by RNA-Seq, hybridization- or PCR-based methods. In general, REC data from the sample can be compared to one or more references to assign copy number status to corresponding genomic regions. This process can involve several steps including: (1) preparation of input data, (2) comparison of REC data between sample and reference(s) to identify regions with abnormal expression, (3) combining of REC data into segments with similar relative expression profiles and (4) assignment of copy number to the segments. Each of these steps vary depending on factors that can include: (1) methods used to generate the REC data, (2) the type and quality of REC data and (3) the algorithm(s) used for comparing the sample to the reference(s) and assigning copy number. The number of loci or alleles evaluated per genomic region and the methods of detection can determine the resolution of this approach in detecting CNAs.

VI.A.vi.a. Regional Locus Expression-Based CNA Detection (RLECNAD)

For locus-based CNA identification, regional expression counts from one or more loci can be used. Any set of data that gives an accurate representation of the total expression from loci in the sample can be used. The total expression from a locus can include the expression from all alleles of the locus and all transcript isoforms produced by the locus. A variety of algorithms and statistical analyses can be used to identify genomic regions where loci from the sample are generally overexpressed or underexpressed relative to the reference(s). In some cases, algorithms can also estimate the copy number in the aberrantly expressed region based on the magnitude of the overall relative change in expression compared to the reference(s). REC data can be generated from RNA-Seq. Similar approaches can be used for hybridization-based and amplification-based REC data. In cases in which other methods of generating REC data are used, the algorithms can take into account different formats of data, different issues of signal to noise, sensitivity and technical biases.

### VI.A.vi.a.1. Format of Input Data.

The form and the fraction of sample REC data that can be analyzed by the copy number detection algorithm(s) can vary depending upon both the algorithms used and the goals of the analysis. In some cases, the REC data from the sample(s) and reference(s) can be directly used in the subsequent RLECNAD algorithm without any additional modification. In some cases, the REC data can be combined or divided into windows either defined by the user or determined through an optimization process. In some cases, the bins can be determined by an algorithm that divides the genome into bins of variable length adjusted such that the number of potential uniquely mapping reads in each bin can be normalized across the genome. In other cases, the bins can be defined by biological boundaries such as exons, loci or genes.

In other cases, the data can be converted into a format that reflects the relative differences between the embryo and the reference, data referred to as relative regional expression values (RREVs). Any value that qualitatively or quantitatively captures this comparison can be used. In some cases, the RREVs can be the absolute differences from the reference (i.e., sample REC−reference REC). In some cases, these RREVs can be used directly for subsequent analyses. In other cases, only absolute differences beyond certain thresholds can be used. The threshold for upregulation can be greater than a 1, 5, 10, 20, 25, 30, 35, 40, 50, 75, or 100% change. The threshold for down-regulation can be a 1, 5, 10, 15, 20, 25, 30, 35, 40, 45, 50, 55, 60, 65, 70, 75, 80, 85 or 90% change. Expression levels inside of the two threshold boundaries can be considered similar to the reference. The threshold can be set arbitrarily or based on empiric data or modeling.

In other cases, the RREVs can be fold-changes (i.e., sample REC divided by reference REC). In some cases, the fold-change data can be used directly for subsequent analyses. In other cases, threshold(s) can be applied to assign up- or down-regulation or no change. The threshold for upregulation can be a ratio greater than 1, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2, 2.25, 2.5 or 3. Threshold for down regulation can be less than 1, 0.95, 0.9, 0.85, 0.8, 0.75, 0.7, 0.65, 0.6, 0.55, 0.5, 0.45, 0.4, 0.35, 0.3, 0.25, 0.2, 0.15 or 0.1. Expression levels not outside of the upper and lower threshold values can be considered as no-change. In some cases, the thresholds can be determined by the user. In other cases, the thresholds can be based on optimal values determined using reference data. In some cases, the relative log ratios can be generated by taking the log 2 of the fold changes.

In other instances, a sign can be applied to a difference between the embryo and the reference. For example, RREVs based on absolute differences or ratios can be assigned a qualitative value of + for values above a threshold, − for values below a threshold and 0 for values in between the threshold. The threshold for upregulation can be set to a value that can be greater than 1, 2, 5, 10, 15, 20, 25, 30, 35, 40, 50, 60, 70, 80, 90,100, 125, 150, 175, 200, 225, 250, 275, or 300% of the reference value. The threshold for down-regulation can be set to be lower than 1, 2, 5, 10, 15, 20, 25, 30, 35, 40, 50, 60, 70, 80 or 90% of the reference value.

In some cases, thresholds for RREVs can be set based on standard deviations or other statistical measures of variance of the reference data. The upper threshold can be set at more than 0.1, 0.2, 0.3, 0.4, 0.5, 0.6, 0.7, 0.8, 0.9, 1, 1.5, 2, 2.5, 3, 3.5, 4, 4.5 or 5 standard deviations above the reference mean. The lower threshold can be set at below 0.1, 0.2, 0.3, 0.4, 0.5, 0.6, 0.7, 0.8, 0.9, 1, 1.5, 2, 2.5, 3, 3.5, 4, 4.5 or 5 standard deviations below the reference mean.

In cases in which an algorithm calls copy number based on the assumption that there is a positive correlation between copy number and expression level, it can also be possible to modify the expression data from loci that have an inverse correlation with copy number so that alterations in expression of these loci can be properly taken into account. In some cases, the relative changes can be corrected by taking the inverse of the change. In other cases, the response to different copy number states can be modeled for the gene and then converted to the appropriate median response for loci with a positive correlation. In some algorithms, there is no assumption about the correlation between copy number and expression level for loci and the algorithm can be trained with an appropriate dataset so that the responses of loci to different copy number states can be modeled.

### VI.A.vi.a.2. Extent of REC Data Used.

In some cases, all REC data generated from the sample(s) and reference(s) can be used. In other cases, only a subset of REC data can be analyzed. In some cases, only loci with particular biologic characteristics can be included for the purposes of improving the quality of input data such as high expression, high correlation with copy number or low biologic variability. In other cases, a subset of REC data can be used to restrict the analysis to particular genomic regions or to reduce the cost and/or time to analyze data. In these cases, loci from specific genomic regions can be selected. Loci can be selected to cover each chromosome at a particular density or at particular locations within the chromosome such as distance from the centromere and/or telomeres. Loci can also be selected to cover the genome or transcriptome at a certain density.

### VI.A.vi.a.3. References for RLECNAD

A variety of references can be used for evaluating the expression of loci in the sample. For the purposes of comparing the REC values from a sample to those of a reference, any reference that can facilitate inference of copy number in the test sample can be used. In some cases, an internal reference can be used from one or more regions of the genome in the sample, often referred to as reference-free analysis. In some cases, the internal reference can be the expression from a set of loci that have low variability in expression. In other cases, the internal reference can be from one or more entire chromosomes. In other cases, the internal reference can be from the entire transcriptome. In some cases, the internal reference can be the median expression of the region. In other cases, the internal reference can represent the mean expression of the region.

In other cases, REC data can be derived from other samples, e.g., human embryos or embryo biopsies generated under similar conditions and at a similar stage of development to the sample being evaluated. In other cases, the reference can be derived from REC data from more than 1, 5, 10, 50, 100, 1000, 5000, 10,000 embryos. In some cases, the reference can be derived from one or more embryos in which genotypic information is available pertaining to the genome copy number status for some or all of the loci that are evaluated. In other cases, the reference can be generated from one or more embryos in which there is no genotypic information available. In some cases, the embryo(s) comprising the reference can be matched to the sample based on biologic factors that might affect embryonic locus expression. Such factors include, but are not limited to (1) biologic conditions of one or both parents such as age, health status, genotype, diet, body habitus, history of illness or environmental exposure, (2) the specific assisted reproductive methods used to produce the embryo(s) such as ovarian stimulation protocol, method of gamete retrieval, technique of fertilization, embryo culture conditions and biopsy method and (3) the methods used to generate the transcriptome data. In some cases in which more than one embryo is used for generating the reference REC values, the reference REC values can represent the median value of the RECs in the reference set. In other cases, the reference REC can be derived from the means of values in the dataset. In other cases, the reference REC can be derived from statistical distributions fit to the expression values of each region in the dataset.

### VI.A.vi.a.4. Algorithms for RLECNAD

A variety of algorithms can be used to evaluate locus REC data to assign copy number status of corresponding genomic regions. Essentially, these algorithms can compare the REC data of the sample to the reference(s), segment the transcriptome into regions with similar relative expression and assign copy number to the segments. In some cases, an algorithm can be used that was originally developed for comparative genome hybridization array data. In some cases, an algorithm can be modified to apply to transcriptome data.

### VI.A.vi.a.4.(a) Assumptions of Distribution for RLECNAD

In some cases, segmentation algorithms can require assumptions about the distribution of the sample and reference data in order to identify differences between the sample and reference. For these purposes, the data can be assumed to be of a Poisson, Gaussian or negative binomial distribution or a mixture of distributions. In other cases, no underlying assumptions about the distribution of the data can be required.

### VI.A.vi.a.4.(b) Change-Point Algorithms.

A variety of algorithms that identity abrupt changes in relative expression across the transcriptome can be used. These abrupt changes can delineate the boundaries of the regions with altered copy number. In many of these algorithms, statistical analyses are incorporated to determine whether segments differ between the sample and reference. In some cases, circular binary segmentation can be used (Olshen et al (2004) Biostatistics 5: 557-72, incorporated herein by reference). CBS can be a recursive method in which the breakpoints can be determined on the basis of a test of hypothesis, with the null hypothesis of no difference in copy number. This method can minimize variance within segments and maximize variance between segments. In other cases, a piecewise constant regression model can be used in which parameters are estimated by maximizing a penalized or weighted likelihood or through the use of Bayesian statistics (Picard (2005) BMC Bioinformatics 6:27, Hupe (2004) Bioinformatics 10: 3413 and Rancoita (2012) BMC Bioinformatics 10: 10, each incorporated herein by reference). Segmentation can also be performed using Hidden Markov Models (HMM) to assign windows of the transcriptome into a fixed number of possible states via an emission distribution (can be Gaussian), and segment by combining consecutive windows with same states (Fridlyand et al (2004) J. Multivariate Analysis 90: 132-150 and Marioni (2006) Bioinformatics 22: 1144-46, each incorporated herein by reference). Under HMM, segmentation and classification can promote each other by allowing probabilistic parameters in the model to learn from data through algorithms like Expectation Maximization (EM). REC data can also be segmented by minimizing Bayesian information criterion (BIC) (Xi (2011 PNAS 108: E1128-36, incorporated herein by reference), least absolute shrinkage estimator regression methods (LASSO) (Boeva (2012) Bioinformaties 28: 423-25, incorporated herein by reference), regression tree (Chen (2012) Cancer Res 72: nr2487, incorporated herein by reference) mean-shift (Abyzov (2011) Genome Res 21: 974-84, incorporated herein by reference), total variation minimization (Nilsson (2008) Genuine Biology 9: R13, incorporated herein by reference), total variation least squares and probabilistic approaches (Carter (12) Nature Biotech 30: 413-21, incorporated herein by reference). In some cases, a combination of segmentation methods can be utilized.

### VI.A.vi.a.4.(c) Smoothing Methods

Algorithms that estimate copy number changes as continuous curves can also be employed. These methods can be referred to as smoothing methods. Smoothing methods that can be used include wavelet regression method with Haar wavelet (Hsu (2005) Biostatistics 6: 211, incorporated herein by reference), quantile smoothing regression (Eilers (2005) Bioinformatics 21: 1146-53, incorporated herein by reference) and a segmentation method based on a doubly heavy-tailed random-effect model (Huang (2007) Bioinformatics 23: 2463-9, incorporated herein by reference).

### VI.A.vi.a.4.(d) Statistical Testing Methods.

In some cases, REC data can be evaluated by a statistical hypothesis test at each window (Yoon (2009) Genome Res 19: 1586-92) or several consecutive windows (Xie (2009) BMC Bioinformatics 10: 80).

### VI.A.vi.a.4.(e) Segment Interpretation

In some cases, the segment(s)s of the transcriptome that are defined by one or more of the above algorithms as differing from the reference can require further interpretation to assign a copy number state for each segment. In some cases, the copy number state can be based on cutoffs of the relative expression counts. These cutoffs can be defined by the user, derived empirically, optimized for designated sensitivity and/or specificity or based on error modeling of the algorithm.

VI.A.vi.b. Regional Allele Expression-Based CNA Identification (RAECNAD)

In some cases, CNAs can be identified by analyzing the expression of alleles from transcribed loci. Expression of alleles of a locus can be distinguished by the presence of one or more informative polymorphisms that are present and detectable in the RNA. Polymorphisms that are informative can be ones in which different alleles of the polymorphism are present in the transcribed sequences of alleles of a locus, thereby allowing for transcripts from different alleles of the locus to be distinguished molecularly. Single nucleotide polymorphisms (SNPs) can be used for assessing allelic expression. SNPs can be biallelic, and each SNP can be used to track the relative expression of two different species of RNA. Any polymorphism that can distinguish alleles of a locus can be used to detect CNAs using allelic expression data.

Changes in copy number can change the number of alleles for loci affected by the CNA (see e.g., FIGS. 3 and 4). For deletions, an allele can be lost. For hemizygous loci (i.e. monoallelic loci), a deletion can result in the complete absence of the loci. For loci that are normally biallelic, a deletion can lead to the presence of only a single allele, a process known as loss of heterozygosity (LOH). LOH can also arise if there is a type of uniparental disomy (UPD) in which there are two copies of the same chromosomal homologue, essentially resulting in two copies of the same alleles for all loci on the chromosome.

A gain in copy number can lead to a gain in an allele. For a monoallelic locus, it can increase its copy number by 2-fold. For heterozygous biallelic loci, a gain can double the copy number of one allele while not affecting the other allele. For homozygous biallelic loci, a gain can result in a 50% increase in copy number. In situations such as meiosis I nondisjunction, a copy number gain can lead to the gain of an allele that differs from the other two, resulting in triallelism for some loci.

These alterations in copy number of alleles can also be reflected by changes in expression of the alleles for dosage sensitive loci. Deletions can be detected by identifying genomic regions on hemizygous chromosomes (i.e., some of the X and Y chromosomes in mammalian males) that lack sequences from the loci, including polymorphisms. Deletions in autosomal chromosomes can cause LOH. LOH due to deletions can be distinguished from those associated with UPD based on the level of expression of the allele: deletions can have half of the level of expression of the loci whereas UPD can have normal levels of expression from loci. Copy number gains of a genomic region can be identified through an increase in expression of alleles on the chromosomal region that has increased in copy number.

Different approaches can be used to detect CNAs depending upon the genotypic information available for the alleles. In some cases, there is no information available pertaining to which alleles of SNPs in a genomic region are linked (i.e., physically located on the same strand of DNA, also known as the same chromosomal homologue). In this case, SNP alleles can be considered to be unphased. In other cases, it can be possible to determine which SNPs alleles are associated with which chromosome, a situation in which the SNP genotypic information can be referred to as being phased. Phasing of haplotypes can be determined through analyzing genotypic information from the parents or relatives, gametes or haploid cells derived from the parents or from haplotype data from populations or unrelated individuals (e.g., Browning Browning and Browning (2011) Nature Reviews Genetics 12: 703-714, incorporated herein by reference). In some cases, the parental origin of haplotypes can be determined, meaning that it can be determined which chromosomal haplotypes originated from which parent. This special type of phasing can be referred to as parental linkage phasing. To determine parental linkage phase, genotypic information from the parents or other relatives can be used to infer inheritance of haplotypes. The phasing status of SNP alleles can impact the approach used to detect CNAs using allelic expression data (see e.g., FIG. 3).

Several different approaches can be used to detect CNAs using allelic expression data: haplotype expression-based, allelic expression ratio-based and LOH-based CNA detection (see e.g., FIGS. 3 and 4). The haplotype expression-based method can be similar to the locus expression-based method in that it can look for regional perturbations in the expression levels of haplotypes when compared to one or more reference(s) to identify CNAs. Differences between locus-based and haplotype-based approaches can include: (1) the haplotype expression-based approach can be limited to analysis of loci with informative polymorphisms, (2) the magnitude of a changes in expression in response to a CNA can be greater for alleles than loci and (3) when parental linkage is established, it can be possible to determine which parental chromosomal homologue is affected by a CNA. For this method, the two haplotypes can be evaluated independently and then the results can be combined to generate a copy number status for the test sample.

The allelic expression ratio-based method can identify CNAs based on imbalances in ratios of polymorphic alleles when compared to a reference. When there is a change in the copy number of an allele, it can change the relative abundance of the transcript and its distinguishing polymorphic alleles. For example, a copy number gain can change the ratio of allelic expression in a locus from 1:1 to 2:1 or 1:2. An imbalance in allelic ratios cannot necessarily identify which type of CNA has occurred in a genomic region since an imbalance could be caused by either a gain of one allele or loss of the other. In some cases, this approach can be combined with one of the other methods of CNA detection to determine which type of CNA can most likely be present. The allelic expression ratio method can be used with phased or unphased data. Phasing can improve the detection as the ratios can be formulated to compare the expression levels of one chromosome to those of the other.

Since the previously described allele-based approaches focus on informative polymorphisms, it can be beneficial to include an evaluation for loss of heterozygosity. A variety of methods can be used to look for the presence of unexpectedly large regions of homozygosity.

The approaches to analyzing allelic expression that can be used can be impacted by whether the polymorphism genotyping data are phased or unphased, and if phased, whether the parental linkage is established or not.

### VI.A.vi.b.1. Phased Regional Allelic Expression-Based CNA Detection (RAECNAD)

### VI.A.vi.b.1.(1) Parental Linkage Phased RAECNAD

In an embryo in which the haplotypes can be phased and parental origins of haplotypes can be defined, CNAs can be detected using either of the two previously described allelic expression approaches, evaluating haplotype expression or allele expression ratios. In some cases, analysis of haplotype expression can provide more specific information about the type of CNA and can determine which parental chromosome harbors the CNA. As mentioned previously, this method can be similar to the approach used for locus expression-based CNA detection, except that the analysis can involve the assessment of the expression of the 2 haplotypes. The sources of references can be any of those described previously for locus-based expression approaches. In the context of samples with parental linkage, the expression data from the 2 parental haplotypes can be compared to reference haplotype data of the respective gender (e.g., allelic expression from maternal chromosome 15 of the sample is compared to of maternal chromosome 15 allelic expression data in the reference(s)). By comparing to the expression data from the same gender parent, this method can take into account any differences in expression between parental alleles. There are some data indicating that there can be differences in expression of maternal and paternal alleles in preimplantation embryos. Any of the algorithms previously described for locus-based expression CNA detection can be used for analyzing these haplotype expression data. Once the expression data of the 2 haplotypes of the sample have been undergone CNA analysis, the two sets of results can be combined to generate a report of CNAs in the sample. Of note, this type of analysis can also determine which parental chromosomal homologue is affected by the CNA(s). Knowledge of the parental origin of the CNA can also be helpful in interpreting CNAs since different types of CNAs have different probabilities of arising in the male or female germline. For example, in some cases, most aneuploidies can arise maternally while most CNVs can arise paternally.

In some cases, the allelic expression data can be evaluated by looking at relative abundance of alleles of informative loci through use of an allelic expression ratio (AER). The AER can be expressed in a variety of formats: maternal: paternal, paternal:maternal, paternal fraction (paternal/(paternal+maternal)), maternal fraction (maternal/(maternal+paternal)), % Paternal (paternal/(maternal+paternal)×100) or % maternal (maternal/(maternal+paternal)). The AER of the sample can then be compared to similar AER data generated from one or more of the previously described references.

A variety of statistical analyses can be used to determine if allelic ratios of the sample differ significantly from those of the reference(s). In some cases, ratios can be transformed or processed prior to the comparison to reduce noise, account for biases introduced by the technique, correct for mosaicism or eliminate any other influences that do not pertain to allelic expression. In other cases, the AERs are not be transformed. In some cases, a binomial test can be performed to determine if the sample AER differs significantly from the reference AER. In some cases, the results can be corrected for multiple testing using FDR or similar correction. In some cases, error parameters for miscalling genotypes can be included as described by Nothnagel, et al. ((2011) Human Mutation 32: 98-106, incorporated herein by reference). In other cases, a Bayesian model developed by Skelly et al (Skelly, et al. (2011) Genome Res 21: 1728-1737, incorporated herein by reference) can be used in place of the binomial test to identify allelic imbalance. In cases in which statistical analyses are performed, AERs from the embryo can be considered to differ from the reference AER if the p value is less than 0.1, 0.05, 0.01, 1E−2, 1E−3, 1E−4, 1E−5, 1E−6, 1E−7, 1E−8 or 1E−9. In some cases, a difference of more than 1, 2, 5, 10, 15, 20, 25, 30, 35, 40, 50, 60, 70, 80, 90,100, 125, 150, 175, 200, 225, 250, 275, or 300% can be considered to indicate that the embryo AER differs from the reference AER. In some cases, statistical analyses can be performed on more than one AER to improve accuracy due to the noise of the system.

Following individual analyses of AERs, some or all of the data can be combined to identify contiguous regions that differ significantly between the embryo and the reference. In one approach, a defined window of a certain number of SNPs can be chosen to identify allelic bias. In other cases, groups of AERs can be analyzed by approaches such as (1) simple smoothing: the log of the AER for a SNP can be determined by averaging the log AER for the SNP and a defined number of neighboring SNPs, (2) Z-score approach: assigning Z scores for the AERs for each SNP and then determining Z scores of windows of consecutive SNPs, (3) ergodic hidden Markov model (HMM): models genomic state based on HMM states of total expression and allelic ratios of the sample and (4) left-to-right HMM: models genomic state based on models from expression and AERs from all samples. These HMMs also can take into account that AERs can be expected to be consistent across a transcript (see e.g., Wagner, et al. (2010) Plos Computational Biology 6: e1000849, incorporated herein by reference).

Since these allele expression-based approaches use informative (heterozygous) SNPs, they are not suited for detection of loss of heterozygosity, which can be marked by a stretch of homozygous polymorphisms. A variety of approaches can be used to detect abnormally long stretches of homozygous SNPs, which can be consistent with LOH. In some cases, a hidden Markov model can be used in which SNP interniarker distances, SNP-specific heterozygosity rates, and genotyping error rate are incorporated such as that described by Beroukhim et al ((2006) PLOS Comp Biol 2: e41, incorporated herein by reference). As mentioned previously, the detection of LOH can indicate a deletion of a disomic region or the presence of uniparental disomy. These 2 possibilities can be distinguished through the analysis of the region of LOH with the locus expression-based approach.

### VI.A.vi.b.1.(2) Phased RAECNAD

In some cases, embryos are haplotyped but the parental origins of haplotypes are not defined. The three approaches previously described for expression data with parental linkage haplotype information can also be used for phased expression data in which parental linkage is not established with a few allowances taken for the reduced information.

For the haplotype expression based approach, the expression profiles of the 2 haplotypes can be compared to haplotype expression data from the reference(s) that also lack parental linkage information. Reference sources can be the same as described above. The same algorithms can be used as described above for data with parental linkage information.

For allelic expression ratio-based analyses, the ratio of expression can correspond to haplotypes without reference to parental origin. AERs can include: haplotype1:haplotype2, haplotype 2: haplotype1, haplotype 1 fraction (haplotype 1/(haplotype 1+haplotype 2)), haplotype 2 fraction (haplotype 2/haplotype 2+haplotype 1), haplotype 1% (haplotype 1/(haplotype 1+haplotype 2)×100) or haplotype 2% (haplotype 2/(haplotype 1+haplotype) 2×100). Comparisons of sample AER data to identically formatted AER data from references can be performed as described above for the parental linkage phased data.

### VI.A.vi.b.2. Unphased RAECNAD

In other cases, allelic expression data from a sample can be analyzed without the benefit of haplotype information. In this scenario, allelic expression ratios (AER) can be used to identify abnormalities of allelic expression. In some cases, the AER can be the ratio of the expression level of the higher expressed allele divided by the expression level of the lower expressed allele. Since it is not known which alleles are co-localized to a chromosome in the case of samples without haplotype information, regions in which the AERs are skewed significantly from the reference can be identified. The reference can be any of those described above for evaluating AER in haplotype phased samples. The analysis can be the same as used for phased allele ratios in which regional differences in allele ratios are identified. One difference as compared to phased data is that it cannot be determined which chromosomal homologue has relative increased expression.

### VI.B. Breakpoint Identification-Based CNA Detection (BICNAD)

In some cases, the evidence of genomic abnormalities such as deletions or duplications can be identified due to the recognition of the breakpoint in the sequence. In some cases, the breakpoint(s) can be identified by the recognition of a breakpoint sequence, i.e., a sequence joining two sequences that are not normally joined (i.e., not joined in the genome and not joined by alternative or transplicing). Breakpoint sequences can be identified in RNA-Seq data through the presence of a ‘split read,’ a read in which segments of the read align to different regions of the genome. These split reads can then be filtered to remove reads that could be explained by RNA processing.

In the case of paired end sequencing (i.e., sequencing of both ends of library clones in RNA-Seq), breakpoints can also be detected when the paired reads flank but do not span a breakpoint. In this scenario, the breakpoint can be identified as a result of the paired sequences not aligning to the expected region of the genome when the estimated size of the intervening sequence between the ends of the clone and allowances for splicing are taken into account. There are a variety of algorithms that can flag such discordant paired ends.

In some cases, the reads can be extended through the residual sequence extension approach as described by Liu et al ((2013) BMC Bioinformatics 14: 193, incorporated herein by reference). In some cases, the results can be filtered based on read number, sequence similarity, read position distribution.

A number of algorithms have been developed for identifying chimeric transcripts using single read and/or paired read RNA-Seq data including ChimeraScan, defuse, FusionFinder, FusionHunter, FusionMap, MapSplice, ShortFuse, TopHat-Fusion, FusionSeq and FusionQ (Carrara (2013) BMC Bioinformatics 14: S2 and Liu (2013) BMC Bioinformatics 14: 193 incorporated herein by reference).

### VI.C. Expression Signature-Based CNA Detection (ESCNAD)

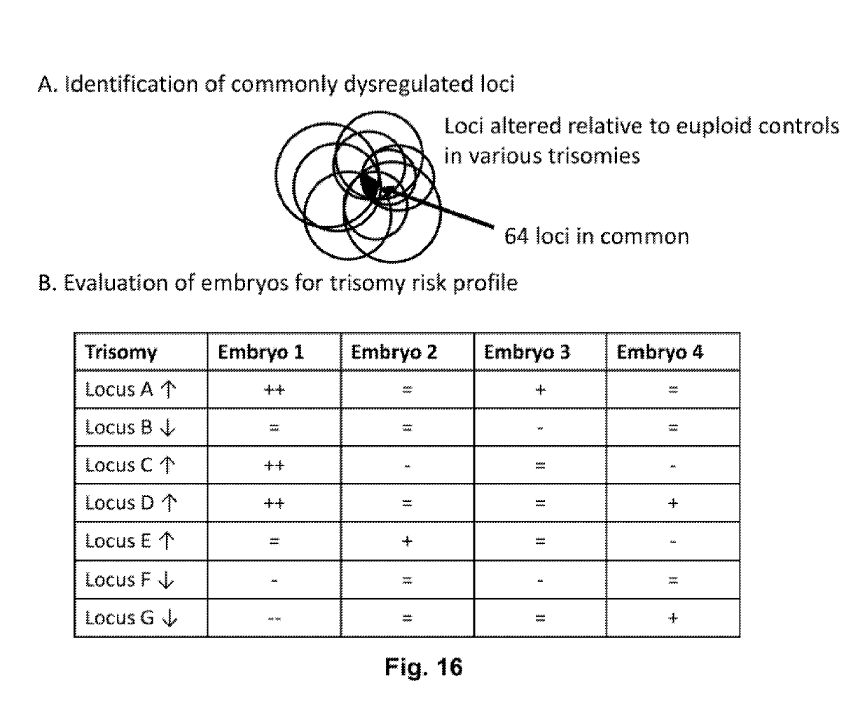

In some cases, the presence of CNAs can be determined through the identification of expression profiles that are associated with genomic copy number alterations (see e.g., FIG. 16). In some cases, this approach can look for expression profiles or signatures without any reference to the genome. In some case, this approach can incorporate not only primary alterations but also those expression alterations that occur in response to the primary alteration. These responses can be secondary or more complex responses to one or more dosage-mediated alterations that arise from one or more CNAs. In some cases, by comparing expression profiles of different CNAs, expression signatures associated with classes of CNAs can be identified. In some cases, some CNAs can have common effects on the transcriptome. For example, Sheltzer et al ((2012) PNAS 109: 12644-9, incorporated herein by reference) found that gains of chromosomes in a variety of species can lead to upregulation of expression of loci associated with responding to generalized cellular stress.

VI.C.1. Identifying Expression Profiles Associated with CNAs

In some cases, the first step of this approach can be to identify expression alterations associated with CNAs. To achieve this goal, locus expression profiles of one or more samples from embryos with one or more CNAs can be compared to one or more references to identify alterations in the expression of loci associated with the CNA(s). Expression data can be generated using any of the sequence-, hybridization- or amplification-based methods described herein. In some cases, the presence or absence of CNAs in the test and reference samples can be determined by genome analysis. In some cases, the CNAs can be defined by the method of expression-based and breakpoint identification-based CNA detection methods as described herein. In some cases, the expression data from the test sample can be produced from a single embryo. In other cases, the test sample can be produced from more than one embryo. In some cases, more than one test with the same CNA can be included to aid in identifying loci that are considered altered in expression. In some cases, the reference can be composed of expression data from one or more embryos that have been shown to carry no detectable CNAs. In other cases, the reference can be composed of data from embryos that carry one or more different CNAs that are not present in the test sample. In some cases, a differential expression algorithm can be used to identify loci that are statistically significantly altered in expression relative to the reference. Examples of differential expression programs for RNA-Seq include, but are not limited to Cuffdiff, edgeR, DESeq, PoissonSeq, baySeq and limma (Rapaport (2013) Genome Biology 16: R95, incorporated herein by reference). Examples of differential expression program for microarray data include, but are not limited to, SAM, CyberT, RankProd and ANOVA-SCA (Cordero (2008) Brief Funct. Genomic Proteomic 6: 265-81). In some cases, empirically derived thresholds for relative expression can be used identify expression alterations. In some cases, the fold-change threshold can be set at more than 1.5, 2, 2.5, 3, 4, or 5-fold increase. In other cases, the fold-change threshold can be set at less than 0.75, 0.5, 0.4.0.3.0.2 fold change. In some cases, loci localized to the region harboring the CNA can be filtered out to eliminate primary effects.

Loci identified as being differentially expressed or altered in expression as a result of one or more CNAs can be further analyzed to identify commonly altered loci or pathways in response to a particular CNA or class of CNAs. A variety of enrichment analyses can be used to identify loci and/or and biological pathways that are commonly altered in expression in association with a CNA or class of CNAs. One approach uses tests of proportion to determine whether a significant fraction of the loci in an expression profile are among those that are identified as differentially expressed in a dataset (e.g., analytic tools in Database for Annotation, Visualization and Integrated Discovery (DAVID); see Dennis et al (2003) Genome Biology 4: 3 and Huang et al Nature Protocols 4: 44-57, each incorporated herein by reference). A second approach uses tests of distribution to determine whether the members of an expression set are overrepresented at either extreme of the list of all loci ranked by their degree of differential expression (e.g., gene set enrichment analysis (GSEA), see Subramanian et al (2005) Proc. Nat. Acad. Sci 102: 15545, incorporated herein by reference). Another strategy to identify patterns of commonly altered expression can involve using tests of proportion or distribution to determine whether any loci are coordinately differentially expressed (e.g., CMap, see Lamb (2006) Science 313: 1929, incorporated herein by reference).

### VI.C.2. Developing a Scoring System for a CNA Profile.

Once expression profiles associated with one or more types of CNAs have been identified, an expression profile for a sample can be evaluated to determine how similar its pattern of expression is to any of the signatures of CNAs. A variety of scoring systems can be developed to reflect the degree of similarity to the CNA-associated profile(s). In some cases, a score can be produced using the expression levels. In one example, the expression values of loci that are relatively upregulated in the profile are summed along with the negative expression values for loci that are downregulated in the profile. In other cases, relative expression values of the sample can be used to generate a score. In one example, a score can be generated by adding the relative expression values for the sample, taking the straight fold change value for relative increase the profile and adding the inverse for those that show relatively decreased expression in the profile. In some cases, the expression values for loci in the profile can be weighted based on the degree of correlation with a CNA or class of CNAs or average or median fold change for the locus. Thresholds for scores can be determined empirically taking into account sensitivity and specificity as well as positive and negative predictive power of thresholds.

### VII. FILTERING OF DETECTED CNAS

In some cases, the list of CNAs generated from one or more of the above approaches for CNA detection can be further processed to remove false positive results and prioritize among identified CNAs. In some cases, the CNA detection results can be filtered based on the CNA length, confidence score or presence in the embryo dataset of other clinical datasets. In some methods for identifying CNAs, a p value and/or confidence interval can be supplied for each CNA. These values can be supplied with the results to express the probability of the finding. In some cases these p values can be corrected for multiple testing. In other cases, a CNA can be reported as simply being present or not based on a cut-off for p values, corrected or uncorrected, such that p values above 1E−9, 1E−8, 1E−6, 1E−5, 1E−4, 1E−3, 1E−2 or 1E−1, are not considered present. In other cases, user defined criteria for selecting CNAs can be used. In other cases, other clinical data such as data embryo development, morphology and metabolism can be incorporated to modify the probability of the finding of a false positive or negative result. In other cases, the positive and negative predictive values of these analyses can be derived from clinical studies in which confirmatory genome analyses are performed in conjunction with this test. In some cases, CNA analysis can identify too large of a number of CNAs, which can indicate poor quality of the sample. In some cases, a certain number of CNAs or portion of the transcriptome can be used as a criterion for sample quality. In some cases, samples with less than 90, 80, 70, 60 or 50% of the transcriptome or genome estimated as being present can signify poor sample quality.

### VIII. INTERPRETING CNAs

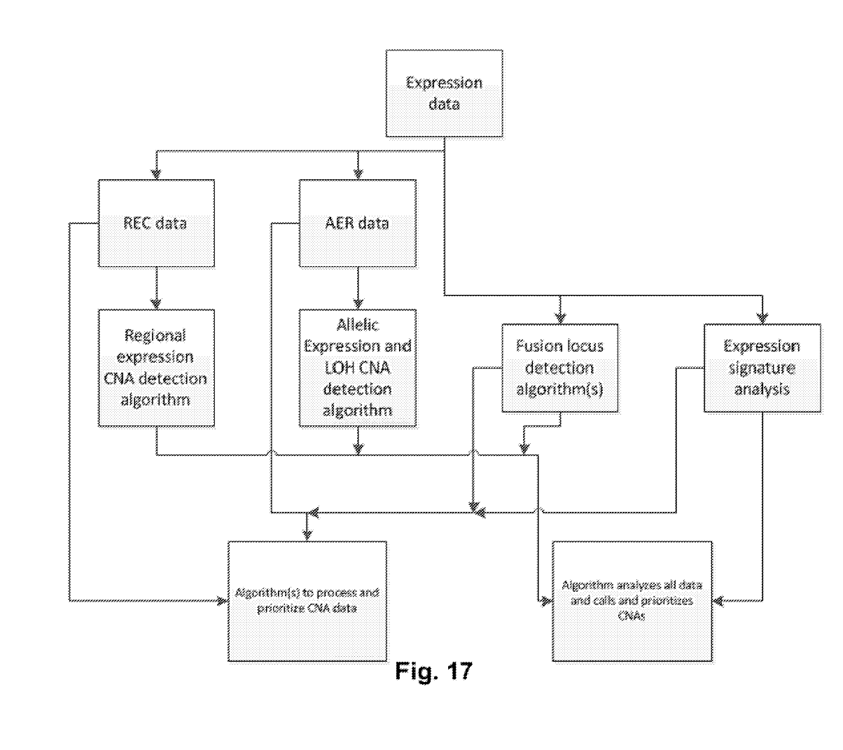

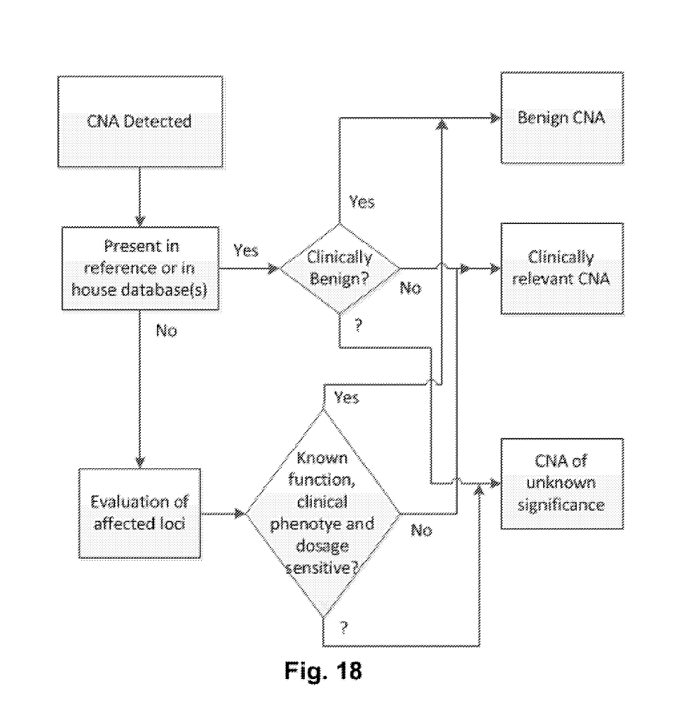

The relevance of a genomic abnormality (e.g., CNA) can be assessed to determine if it is pathogenic or benign (see e.g., FIG. 17). To determine the impact, databases that catalog genomic variants such as ENSEMBL (http://www.ensembl.org), the database of chromosomal imbalance and phenotype in humans using ENSEMBL resources (DECIPHER, http://www.sanger.ac.uk/PostGenomics/decipher/), the database of genomic variants (DGV http://projects.tcag.ca/variation) and the variant effect predictor (http://www.ensembl.org/info/docs/tools/vep/index.html) can be consulted to determine the likelihood that a particular CNA will have phenotypic or health consequences. Other factors that can be considered in assessing the biological impact of a CNA include the size of the CNA, genomic content and evidence of dosage sensitive loci in the online Mendelian inheritance in man (OMIM) database (www.ncbi.nlm.nih.gov/omim). The variant effect predictor also can provide insight into the potential effects of variants using sequence ontology, overlap with known regulatory features, location relative to high information parts of transcription factor binding sites. Review of current literature can also provide insight. In some cases, genomic analysis can be performed on the parents to determine if either possesses the observed abnormality. Based on some or all of these analyses, an estimation of the likelihood of the pathogenicity of a CNA can be determined.

Another approach for interpreting the biologic effects of CNAs relates to assessing the secondary alterations in transcriptome data (i.e., alterations that are not directly related to the change in copy number such as alterations in the expression of loci from unaffected genomic regions). The identification of secondary responses in samples can provide indicate potential biologic effects of the CNA and, as mentioned before, support for the existence of a CNA.

### IX. CNA DETECTION METHODS CAN IDENTIFY A NUMBER OF ABNORMALITIES IN THE EARLY EMBRYO

The presented expression-based detection methods in concert with the other methods can detect aneuploidies. Large segmental aneusomies, gains or losses of segments of chromosomes, can also be identified. The lower limits of the size of CNAs that can be detected by these approaches can vary, depending on a number of factors that include, but are not limited to, the stage at which the embryo is sampled, the size of the sample, the method used to evaluate the transcriptome, the depth and breadth of the coverage of the analysis of the transcriptome and the analytic algorithms used to detect CNAs. It is also likely that this method can detect alterations in ploidy based on disproportionate transcriptional response of select loci to this condition. The ability to detect large CNAs is of great clinical relevance because of the high prevalence of large CNAs in human preimplantation embryos.

Early embryos can also have a high frequency of genetic mosaicism. Mosaicism can be a condition in which one or more genetic alterations are present in only a subset of cells. One mechanism for mosaicism is the development of the genetic alterations in a cell of the embryo after the first mitotic division. This can also be the case for genetic alterations detected by transcriptome analysis in early embryos. Mosaicism can be detected using locus and allele expression-based approaches in which the results are intermediate relative to standard copy number states.

### X. APPLICATIONS

### X.A. Detection of Chromosomal Abnormalities

The compositions and methods of this disclosure can be directed toward detection of CNAs. One class of CNAs in early human embryos is aneuploidy, which can involve gains or losses of chromosomes that do not result in a multiple of the haploid complement of chromosomes. Some of these aneuploidies can be lost in the early prenatal period. Approximately half of spontaneous abortions can be aneuploid, making this genetic condition the leading known cause of miscarriage. Aneuploidies can be present in about 4% of stillbirths and 0.4% of liveborns. A small subset of aneuploidies can be compatible with livebirth, mainly consisting of trisomies 13, 21 and 18 and the sex chromosomal abnormalities XO, XXY and XYY.

There are a number of clinical benefits to detecting chromosomal abnormalities in embryos prior to establishing a pregnancy. First, such genetic screening can improve outcomes of assisted reproductive technologies. The detection of aneuploidy, thereby preventing the transfer of aneuploid embryos to the female reproductive tract, can also improve the pregnancy rates. Second, this screening can help to lower the rate of multifetal pregnancies produced by ART. In the US, almost 30% of ART pregnancies are multifetal, mainly a result of more than one embryo being transferred in ART cycles. One of the rationales underlying the transfer of more than one embryo is to account for the possibility of aneuploid embryo(s) being transferred. Multifetal pregnancies can be associated with increased risks of numerous medical complications to the mother, fetus and newborn. By screening embryos for aneuploidy, a lower number of embryos, preferably a single embryo, can be transferred during an ART cycle, thereby reducing the risk of multifetal pregnancies while maintaining or even improving the chance that the cycle produces a liveborn child. Third, screening for chromosomal abnormalities can reduce the risks for having liveborn children with aneuploidy.

### X.B. Early Detection of Segmental Aneusomies

The compositions and methods of the disclosure can also be used to detect CNAs that affect a portion of a chromosome, which can be referred to as a segmental aneusomy. These genomic abnormalities can involve large regions of chromosomes, particularly toward the ends of chromosomes. A wide array of smaller genomic imbalances can be relatively common and can cause debilitating conditions. Examples of such genomic disorders include: a 3 Mb deletion of 22q11.2 that causes DiGeorge and velocardiofacial syndromes, a 5 Mb deletion of 15q11 that causes Angelman or Prader Willi syndrome depending upon parent of origin, a 1.5 Mb deletion of 17p that causes Charcot-Marie-Tooth syndrome, a 1.5 Mb duplication of 17p that causes hereditary neuropathy and liability to pressure palsies, and a 1.5 Mb deletion of 7q11 that causes Williams syndrome. Given that most of these deletions can impact the copy number of more than 20 loci, some are likely to be able to be detected with the previously described RNA-based methods.

### X.C. Early Detection of Uniparental Disomies

Uniparental disomy (UPD) can occur when there are 2 copies of a chromosome present, and both chromosomal homologues are from the same parent. In cases in which both homologues are identical, it is referred to as isodisomy. In cases in which the chromosomes differ, representing the two different homologues present in one parent, it is referred to as heterodisomy. Uniparental disomy can arise due to errors in the meiotic and early embryonic mitotic divisions, e.g., due to rescue of a trisomy or monosomy. In trisomy rescue, a trisomic zygote can subsequently lose the single chromosome from one parent, leaving two homologues from the same parent. In monosomy rescue, the sole homologue can be duplicated. UPD can have effects on any chromosome that is subject to genomic imprinting. Genomic imprinting can be defined as the differential expression of loci depending upon from which parent the chromosome was inherited. Five chromosomes have been defined as being imprinted based on clinical phenotypes and basic research: chromosomes 6, 7, 11, 14 and 15. Maternal UPD 6 can be associated with transient neonatal diabetes. Maternal UPD 7 can be linked to Silver-Russell syndrome. Full UPD for chromosome 11 can be lethal, but segmental paternal isodisomic UPD (iUPD) can be associated with Beckwith-Wiedemann syndrome. Maternal and paternal UPD 14 can be associated with a number of phenotypic and developmental abnormalities. UPD15 is one of the more common UPDs. Maternal UPD 15 can result in Angelman syndrome and paternal UPD15 can cause Prader Willi syndrome. By using methods described herein that can evaluate allelic expression in the transcriptome, UPDs can be identified. In the case of iUPD, loss of heterozygosity for the affected chromosomal region can be detected. For hUPDs, genotypic information from the parents can be used to determine that both chromosomal homologues in the embryo were inherited from one parent. The identification of UPD at this early stage can prevent the establishment of pregnancies with this class of disorders, many of which have phenotypic features that can impact health and well-being.

### X.D. Detection of Other Genetic Alterations in Concert with RCNAD

The data generated from analysis described herein can be used alone or in parallel with other genetic diagnostic approaches to detect a variety of other types of genetic alterations, directly or indirectly. Any alteration that is transcribed into a stable transcript in the preimplantation embryo can be amenable to direct mutational detection. These alterations can be associated with disease, disease susceptibilities or traits as mentioned, e.g., in Section I. A trait can be any specific characteristic of an organism that can be influenced by its genetics. Examples of traits include genetic diseases (both Mendelian and complex), gender, histocompatibility, susceptibility to disease, height, eye color, intelligence and athletic ability.

One example of how a trait can be identified in the early embryo is the determination of the sex of the embryo. The sex of the embryo can be determined through the evaluation of expression of X- and Y-linked loci. For example, an embryo that expresses loci on the Y-chromosome outside of the pseudoautosomal region and expresses X-linked loci at a level consistent with a single copy can indicate that the embryo is male. The absence of Y-linked expression and X-linked expression consistent with the presence of 2 X chromosomes (both X chromosomes are active in human preimplantation embryos) can indicate female gender. Determination of the sex of an embryo can be used to prevent the establishment of pregnancies with X-linked disorders and/or for family balancing.

In some cases, transcriptome profiling of cellular total RNA can be used to evaluate the mitochondrial genome. Genetic alterations that are transcribed from the mitochondrial genome can also be detected using the approaches for transcriptome profiling described herein. Furthermore, since there are thousands of copies of the mitochondrial genome per cell, analyses of the mitochondrial transcriptome can also be used to assess the number of mitochondria per cell.

In some cases, one or more genetic alterations of interest cannot be directly detected by RNA-based analyses. Loci that are not expressed in preimplantation embryos cannot be identified directly. Loci that are expressed at low levels can or cannot be detected directly depending upon the sensitivity of the methods used. In some cases, genetic alterations that cannot be detected directly can be detected indirectly by one of several methods. In some cases, the inheritance of a genetic alteration such as one or more mutations carried by one or both parents can be determined through linkage analysis. Linkage analysis can allow for the inheritance of genomic regions from the parents to be followed through the inheritance of closely linked polymorphisms. For example, whether an embryo inherited a mutation that causes Huntington disease from a parent can be determined. Huntington disease is an autosomal dominant disorder that can be caused by the abnormal expansion of a triplet repeat contained within the HTT (HD) gene. By using informative polymorphisms that are closely linked to this mutation, it can be determined whether a mutant or normal allele of this gene from the affected parent has been inherited.

A second indirect method for identifying inheritance of a mutation can be to identify an associated haplotype. In this approach, the inheritance of a mutation can be assessed through the determination of whether the embryo contains a haplotype that has been shown to be linked to the mutation. This approach can be used to detect a mutation that recently arose in a small, isolated population. One such example is a 3398delAAAAG mutation in breast cancer BRCA 2 gene, which can be linked to one of two rare haplotypes in French Canadians.

A third approach to identifying a risk for presence of a genetic alteration can be through the identification of primary or secondary alterations in the transcriptome. A mutation, although not transcribed, can impact the expression of one or more loci expressed in the embryo. A mutation can have a primary effect on one or more transcripts by affecting their transcription, processing or stability. One example of a mutation that can impact transcription is a mutation that alters the function of an imprinting control region causing a loss of expression of a locus from the appropriate parental allele. A mutation can also exert a secondary effect by impacting the transcription, processing or stability of a number of loci.

### X.E. Genetic Fingerprinting in Combination with RCNAD.

In some cases, genetic information that accompanies the RNA-based CNA detection method or that can be produced from additional genetic testing can be used to identify a group of alleles of polymorphisms that can serve to identify the embryo, often referred to as genetic fingerprinting. Depending upon the number of polymorphisms tested and the frequencies of alleles of these polymorphisms within the population, it can be possible to distinguish a genotype of an embryo from genotypes of other embryos, fetuses or people. Likewise, genetic fingerprinting information can be used to evaluate the relatedness of an embryo to other embryos, fetuses or people. Genetic fingerprinting data from the embryo could be useful for a number of applications. First, it could be used to identify the embryo. In the event that there was a question about the identity of an embryo that had previously undergone genetic fingerprinting, it would be possible to rebiopsy the embryo, perform RNA- or DNA-based genetic fingerprinting and determine if the embryo is the same as the one that was previously fingerprinted. Similarly, genetic fingerprinting could be used to determine if a fetus or child developed from a particular embryo. This type of follow up testing would be particularly valuable in the context of when more than 1 embryo is transferred and there is some benefit to knowing which of the embryos produced a fetus or child. Genetic fingerprinting can also be used to confirm that an embryo was produced by a given set of parents. Such testing can also be helpful in determining whether an embryo is the product of a set of collected gametes or a particular ART cycle. Genetic fingerprinting can also be used to detect contamination from exogeneous nucleic acids. Since the methods used for these types of analyses can be sensitive, the introduction of even small amounts of exogenous nucleic acids, particularly RNA or DNA, can potentially affect the results of these analyses. By performing genetic fingerprinting on the sample material and comparing these results to parental genetic fingerprinting data, it can be possible to identify contaminated samples through inconsistencies in the fingerprinting data such as the presence of alleles that are not carried by either parent.

### X.F. Assessment of Embryo Health and Developmental Potential in Concert with RCNAD

A transcriptome can provide information about the health and biological functioning of the embryo. By surveying transcripts associated with various biologic pathways, a variety of perturbations that can indicate compromised development, health and/or developmental potential can be identified. Abnormalities in the expression of loci that constitute the developmental signature of the stage at which the embryo was biopsied can reveal that the embryo has not developed properly. Examples of such genes in a blastocyst biopsy sample are the expression of loci involved in specification of the trophectoderm and preparation for implantation as well as imprinted loci that are reprogrammed during this period of development. Abnormalities in other classes of loci that are vital to cellular function, such as those involved in cell division, energy metabolism, biosynthesis, nucleic acid synthesis and repair, stress response, cellular signaling and programmed cell death can indicate compromised state of health. In some cases, the compromised health is due to genetic abnormalities present in the embryo. In some cases, the compromised health is due to current or past exposure to adverse environmental factors such as exposure to toxins or other insulting agents, infection or a suboptimal culture environment. The identification of a particular environmental insult can provide the opportunity for intervention that avoids or minimizes exposure or mitigates the consequences of exposure. This type of monitoring can be useful for assisted reproduction clinics in optimizing approaches to generating, culturing, manipulating and cryopreserving gametes and embryos. In some cases, the compromised health of an embryo can be due to a combination of genetic and environmental factors. In some cases, transcriptome profiles associated with high developmental potential can be identified through the analysis of transcriptome data from one or more embryos that have developed into healthy offspring. With recognition of a transcriptome profile of high developmental potential, the developmental potential of embryos can be assessed by the degree of similarity to this profile. In some cases, embryos classified as having high developmental potential can be selected for transfer.

### X.G. Evaluation of Mitochondrial Locus Expression Along with RCNAD

In some cases, a mitochondrial transcriptome in an embryonic sample can be analyzed in concert with RNA-based CNA detection. The human mitochondrial genome normally encodes 13 proteins, 22 transfer RNAs and 2 ribosomal RNAs. In one application, global expression of the mitochondrial transcriptome can be used to evaluate the number of copies present in embryonic cells. The number of mitochondria in human oocytes can vary over more than an order of magnitude. There are also data showing that oocytes that fail to fertilize can have lower numbers of mitochondria as compared to those that can be fertilized. Quantitation of mitochondrial cellular content can be a biomarker of developmental competence. Preimplantation mammalian embryos can become more metabolically active during the course of the preimplantation period. In some cases, a range of metabolic activity can correlate with a good developmental outcome. In some cases, expression of the proteins involved in energy metabolism can serve as a marker of health and developmental potential. In some cases, one or more mutations in a mitochondrial genome that cause human disease can be present in transcripts. In some cases, these mutations can be directly detected in a transcriptome.

### X.H. Combination of CNA Detection with other Diagnostic Approaches

In some cases, RNA-based CNA detection of the embryo can be combined with other genetic diagnostic approaches for the preimplantation embryo. In some cases, the additional analysis can be a direct evaluation of one or more genomic regions. Performance of both RNA- and DNA-based analyses can provide the benefit of allowing the results from one method to be validated or contested by the other. Genome analysis can also supplement transcriptome analysis by expanding the spectrum of genetic alterations that can be directly detected. In some cases, an additional biopsy sample can be used for proteomic analysis to evaluate a profile of proteins expressed in an embryo. RNA-based CNA detection analysis can be combined with a variety of other methods to assess embryonic health and competence. In some cases, the methods comprise evaluating the developmental progression of the embryo through time lapse imaging and assessing metabolism and secreted protein profiles through analysis of the embryo's culture medium.

### X.I. Storage and Dissemination of Embryo Genotypic Information

RNA-based CNA detection with or without additional genetic testing can generate millions of bits of information pertaining to the health and genetics of an embryo. Furthermore, some information from this analysis can indirectly provide genetic information pertaining to the individual(s) from which the embryo was generated. The massive amount of raw and processed data generated from this analysis can be stored in any manner that allows for archiving and retrieval, e.g., through memory storage devices accessed by computer. RCNAD with or without additional genetic testing can be applied to embryos from a number of species including human embryos. In some cases, there are rules and regulations that can govern the use and storage of these data. For clinical testing of human embryos, appropriate consents can be obtained from parties involved in producing the embryo and standard regulations can govern how these data and derivative summaries and reports are stored and disseminated. This information can be protected from access by any unauthorized individual. In some cases, the information can only be communicated to the ordering physician or his/her designee in accordance with state and federal laws. In some cases, an ordering physician can share this information with patients and medical staff who are directly involved in the clinical case. For analyses of nonhuman species and research applications, a variety of federal and state laws and regulations, policies of funding agencies and institutional rules and regulations can impact how RCNAD data are stored and disseminated.

In some cases, RCNAD screening of human embryos can be performed as a clinical diagnostic test. After information about specific genetic alterations is reported to the ordering physician, a medical professional can take one or more actions that can impact the assisted reproductive treatment plan or the testing or interventions performed on the embryo or the ensuing fetus, child or adult. In some cases, the findings can provide actionable genetic information to the patient or patients from whom the embryo was generated. For example, a medical professional can record information in the parents' medical record regarding the embryo's risk of having a CNA that can be associated with prenatal loss or postnatal disability and/or mortality. In some cases, this information can prevent the use of this embryo to establish a pregnancy. In other circumstances, this information can provide evidence for risks for disease or disability at later stages of development that warrant subsequent medical tests and interventions should the embryo be transferred and lead to establishment of a pregnancy. In some embodiments, a medical professional can provide a copy of these test results to other medical specialists.

In other cases, this testing can be performed for nonclinical purposes. In some cases, this testing can be used for research applications on human embryos to advance research pertaining to the understanding of embryo genetics and biology and improving methods to generate and evaluate embryos. In other cases, these analyses can be used for diagnostic purposes on nonhuman embryos. In some cases, this testing can be used for similar purposes of screening for CNAs in preimplantation embryos of other mammals, including many domestic species. In other cases, this testing can be used to advance biomedical research. In these applications, the scientists and staff directly involved in the experiments can have access to the information. For human embryo research, the data can be de-identified. In some cases, results from these analyses can be presented to other scientists or the lay community in the form of publications and/or presentations.

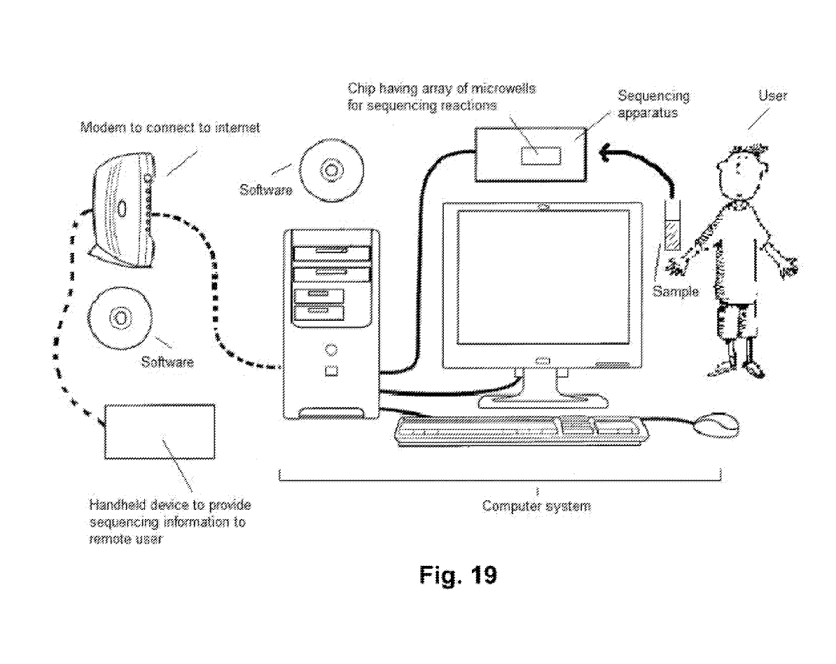

Any appropriate method can be used to communicate information pertaining to these analyses to another person. For example, information can be given directly or indirectly to a professional, and a laboratory staff member can input the report of embryo's genetic alteration into a computer-based record. In some cases, information can be communicated by making a physical alteration to medical or research records. For example, a medical professional can make a permanent notation or flag a medical record for communicating the risk assessment to other medical professionals reviewing the record. In addition, any type of appropriate communication can be used to communicate the risk assessment information. For example, mail, e-mail, telephone, and face-to-face interactions can be used. The information also can be communicated to a professional by making that information electronically available to the professional. For example, the information can be communicated to a professional by placing the information on a computer database such that the professional can access the information. In some cases, the information can be communicated to a hospital, clinic, or research facility serving as an agent for the professional. An exemplary diagram of computer based communication is shown in FIG. 19.

### XI. EXAMPLES

### XI.A. Example 1

### Demonstration of a High Correlation Between Copy Number and Locus Expression in Preimplantation Embryos

In this example, the effects of aneuploidy on the transcriptome of preimplantation mouse embryos were evaluated.

### Methods

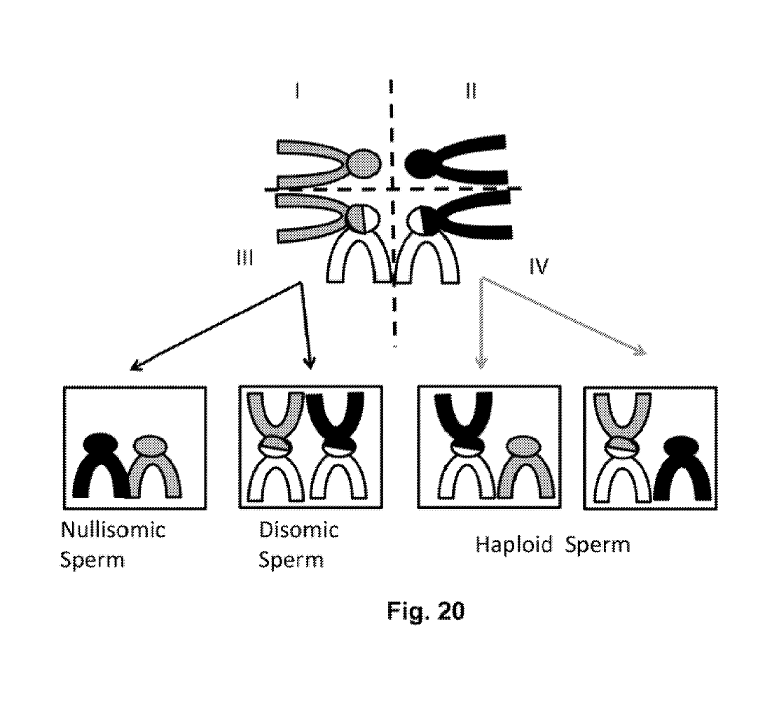

Generation of animals. Large numbers of mouse embryos with whole chromosomal aneuploidies were produced by using a sire that carries two Robertsonian (Rb) chromosomes, chromosomes formed by centromeric fusion of 2 chromosomes, with a common chromosomal arm, known as monobrachial homology. During meiosis, segregation between these two Rb chromosomes is impaired, leading to the production of gametes and embryos that are aneuploid (monosomic or trisomic) for the common arm chromosome as shown in FIG. 20. For this study, male mice doubly heterozygous for 3 pairs of Rb chromosomes with monobrachial homology for chromosomes 10, 11 and 15 were used to generate embryos. Fluorescent in situ hybridization of sperm from these males showed aneuploidy rates for the common arm chromosome of 35-44% with roughly half being nullisomic and half being disomic.

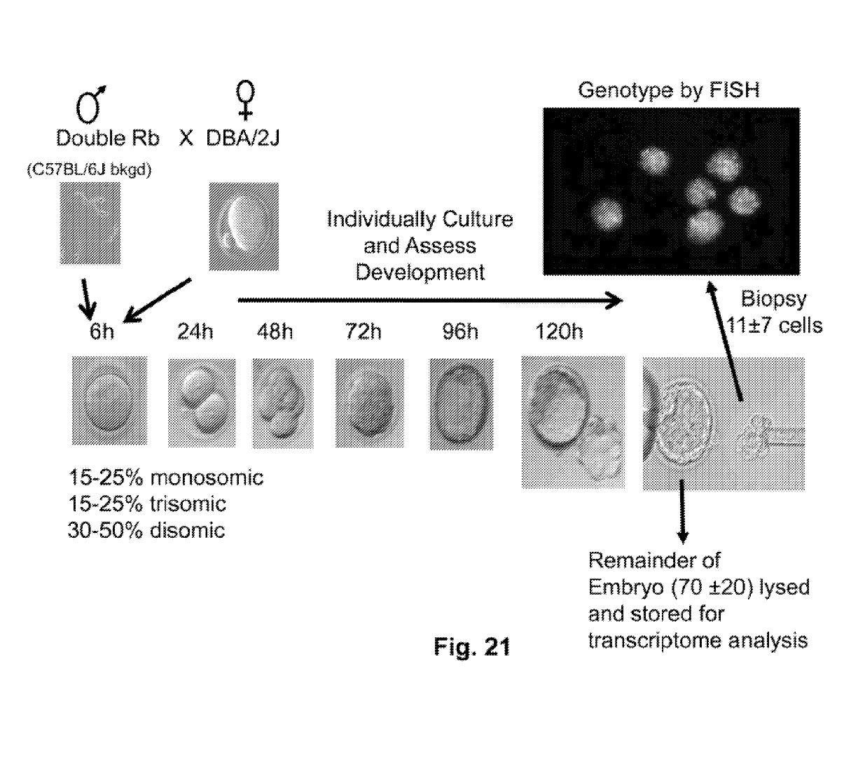

Embryo production, culture and biopsy. Embryos were generated by in vitro fertilization using cryopreserved sperm from males that carried the double Rb chromosomes in a C57B1/6J inbred background and oocytes from the DBA/2J inbred background (FIG. 21). Embryos were cultured individually in microdrops of a modified G series version 2 medium with daily morphologic assessment and culture medium changes. At 120 hours post-fertilization, 11+/−7 cells were removed from the mural trophectoderm of blastocysts using micromanipulator-controlled pipets and a Zylos-tk laser attached to an inverted microscope. The biopsy sample was processed for fluorescent in situ hybridization (FISH) using the protocol of Dozortsev and McGinnis ((2001) Fertil Steril 76: 186-8 incorporated herein by reference). The remainder of the blastocyst was placed into Arcturus Picopure Extraction buffer, flash frozen in liquid nitrogen and then stored at -80C until further processing.

Embryo genotyping. Biopsy samples fixed to slides were evaluated by FISH using BAC probes that anneal to the monobrachial chromosome as well as one other chromosome involved in the translocation using methods described by Scriven and Ogilvie (2010) Methods in Molecular Biology: Fluorescence in situ Hybridization (FISH) 659: 269-282. These probes were labeled with different fluorophores, and the biopsy samples were scored for signals from the two probes (first—from the Rb common arm chromosome and second from a chromosome on another Rb arm): 2/2-euploid, 3/2-trisomic, 1/2-monosomic, 3/3-triploid and mosaic when cells were present with different numbers of signals.

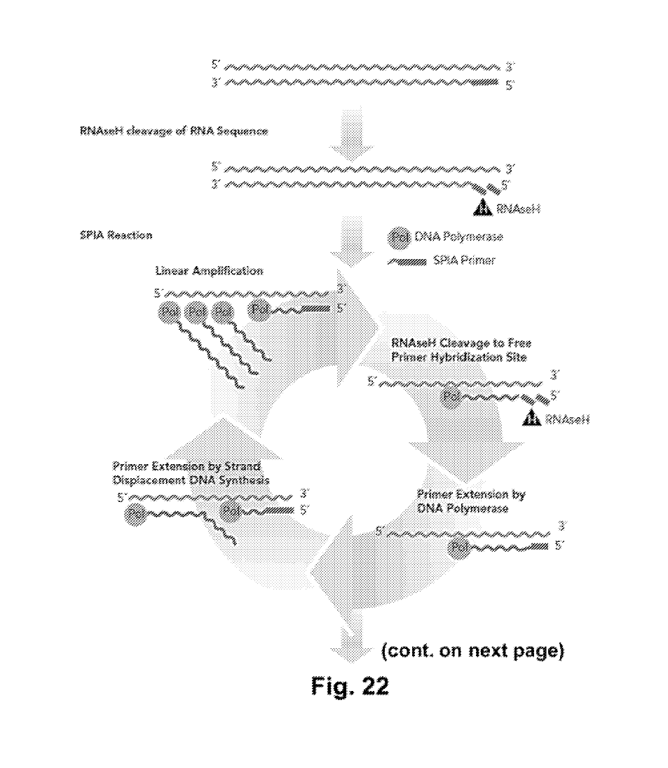

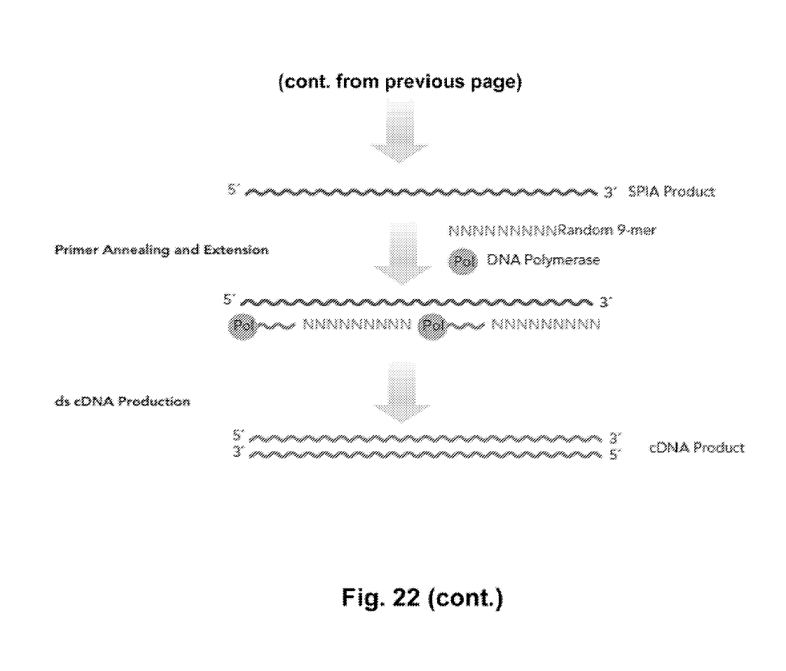

RNA-Seq sample preparation and sequencing. To evaluate the effects of the 3 trisomies on the transcriptome, 4-6 embryos of the same genotypes (disomic and trisomic) were pooled to serve as sources of RNA for this study (monosomic embryos were not evaluated because of insufficient numbers of embryos). Triplicate pools of disomic and trisomic embryos that were matched in terms of having the same number of embryos from the same IVF/culture run, the same parents, and similar developmental staging were generated for each of the 3 different trisomies. RNA was isolated using the Arcturus picopure kit per manufacturer's protocol, yielding 1-2 nanograms of high quality total RNA (RNA integrity number >8). Half of the RNA was amplified using the single primer isothermal amplification method (Nugen Ovation RNA-Seq kit) to generate amplified cDNAs (FIG. 22). This system produced over 4 micrograms of double-stranded cDNA from each sample. The cDNAs were fragmented with the Covaris adaptive focused acoustics system and libraries were prepared using the Nugen encore NGS library multiplex system 1. Libraries were generated with 4 different indexing tags to allow 4 libraries to be run per flow cell. Libraries were single-end sequenced on an Illumina HiSeq 2000 machine.

Sequence analysis. Sequence quality was assessed with FastQC version 0.10.0 (http://www.bioinformatics.babraham.ac.uk/projects/fastqc/). Reads were aligned to the mouse genome (mm9) with TopHat version 1.3.1 (Trapnell, et al. (2009) Bioinformatics 25: 1105-1111, incorporated herein by reference) using the default parameter settings. Differential expression was assessed using the Cuffdiff utility in Cufflinks (Trapnell, et al. (2012) Nat Protoc 7: 562-578; Trapnell, et al. (2010) Nat Biotechnol 28: 511-5, incorporated herein by reference) in conjunction with a locally developed perl script. Density, box, and scatter plots to confirm comparability of datasets were generated using the Cummerbund program in the Cufflinks package.

### Results

Impact of aneuploidies on embryonic development. Genotyping of blastocysts revealed that 15-22% were trisomic (comparable to sperm disomy rates of 22-25%). For the monosomies, there were significantly reduced number of monosomic embryos for chromosomes 10 and 11 as compared to the frequencies of trisomies, whereas there was no difference for chromosome 15 (12 vs 15%). A small fraction, 4-7%, of embryos were noted to be mosaic, with most being a mix of the aneuploid and euploid cells. In reviewing the developmental progression and morphology of embryos, it was also found that there was no appreciable difference in development or morphology between embryos with any of the 3 trisomies or monosomy 15 and wild type (euploid) embryos.

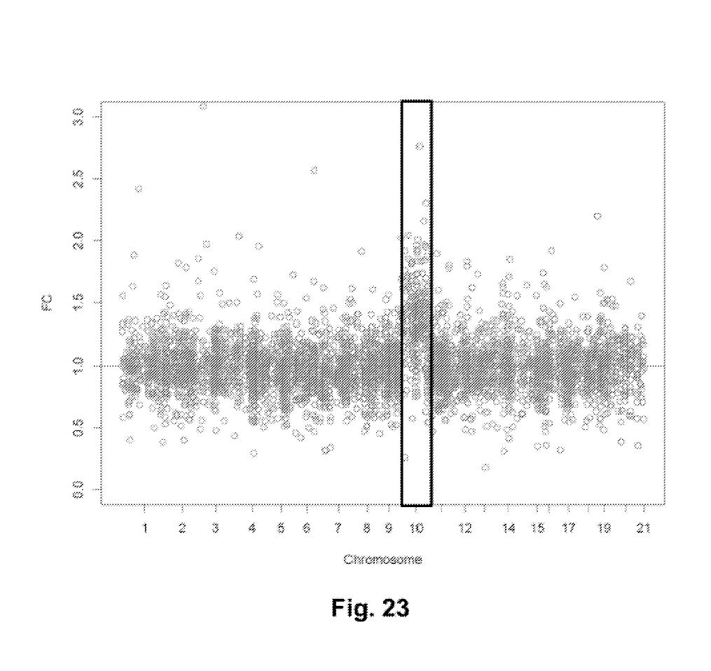

RNA-Seq Analysis. High throughput sequencing yielded on average 29.7 million 55-nucleotide reads per sample (min: 21.6 m, max: 38.6 m). QC analysis found all parameters assessed were good, with the exception of aberrant GC content and excess kmer content over approximately 10 bases at the 5′ ends of the reads. Based on this result, the first 10 bases from each read was trimmed using a locally developed perl script, yielding very high quality, 45-nucleotide reads for input to the aligner. Differential expression analysis using criteria of a fold change of greater than 1.5 and an FDR<0.05 found no differentially expressed transcripts for all 3 of the trisomies relative to the counterpart euploid samples. When the levels of expression of the transcripts on the trisomic chromosomes were compared to expression levels of the same loci in disomic samples, it was found that a significantly high fraction, exceeding 90% of transcripts, were overexpressed relative to disomic samples (χ-square<0.001). In contrast, there was no difference in levels of expression for nontriplicated loci between trisomic and disomic samples. The median/mean fold-change in expression for loci on the trisomic chromosome relative to expression levels of these loci in disomic samples was around 1.4 for all 3 trisomies. A graphical presentation of these fold changes for trisomy 10 is shown in FIG. 23.

### Discussion:

Genotypic analyses of embryos reveal that there was no selection against sperm or embryos with the 3 trisomies and monosomy 15 throughout the preimplantation period whereas the other 2 monosomies were compromised in their ability to develop throughout the preimplantation period. These findings support the clinical observation that trisomies often do not compromise preimplantation development whereas monosomies can. These findings also highlight the fact that, like with human embryos, mouse embryos with substantial genomic abnormalities that are not compatible with prenatal development can develop essentially normally throughout the preimplantation period. These finding suggest that morphologic and developmental assessments have poor predictive value in identifying embryos with at least some genomic imbalances, including select trisomies.

The findings of no differentially expressed loci between trisomic and disomic RNA-Seq samples reveals that the standard means of assessing differential expression are too stringent for identifying primary or secondary perturbations in the transcriptome caused by aneuploidies. In some cases, aneuploidies can cause relatively small magnitude changes that cannot be detected in small datasets.

The high proportion of transcripts from the trisomic chromosome that are upregulated by approximately 1.5-fold indicates that there is a very strong correlation between copy number and transcript expression level in the preimplantation period, perhaps even higher than in most other cell types. In contrast, studies of CNVs in postnatal tissues from mice found only 5-18% of loci to show a strong positive correlation with copy number (Henrichsen (2009) Hum Molec Genet 18: R1-8).

### XI.B. Example 2

### Transcriptome Analysis of Human Lymphoblast Cells that Carry a Deletion

Results of analyses of RNA-Seq data from a human lymphoblast line carrying a 34 Mb deletion of chromosome 21 are presented. The interstitial deletion removes about 70% of the chromosome. This study includes analysis of data from samples generated from both a large amount of input material as well as an amount of input material comparable to the amount that would be present in a typical blastocyst biopsy. The goals of this study are two-fold: (1) assess the impact of this deletion on the transcriptome using the large input sample and (2) determine if any observed expression alterations can be detected in a low input sample.

### Methods

Cell culture. Three lymphoblast cell lines derived by EBV transforming peripheral lymphocytes from different individuals were obtained from Coriell: (1) GM10857, a female line with no detectable large copy number alterations, (2) GM10851, a male line with no detectable large copy number alterations and (3) GM01201, a female line that carries a 33.6 Mb deletion extending from 13322592-46921373. Cell lines were cultured as recommended from Coriell. Briefly, cells were cultured in suspension in RPMI 1640 culture media containing 2 mM L-glutamine and supplemented with 15% fetal bovine serum at 37 C with 5% CO₂. Cells were seeded at a density of 200,000 viable cells/ml and cultured for 3-4 before being split 1:3 or 1:4.

Sample preparation. For large input samples, four replicates of 20,000 cells were collected from the suspension culture from each cell line. Samples were washed three times in PBS without magnesium and calcium and containing 5% molecular biology grade bovine serum albumin and then resuspended in Prelude™ Direct Lysis Module (NuGEN Technologies, Inc.; San Carlos, Calif.). Lysates were snap frozen in liquid nitrogen immediately after resuspension and then stored at −80 C for further processing. To prepare samples containing a smaller number of cells for line GM01201, flow sorting was used. Briefly, cells from each of the 5 lines were washed 3 times and then resuspended in the previously described PBS-BSA solution. Immediately before sorting, propidium iodide was added to the sample for a final concentration of 1 μg/ml. Cells were then sorted using a 4 laser BD FACS Aria flow sorter. Cells were first analyzed based on forward scatter versus side scatter. A gate for live cells was made based on forward scatter, which measures a cell's size, and side scatter, which measures a cell's complexity or granularity. A further exclusion for dead cells was done using PI positive cells, and a gate was placed around the PI negative cell, for the sort collection. PCR tubes containing 2 μl of Prelude lysis buffer were placed in a modified Terasaki plate on the ACDU plate collection unit. Counts of cells aliquotted using these conditions into optical plates revealed that wells had 4-10 cells/well. This cell number is comparable to the number obtained from an embryo biopsy.

cDNA synthesis and amplification. Lysates from large and small input lysates from each line were used for cDNA and amplification. The Ovation® RNA-Seq system (NuGEN Technologies, Inc.; San Carlos, Calif.) was used for cDNA generation and amplification per manufacturer's recommended protocols and as described previously in Tariq, et al. ((2011) Nucleic Acids Res 39: e120, incorporated herein by reference). Briefly, total RNA in the lysate was reverse-transcribed to first-strand cDNA using a combination of random hexamers and poly-T chimeric primers and then converted to double-stranded (ds) DNA using fragmentation and RNA-dependent DNA polymerase. Finally, the ds cDNA was amplified linearly using a single primer isothermal amplification process (FIG. 22) and purified by using MyOne™ carboxilic acid-coated superparamagnetic beads (Invitrogen, Carlsbad, Calif.). The quality and quantity of cDNA were evaluated using the Agilent Bioanalyzer 2100 DNA High Sensitivity chip (Agilent; Palo Alto). All samples generated sufficient cDNA.

Library preparation and sequencing. Approximately 0.5-1.0 μg of amplified cDNA from each sample was sheared to a size ranging between 300-500 bp using the Covaris-S2 sonicator (Covaris, Woburn, Mass.) according to the manufacturer's recommended protocols. Fragmented cDNA samples were used for the preparation of RNA-Seq libraries using TruSeq v1 Multiplex Sample Preparation kit (Illumina, San Diego, Calif.). Briefly, cDNA fragments were end-repaired, dA-tailed and ligated to multiplex adapters according to manufacturer's instructions. After ligation, DNA fragments smaller than 200 bp were removed with AmPure XP beads (Beckman Coulter Genomics, Danvers, MA). The purified adapter ligated products were enriched using polymerase chain reaction (14 cycles). The final RNA-Seq libraries were quantitated using the Agilent bioanalyzer 2100 and pooled together in equal concentration for sequencing. The pooled multiplexed libraries were sequenced with 2 sample being run per lane, generating 50 by paired-end reads on HiSeq 2000 (Illumina, Inc; San Diego, Calif.). Data analysis. Reads from all samples were checked for quality and preprocessed prior to alignment. Fastqc is used to determine overall quality of the sequencing run and checks for drops in 5′ or 3′ ends of reads, overrepresentation of k-mers such as homopolymers or sequencing adapters, shifts in expected GC content and excessive duplication rate. Datasets with low quality scores in the 5′ or 3′ ends of reads were corrected by trimming reads using the fastx toolkit. Datasets with an overrepresentation of sequencing adapters were also corrected by trimming sequencing adapter sequence from 3′ ends of reads or removing reads containing sequencing adapters.

Data analysis. Preprocessed reads were aligned to a transcriptome generated from the UCSC hg19 human reference sequence and the UCSC knownGene annotation. STAR was used to generate spliced alignments in BAM format. Alignments were then sorted and indexed using samtools. Alignments were further postprocessed to remove PCR duplicates (reads determined to have the same starting and ending location for forward and reverse reads) and to report only uniquely mappable reads using samtools. Datasets are further QC'd using RSEQC to check for biases in coverage, exonic enrichment, and to generate RPKM estimates for all genes. Expression estimates were further checked for quality by generating pairwise Spearman's correlations between samples. Samples with Spearman correlations of less than 0.7 were not used for further analyses.

To assess the impact of a copy number alteration on regional expression of the genome, the general approach previously outlined for locus expression based CNA was used. First, the expression data for a single sample was compared to a reference. The reference used was the median expression values generated from expression data from large input samples excluding the sample that was being analyzed. The expression value for each locus in the sample was divided by the respective reference expression level to generate a fold change. Using predetermined regions of whole chromosomes, the relative expression of each autosome relative to other autosomes was evaluated using a two-sided Wilcoxon rank sum test. In this test, the distribution of fold change values for each autosome was compared to the fold change values in all other autosomes (i.e., chromosome 1 distribution was compared to all other autosomes, then chromosome 2 distribution compared to all other autosomes, etc). P-values generated for each chromosome were then adjusted for multiple testing using the Bonferroni correction.

### Results

Data generation. Of the samples that met the QC criterion, 5 large input (two from each euploid line and one from GM02101) and one small input from GM01201 were analyzed, allowing the effect of the deletion in GM01201 to be assessed in both large and small input samples.

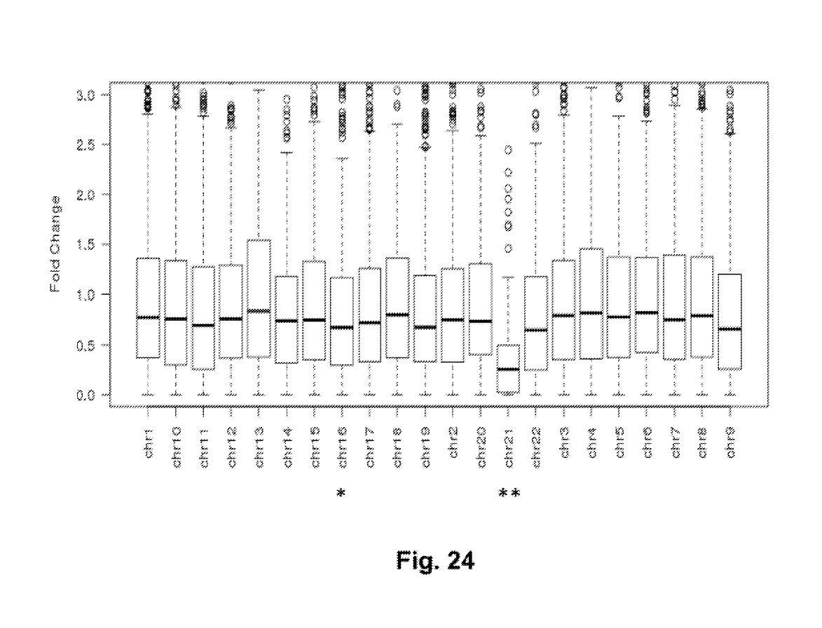

Analysis of large population data. All 5 high input samples had high correlation to median expression values with Spearman's correlation of R>0.94, indicating that, as expected, these expression profiles from these cell lines are highly similar despite originating from different individuals and containing different CNAs. In looking at the relative expression for the GM01201 sample, it was found that most chromosomes had similar patterns of expression with the exception of chromosome 21, which had markedly reduced expression (FIG. 24). When evaluated with the Wilcoxon rank sum test, it was found that most autosomes had a p-value of around 1 with the exception of autosomes 6 (p=0.008), 9 (p=0.10),16 (p=0.18), 22 (p=0.032) and 21 (p=6.9×10⁻²⁷). The mean coefficient of variation for the fold changes of chromosomes is 3.1±1.1.

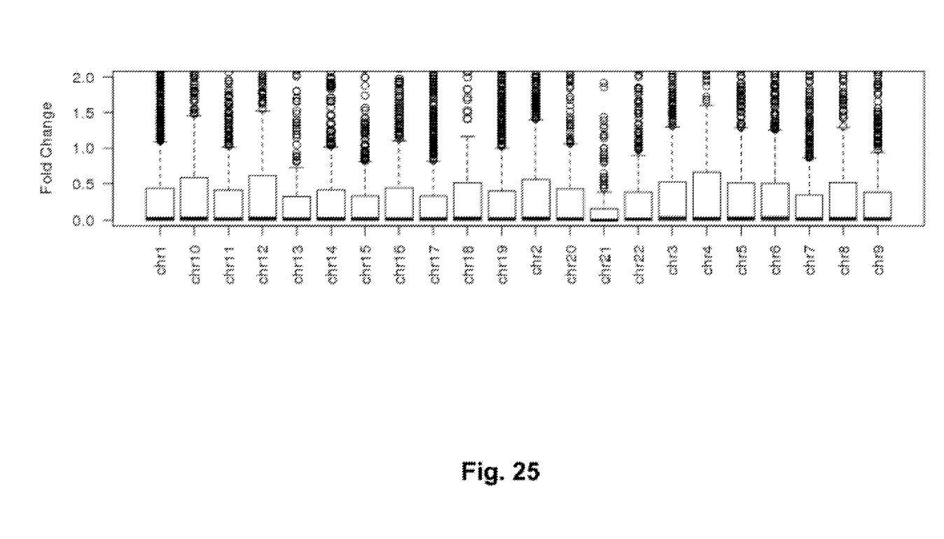

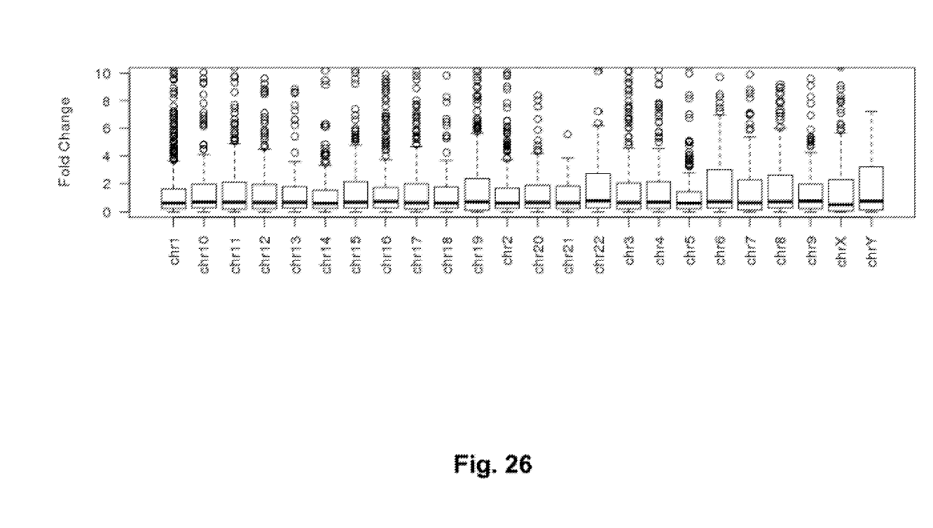

Analysis of the small population data. The Spearman correlation between GM01201 and median RPKM values of the reference showed a correlation of 0.71. When the relative expression of the autosomes were examined, chromosome 21 was found to have a reduced upper quartile relative to other chromosomes (FIG. 25). The Wilcoxon rank sum test showed most chromosomes to have p values around 1 with the exception of autosomes 2 (p=0.95), 12 (p=0.97) and 21 (p=0.0018). The average coefficient of variation for fold changes of the chromosomes was 11.9±3.6.

### Discussion

The expression data from the large input sample for line GM01201 shows that the deletion, which removes more than 70% of chromosome 21 leads to a generalized reduced expression of this chromosome, as supported by the very low p value from the rank sum test analysis. This finding indicates that a substantial proportion of loci on chromosome 21 are dosage sensitive and have positive correlations with copy number. When the small input sample from this line was evaluated, a similar reduction in expression of chromosome 21 was noted. Once again, the relative expression of this chromosome was significantly reduced as compared to other chromosomes as attested to by the low rank sum test p value. By using a threshold based on p value, this segmental aneusomy can be identified in a few cells using this analytic approach.

### XI.C. Example 3

### Evaluation of RNA-Seq Data from Human Embryos

In this example, publically available RNA-Seq data generated from mural trophectodermal cells from 2 human blastocysts are analyzed. The goals of this study are to compare the data to the lymphoblast data from a small number of cells and compare the two samples to see if there is any evidence of a copy number alteration.

### Methods.

Sample collection and data generation. The methods used to generate the data are described in detail in the report by Yan et al ((2013) Nat Struct Mol Biol 20: 1131). Briefly, single cell samples were collected from dissociated blastocysts and transferred into lysis buffer. The protocol for generation of RNA-Seq data from these lysates is described in Tang et al (2010) Nature Protocols 5: 515, incorporated herein by reference). Briefly, this approach involves the generation of cDNA using an oligo(dT) primer, polyadenylating the first strand with terminal transferase, priming the second strand with an olig(dT) primer and then PCR amplification of the cDNAs using a universal primer. Data were generated from five cell lysates (cells 4, 6, 7, 9, and 12) collected from blastocyst #1 and four cell lysates (cells 4, 5, 6 and 7) collected from blastocyst #2. Raw data from this experiment were downloaded from SRA Submission SRA050912.

Data analysis. Reads from all samples were aligned to a transcriptome generated from the UCSC hg19 human reference sequence and the UCSC knownGene annotation. STAR is used to generate spliced alignments in BAM format. Alignments are then sorted and indexed using samtools. Alignments are further post-processed to remove PCR duplicates (reads determined to have the same starting and ending location for forward and reverse reads) and to report only uniquely mappable reads using samtools. 15 million mapped reads were randomly sampled from each sample and combined to simulate a run in which a single library was prepared for 4-5 cells. RPKM estimates for UCSC knownGenes were generated for each simulated 4-5 cell trophectoderm biopsy. Fold change values were calculated for each locus by dividing simulated embryo 1 by simulated embryo 2. Evaluation of alterations in relative expression for the autosome and X chromosome were performed as described previously in Example 2.

### Results

Spearman correlation between the two simulated embryo biopsies was 0.87. Boxplots of fold change show similar distributions for all chromosomes with the exception of the X chromosome, which has a lower median. Wilcoxon rank sum analysis revealed that all autosomes and the Y chromosome had p values of around 1, with the exception of chromosome 16 (p=0.45). In contrast, the X had a markedly lower p value (0.00019) due to its lower median. The coefficients of variation for the chromosomal relative expression data averaged 6.7±1.8.

### Discussion

In assessing the quality metrics of these data as compared to those of the low input sample in Example 2, the Spearman correlation (0.87 vs 0.71) and coefficients of variation for the fold changes (6.7±1.8 vs 11.9±3.6) indicate that that the quality of sequence data that can be generated from embryo samples is as good, if not better, than the low input sample that was used to detect a segmental aneusomy in Example 2. The finding of relatively comparable expression profiles for all of the autosomes is consistent with there being no aneuploidy in either embryo. Given that the 2 embryos for this study were generated from women age 30-35 years, it would expected that only around 30% of embryos would be aneuploid (Harton et al (2013) Fert Steril 100: 1695-1703, incorporated herein by reference). The finding of a significantly lower distribution for the X chromosome in embryo 1 indicates that embryo 1 is likely to have one X chromosome or have 2 X chromosomes with one harboring a large interstitial deletion and embryo 2 is likely to have 2 X chromosomes. The most likely explanation is that embryo 1 is male, and embryo 2 is female. It was not confirmed that embryo 1 is male based on Y chromosome expression due to the very low expression of the Y and the possibility of reads being erroneously mapped to the Y chromosome. In analysis of expression data of female lymphoblast lines in Example 2, it was found that the Y chromosome had a substantial number of aligned reads. Expression data from confirmed male and female blastocysts can be used to develop appropriate filters to enable evaluation of Y chromosomal expression.

### XI.D. Example 4

### Clinical Detection of Aneuploidy with RCNAD

In this prophetic example, established approaches for generating RNA-Seq data from single cells and algorithms for identifying CNAs are applied in a clinical scenario. In this example, a father age 47 and a mother age 42 who have a 2-year history of 4 miscarriages are undergoing IVF and transcriptome-based CNA screening to reduce the chances of having an aneuploid pregnancy. Prior workup for recurrent miscarriages, including karyotypic analysis of both parents, is normal.

### Methods

Embryo generation and sample acquisition. Embryos are generated by standard ART procedures performed in a CLIA-certified ART laboratory, including controlled ovarian hyperstimulation, oocyte retrieval by follicular aspiration, fertilization by ICSI and culture of embryos to the blastocyst stage. A total of 14 oocytes are collected and 11 proceed to develop. On the 3^{rd} day of culture, the zona pellucida is breached in each developing embryo. On the 5^{th} day of culture, 9 hatching or fully expanded blastocysts are transferred to individual, labeled microdrops on low profile biopsy dishes containing microdrops of G-MOPs overlaid with Ovoil. A herniated piece of trophectoderm from a hatching blastocyst or a piece of mural trophectoderm from an expanded blastocyst containing 5-10 cells is obtained using a Xylos tk laser and polar body biopsy pipets (Humagen). Immediately following biopsy, the blastocyst is transferred back to culture medium and returned to an incubator to continue the culture. Following completion of biopsies and processing of all biopsy specimens, embryos are cryopreserved using a standard vitrification technique.

RNA isolation and spike in control addition. Immediately after biopsy, each biopsy specimen is washed three times through phosphate-buffered saline containing 5 mg/ml molecular biology grade bovine serum albumin using a 50 micron inner diameter stripper pipet tips and a Human PGD stripper micropipetter. Each washed biopsy sample is then placed in 3 microliters of hypotonic lysis buffer comprising of 0.2% Triton X-100 and 2 U/microliter of ribonuclease (RNase) inhibitors (Clontech, 2313B) in RNase free water in 0.2 microliter non-stick, RNAse-free, tubes (Ambion). This reaction buffer is included in the Clontech SMARTer™ Ultra Low RNA Kit. To each sample, 1 microliter of lysis buffer containing 10,000 copies of ERCC spike in synthetic RNA (Life Technologies) is added. Samples are then either snap frozen in liquid nitrogen or immediately processed for transcriptome analysis. Snap frozen samples are stored at −80 C or colder temperatures until subsequent processing.

Production of double-stranded cDNA. This protocol uses the SMART-Seq protocol developed by Ramskold et al ((2012) Nature Biotech 30: 777-82, incorporated herein by reference) and available as a commercial kit, the SMART-Seq Ultralow RNA Kit for Illumina Sequencing (Clontech). Samples are prepared and analyzed in a CLIA certified, CAP accredited laboratory. Both the first and second strands of cDNA are synthesized simultaneously using the template strand switching approach (Zhu, et al. (2001) Biotechniques 30: 892-897, incorporated herein by reference). For this process, an oligodT tailed cDNA synthesis primer (5′-AAGCAGTGGTATCAACGCAGAGTACT(30)VN-3′ (SEQ ID NO: 1), where V represents A, C or G), a SMARTer II A oligo (5′-AAGCAGTGGTATCAACGCAGAGTACATrGrGrG-3′₋(SEQ ID NO: 2), where r indicates ribonucleotide bases), 5x First Strand Buffer (250 mM Tris-HCl pH 8.3, 375 mM KCl and 30 mM MgCl₂), dithiothreitol (100 mM), dNTP mix (10 mM), RNAse inhibitor, oligos (CDS primer and SMARTer II A oligo) and 100U SmartScribe Reverse Transcriptase are combined in a total volume of 10 microliters. In this reaction, after completing the oligo(dT) primed first strand, MMLV, through its terminal transferase activity, adds a polycytosine tract to the strand. The SMARTer II Oligo anneals to this polycytosine tract and primes extension of the second strand (see e.g., FIG. 11). The resulting full-length cDNA contains the complete 5′ end of the mRNA as well as an anchor sequence that serves as a universal priming site for second-strand synthesis. Following cDNA synthesis, the products are purified using SPRI Ampure Beads. The reagents for this method are available in the Clontech SMARTer™ Ultra Low RNA Kit.

cDNA Amplification. Double stranded cDNA produced by the SMARTer technology contains sequences at each end of the cDNA that serve as universal priming sites for amplification by PCR. PCR-based amplification is performed using the long-distance PCR kit, Advantage 2 (Clontech) with PCR primer (5′-AAGCAGTGGTATCAACGCAGAGT-3′ (SEQ ID NO: 3)) and thermocycling conditions: 15 cycles of 95° C. for 15 seconds, 65° C. for 30 seconds and 68° C. for 6 minutes. The amplification products are evaluated using a nanodrop spectrophotometer and the Agilent 2100 BioAnalyzer using the nanochip. All samples have 2-7 nanograms of DNA with the predominant species ranging in size from 400-9000 bp with a peak at approximately 2000 bp as expected.

DNA Fragmentation. DNA is fragmented using the Nextera technology, which utilizes a tn5 transposase to simultaneously fragment the double-stranded DNA and ligate adapters to the ends of the fragments (see e.g., FIG. 12). With the Tn5 protocol, the amplified cDNA is ‘tagmentated’ at 55° C. for 5 min in a 20-μl reaction with 0.25 μl of transposase and 4 μl of 5× HMW Nextera reaction buffer (containing Illumina-compatible adapters). To strip the transposase off the DNA, 35 μl of PB buffer is then added the tagmentation reaction mix, and the tagmentated DNA is purified with 88 μl of SPRI XP beads (sample to beads ratio of 1:1.6). The reagents for this method are available in Nextera DNA sample kits (Epicentre/Illumina).

Library production. Libraries are prepared for sequencing using the Illumina platform. Limited-cycle PCR with a four-primer reaction adds bridge PCR (bPCR)-compatible adaptors to the core library (used for binding fragments to the flow cell). By including different Illumina compatible bar codes between the downstream bPCR adaptor and the core sequencing library adaptor in sets of 4 samples, 12 samples on the same flow cell can be run. The bPCR/barcode/sequencing adapters are added to the library by incubating the reactions at 72° C. for 3 minutes followed by 9 cycles of: 95° C. for 10 seconds; 62° C. for 30 seconds and 72° C. for 3 minutes. The reagents for this step are included in the Nextera DNA Sample Prep Kit (Illumina-compatible). Following amplification, library quality is confirmed using DNA 1000 kits on an Agilent Bioanalyzer. All 9 samples pass the QC analysis.

Sequencing. Twelve samples are run per flow cell on the Illumina HiSeq 2000 system, generating about 10 million paired reads/sample. In a report using this method for single cell RNA-Seq, it is found that at above 3 million uniquely mapping reads, there is little impact on transcript detection (Ramskold, et al. (2012) Nat Biotechnol 30: 777-82, incorporated herein by reference).

Quality assessment and data filtering. FastQC version 0.10.0 is used to assess quality per sequence and per base (phred scores); GC and N content; sequence length distribution, overrepresented sequences, sequence duplication levels and kmer content. Based on these quality scores, poor sequences and/or segments of sequence are culled. A comparison of expected to observed concentrations for ERCC spike in reveals that all 9 samples have Spearman correlations of >0.9. All 9 samples are deemed to be of sufficient quality for further analysis.

Sequence alignment and depth of coverage assessment. Novoalign from Novocraft Short Read Alignment Package (http://www.novocraft.com/index.html) is used to align each lane's SEQ file to the reference genome. Human Genome reference sequence (GRCh38, Release date: Dec. 24, 2013), is indexed using novoindex program (-k 14 -s 3). The output format is set to SAM and default settings are used for all options. Using SAMtools (http://samtools.sourceforge.net/), the SAMfiles of each lane are converted to BAM files, sorted and merged for each sample and potential PCR duplicates are removed using Picard (http://picard.sourceforge.net/). To retrieve the depth of coverage information of each base, a PILEUP file for each sample is generated using SAMtools and the average coverage per capture interval is calculated using a custom script.

SNP genotyping and haplotype analysis Before identifying heterozygous SNPs in the genome, the depth of coverage for each base, a parameter in determining the confidence for calls is calculated from a PILEUP file generated by SAMTools software. Variant sites are then called by the Genome Analysis Toolkit software (McKenna, et al. (2010) Genome Res 20: 1297-1303, incorporated herein by reference). To determine haplotypes in the embryo, parental genomic DNA is isolated from peripheral blood samples using the QIAmp DNA mini blood kit (Qiagen) and genotyped using an Illumina custom SNP microarray that is developed to genotype all SNPs in coding regions of all transcripts expressed in human embryos. The parental and embryo SNP data are used to generate parental linkage haplotype data for each embryo using Triocaller software (Chen, et al. (2013) Genome Research 23: 142-151, incorporated herein by reference).

CNA Identification using locus expression data. CNAs are identified using ExomeCNV (Sathirapongsasuti, et al. (2011) Bioinformatics 27: 2648-2654, incorporated herein by reference). This program uses a normalized depth of coverage ratio to evaluate the relative expression at the exon level of the sample as compared to a reference. The reference for this analysis is composed of median read counts for each exon obtained from a large dataset of embryonic samples generated in the same manner as the test sample. Using ExomeCNV, a CNA in an exon is identified by a deviation of a transformed ratio from the null, standard normal distribution that is beyond empirically defined thresholds defined using aneuploid and embryos. Once exons are evaluated, the exonic data are combined into segments using circular binary segmentation (CBS). Copy number status is assigned using empirically derived thresholds.

Evaluation of allelic expression data. A slightly modified version of ExomeCNV is also used to evaluate SNP data from the embryo's transcriptome to look for evidence of CNAs and loss of heterozygosity. In this example, SNP data in the transcriptome are predominantly parental linkage phased, meaning that for most SNPs, it is known which SNP alleles are associated with which parental chromosome and also which SNPs are expected to be heterozygous. In this analysis, the relative expression of the parental alleles for all expected and experimentally detected heterozygous SNPs (i.e., SNPs that are predicted to be heterozygous based on the 2 haplotypes present and any SNPs that experimentally have at least 5 reads for each allele) are compared to similar ratios from parental linkage haplotyped reference data. The reference ratios represent the median ratios from a large dataset of embryo samples generated in a similar fashion. By comparing the sample ratios to the reference, it will be possible to assess the relative expression of the parental alleles of loci. Analysis will be performed by comparing the read count for the paternal and maternal alleles of the sample to the expected counts derived from the paternal: maternal expression ratio of the reference using a binomial test. Once SNPs are evaluated, segments can be combined using the deviation of the ratio (ratio of the sample-ratio of the reference) using circular binary segmentation (CBS). By looking at the magnitude of the alteration in ratio and whether polymorphisms in the affected region are mono- or bi-allelic will help to indicate the type of CNA is most likely present on which parental chromosome. To distinguish LOH arising from a deletion from that which arises from uniparental disomy, locus or allelic expression data can be evaluated.

Evaluation of breakpoints. To search for breakpoints, the FusionQ analytic package is used, which has been developed for RNA-seq data (Liu et al (2013) BMC Bioinformatics 14: 193, incorporated herein by reference). This tool can detect gene fusions, construct the structures of chimeric transcripts, and estimate their abundances. To confirm the read alignment on both sides of a fusion point, a residual sequence extension approach is used, which extends the short segments of the reads by aggregating their overlapping reads. A list of filters is also included to control the false-positive rate. Fusion transcript abundance is estimated using the expectation-maximization algorithm with sparse optimization.

Evaluation of expression signatures. In this prophetic example, an expression signature for trisomies is available based on analysis of a large dataset of samples from embryos with trisomies using previously described methods for expression signature identification. This signature includes 64 loci, with 47 being upregulated and 17 being down regulated. A scoring method is developed based on the relative expression of these loci in which the relative expression of each locus is weighted by a factor reflecting the frequency of the alteration in expression of this locus across the trisomies and then all values are summed. The total is then assigned a risk of low, medium or high risk based on empirically derived cutoffs. Expected results

The results for RCNAD analyses of the 9 embryos are shown in Table I. For locus expression based CNA detection (LECNAD), screening reveals evidence for 3 embryos with trisomies and 2 embryos with monosomies. Allele expression based analysis (AECNAD) finds imbalances in the paternal: maternal allele expression for all aneuploidies. Of note, 5 of the 6 aneuploidies are of maternal origin (i.e., trisomy decreases P:M ratio and monosomy increases P:M ratio). Trisomy 6 in embryo 5 appears to be of paternal origin due to the direction correlation with P:M ratio. Breakpoint identification CNA detection finds no evidence of gene fusions. Signature expression-based CNA detection finds that all trisomies have a high risk profile for trisomy, whereas those embryos with monosomies or without evidence of CNAs have low risk with the exception of embryo 7.

TABLE I

| Test | Emb 1 | Emb 2 | Emb 3 | Emb 4 | Emb 5 | Emb 6 | Emb 7 | Emb 8 | Emb 9 |
|---|---|---|---|---|---|---|---|---|---|
| LECNAD | No | +16 | +22 | No | +6 | No | No | −4 | −18 |
| | CNA | | | CNA | +21 | CNA | CNA | | |
| AECNAD | No | ↓ 16 | ↓22 | No | ↑ 6 | No | No | ↑4 | ↑18 |
| (P:M) | imb | | | imb | ↓21 | imb | imb | | |
| BICNAD | None | None | None | None | None | None | None | None | None |
| SECNAD | Low | High | High | Low | High | Low | Mod | Low | Low |
| Plan | Tfer | Res | Res | Cryo | Res | Cryo | Cryo | Res | Res |

The results from the RCNAD analyses are conveyed to the ordering physician and after consultation with the family, it is decided that only one of the embryos without evidence of CNAs and a low trisomy risk estimate from the trisomy signature panel (i.e., embryo 1) will be warmed and transferred during a natural cycle. The remaining 3 embryos without expression evidence for CNAs are maintained in cryopreservation for potential future transfers. The decision to keep embryo 7 with the moderate trisomy risk from SECNAD screening is made with the understanding that this score increases the risk of a pregnancy loss or trisomic fetus by several fold based on data from the clinic. The five cryopreserved embryos with evidence of CNAs are donated to research.

### XI.E. Example 5

### Detection of a Segmental Aneusomy with RCNAD

In this prophetic example, embryos are screened for genomic consequences of a parent who carries balanced translocations involving chromosomes 12 and 21 (t(12;21)(p13;q22) and t(21;12)(q22;p13)). The father who carries these translocations had acute lymphoblastic leukemia as a child, partially the result of the fusion locus resulting from the fusion of ETV6 exon 5 sequences joined to exon 2 of sequences of AMLJ. This translocation is the most commonly recognized structural chromosomal abnormality in pediatric cancer cases. Unbalanced products of this translocation can lead to gains or losses of approximately 12 Mb of the p arm of chromosome 12 and 12 Mb of the q arm of chromosome 21.

### Methods

The methods for embryo generation and sampling and RCNAD are performed as outlined in Example 4. A total of 16 oocytes are collected, and 7 embryos develop to the blastocyst stage and are biopsied.

### Expected Results

The results of RCNAD are shown in Table II. LECNAD shows 3 of the embryos to have segmental aneusomies as a result of inheritance of unbalanced translocations. Two embryos have aneuploidies. AECNAD confirms the imbalances and aneuploidies, demonstrating that the segmental imbalances are inherited from the father and the aneuploidies from the mother. BICNAD finds the expected ETV6-AML1 gene fusion in the two embryos that carry this chromosome. One of the embryos without evidence of a CNA is found to have this gene fusion, indicating that this embryo is a balanced carrier for the translocations. SECNAD finds only high risk of trisomy for the embryo with evidence for trisomy 14.

TABLE II

| Test | Emb 1 | Emb 2 | Emb 3 | Emb 4 | Emb 5 | Emb 6 | Emb 7 |
|---|---|---|---|---|---|---|---|
| LECNAD | −12p | +14 | −12p | No | No | +12p | No |
| | +21q | −5 | +21q | CNA | CNA | −21q | CNA |
| | | | −X | | | | |
| AECNAD | ↓12p | ↓14 | ↓12p | No | No | ↑12p | No |
| (P:M) | ↑21q | ↑5 | ↑21q | imb | imb | ↓21q | imb |
| | | | ↓X | | | | |
| BICNAD | +12p; | None | +21p; | None | +21p; | None | No |
| | 21q | | 21q | | 21q | | imb |
| SECNAD | Low | High | Low | Low | Low | Low | Low |
| Plan | Res | Res | Res | Tfer | Cryo | Res | Cryo |

The results of the above tests are transmitted to the medical staff and parents. The parents and staff decide to transfer one of the embryos that has no evidence for a CNA and does not carry the detectable translocation. The other embryos without CNAs are cryopreserved for consideration of future use. The embryo with the balanced translocation is considered to have the lowest indication for transfer as a result of the increased risk for cancer. The embryos with segmental aneusomies and/or aneuploidies are donated to research.

### XI.F. Example 6

### Detection of Uniparental Disomy with RCNAD

In this prophetic example, a female carrier of a 13;14 Robertsonian translocation and her husband are referred for preimplantation genetic diagnosis after over 4 years of trying to have a child. Carriers of this translocation are at high risk of having aneuploidies of chromosomes 13 and 14, many of which are not compatible with development through the full prenatal period. The couple chooses to undergo RCNAD to increase their chances of establishing a chromosomally normal pregnancy.

### Methods

The methods for embryo generation and sampling and RCNAD are performed as outlined in Example 4. In this example, 9 embryos are biopsied and cryopreserved.

### Expected Results

LECNAD finds 5 embryos to have aneuploidies associated with the translocation. Three embryos have aneuploidies involving other chromosomes and one has a segmental aneusomy involving chromosome 16. AECNAD confirms all aneuploidies and segmental aneusomies and shows that all are inherited from the mother. In embryo 5, there is no evidence of paternal alleles for chromosome 14, suggesting that this embryo has maternal uniparental disomy, most likely arising as a result of trisomy rescue. BICNAD finds no breakpoints, indicating that the breakpoint associated with the 16q deletion in embryo 6 is not located within an expressed locus. SECNAD results are consistent with LECNAD and AECNAD analyses.

TABLE III

| Test | Emb 1 | Emb 2 | Emb 3 | Emb 4 | Emb 5 | Emb 6 | Emb 7 | Emb 8 | Emb 9 |
|---|---|---|---|---|---|---|---|---|---|
| LECNAD | +13 | −14 | No | −13 | No | No | +13 | −4 | −14 |
| | +17 | +18 | CNA | +16q | CNA | CNA | | | |
| AECNAD | ↓13 | ↑14 | No | ↑13 | ↓↓14 | No | ↓13 | ↑4 | ↑14 |
| (P:M) | ↓17 | ↓18 | CNA | ↓16q | | CNA | | | |
| BICNAD | None | None | None | None | None | None | None | None | None |
| SECNAD | High | High | Low | Low | Low | Low | High | Low | Low |
| Plan | Res | Res | Cryo | Res | Res | Tfer | Res | Res | Res |

Based on these results, the parents and healthcare team decide to transfer one of the 2 embryos without CNA or UPD. The other embryo is maintained in cryopreservation. The other embryos are donated to research.

### XI.G. Example 7

### Screening for a Single Locus Disorder in Concert with RCNAD

In this prophetic example, a male with congenital bilateral absence of the vas deferens and his wife are planning to undergo preimplantation genetic screening for mutations in the cystic fibrosis gene (CFTR). Absence of the vas deferens causes male infertility and can be caused by mutations in the CFTR gene. Mutations in the CFTR can also cause cystic fibrosis (CF), an autosomal recessive disease associated with a variety of disorders, including pulmonary and pancreatic dysfunction. Approximately 1 in 25 Caucasians carry a mutation in CFTR. Workup for CBAVD reveals that the male is a compound heterozygote, carrying AF508, the most common mutation in the CFTR gene, and another mutation R117H. Testing of the wife reveals that she also carries the AF508 mutation. Homozygosity for AF508 leads to classic cystic fibrosis. This couple opts to have PGD as part of their assisted reproduction to reduce the chances of having a pregnancy affected by CF. The couple chooses RCNAD as they also wish to reduce their chances of having a pregnancy with a large genomic imbalance. The CFTR gene can be expressed in the blastocyst and can plays a role in formation of the blastocoel.

### Methods

The methods for embryo generation and sampling and RCNAD are performed as outlined in Example 4. For mutation screening, the coding sequences of the CFTR transcripts are examined in detail, looking for presence of the 2 mutations found in the parents: c.1521_1523delCTT, a 3 basepair mutation in exon 11 that causes the AF508 mutation and c.305G>A in exon 4, a single basepair transition that causes the R117H mutation in the CFTR protein. The CFTR transcribed sequences are scanned for other alterations in the CFTR transcript as well. The CFTR transcript sequences are also evaluated for sequence variants and calls are made using the genome analysis toolkit. Five blastocysts are biopsied and cryopreserved.

### Expected Results

As presented in Table III, CFTR mutation analysis reveals 1 embryo to be homozygous for the AF508 mutation, 2 embryos to be compound heterozygotes for the AF508 and R117H mutations and 2 embryos to be carriers of the R117H mutation (WT denotes allele without a mutation). LECNAD and AECNAD reveal that the AF508 homozygote also carries a maternally derived monosomy 1 and R117H carrier (embryo 2) has evidence for triploidy. The finding of that the triploidy has an extra copy of the paternal haploid genome suggests that this triploidy most likely is a result of fertilization by 2 sperm (i.e., dispermy).

TABLE IV

| Test | Emb 1 | Emb 2 | Emb 3 | Emb 4 | Emb 5 |
|---|---|---|---|---|---|
| CFTR | ΔF508 | WT R117H | ΔF508 | ΔF508 | WT R117H |
| | R117H | | R117H | ΔF508 | |
| LECNAD | No CNA | +All chrom | No CNA | −1 | No CNA |
| AECNAD | No imb | ↑All Chrom | No CNA | ↑1 | No imb |
| (P:M) | | | | | |
| BICNAD | None | None | None | None | None |
| SECNAD | Low | High | Low | Low | Low |
| Plan | Res | Res | Res | Res | Tfer |

Based on these results, a decision is made by the healthcare team and parents to transfer embryo 5, which carries the R117H mutation and has no evidence of CNAs.

### XI.H. Example 8

### RCNAD and Linkage Analysis

In this prophetic example, an African-American couple who are both carriers of the sickle cell mutation (HbSS mutation) decide to use ART & PGD to prevent having a pregnancy affected with sickle cell disease, an autosomal recessive disorder that is characterized by intermittent vaso-occlusive events and chronic hemolytic anemia. They have one affected child. In considering options, the couple choose to use transcriptome-based linkage analysis and CNA screening to reduce the risks of establishing a pregnancy affected by sickle cell disease or aneuploidy.

### Methods

The methods for embryo generation and sampling and RCNAD are performed as outlined in Example 4. The haplotypes of the parents and the affected child are first determined by genotyping these individuals. Genomic DNA is isolated from peripheral blood samples using the QIAmp DNA mini blood kit (Qiagen). The individuals are genotyped using an Affymetrix SNP 6.0 microarray. The haplotypes for the three individuals are generated using Triocaller software (Chen, et al. (2013) Genome Research 23: 142-151, incorporated herein by reference). Embryos are screened for CNAs as described in Example 2. SNP genotype data are generated using the genome analysis toolkit. Multipoint linkage analysis for the parents and embryos is performed using SNPLINK software (Webb, et al. (2005) Bioinformatics 21: 3060-3061, incorporated by reference herein)

### Expected Results

Haplotype analysis identifies multiple informative SNPs that are closely linked to the HbSS alleles in both parents. Six embryos are biopsied and cryopreserved. Linkage analysis reveals that two are HbSS homozygotes, 3 are HbSS heterozygotes and 1 is homozygous unaffected. LECNAD and AECNAD reveal that one of the HbSS heterozygotes has evidence for trisomy 7 and the unaffected embryo has evidence for trisomy 18. No breakpoints are identified. SECNAD finds that the 2 trisomies are supported by high risk profiles. Embryo 6, which has no evidence of a CNA is found to have a high risk trisomy profile, which indicates a poor chance of pregnancy based on clinical data. The results are conveyed to the healthcare provider.

TABLE V

| Test | Emb 1 | Emb 2 | Emb 3 | Emb 4 | Emb 5 | Emb 6 |
|---|---|---|---|---|---|---|
| HbSS | HbSS | HbSS | WT | HbSS | HbSS | HbSS |
| linkage | WT | HbSS | WT | WT | HbSS | WT |
| LECNAD | No | No | +18 | No | +7 | No |
| | CNA | CNA | | CNA | | CNA |
| AECNAD | No imb | No imb | ↓18 | No imb | ↓7 | No imb |
| (P:M) | | | | | | |
| BICNAD | None | None | None | None | None | None |
| SECNAD | Low | Low | High | Low | High | High |
| Plan | Cryo | Res | Res | Tfer | Res | Res |

Based on these results, a decision is made by the healthcare team and parents to transfer an HbSS carrier embryo without evidence of large CNAs and to maintain the other one in cryo.

### XI.I. Example 9

### RCNAD and Screening for an Imprinting Disorder

In this prophetic example, a couple who are undergoing IVF for fertility treatment are very knowledgeable about the potential adverse outcomes from IVF. They express their wish to screen embryos for large CNAs and for abnormalities in genomic imprinting that are associated with Beckwith Wiedemann syndrome (BWS). BWS is a growth disorder characterized by a number of malformations and an increased risk for embryonal tumors. This disorder arises from an increased expression of loci in 11p15.5 that are normally expressed from the paternal chromosome. Children of subfertile parents conceived by assisted reproductive technology appear to have about a 9-fold increased risk for this disorder.

### Methods

The methods for embryo generation and sampling and RCNAD are performed as outlined in Example 4. For evaluating imprinting of the BWS region, the expression of the parental alleles of 13 loci in the 11p15.5 region including KCNQ1OT1 and CDKN1C are evaluated using allele-specific SNPs. In the normal situation, the paternal haplotype should express KCNQ1OT1 and not any of the neighboring loci whereas the KCNQ1OT1 should not be expressed and all of the neighboring alleles should in the maternal allele. The identification of skewing of AERs in this region consistent with these normal patterns of locus expression can indicate that this chromosomal region is normally imprinted. In cases in which there is overexpression of the loci that are normally expressed from this region following paternally inheritance, there is an increased risk for BWS. Eight embryos are biopsied and cryopreserved.

### Expected Results

All are found to have the normal pattern of allelic expression in the 11p15.5 region associated with BWS, suggesting that the likelihood of BWS developing from these embryos is very low (Table VI). LECNAD and AECNAD identify 4 embryos without evidence for CNAs and the remainder to have maternally derived aneuploidies.

TABLE VI

| Test | Emb 1 | Emb 2 | Emb 3 | Emb 4 | Emb 5 | Emb 6 | Emb 7 | Emb 8 |
|---|---|---|---|---|---|---|---|---|
| 11p15 | Nl | Nl | Nl | Nl | Nl | Nl | Nl | Nl |
| Imprinting | | | | | | | | |
| LECNAD | −5 | +20 | −13 | No | No | −22 | No | No |
| | | | +17 | CNA | CNA | | CNA | CNA |
| AECNAD | ↑ 5 | ↓ 20 | ↑13 | No | No | ↑ 22 | No | No |
| (P:M) | | | ↓17 | imb | imb | | imb | imb |
| BICNAD | None | None | None | None | None | None | None | None |
| SECNAD | Low | High | High | Low | Low | Low | Low | Low |
| Plan | Res | Res | Res | Cryo | Tfer | Res | Cryo | Cryo |

Based on these results, the healthcare team and parents decide to transfer one of the embryos without evidence for a CNA and to cryopreserve the remainder.

### XI.J. Example 10

### RCNAD and Genetic Fingerprinting

In this prophetic example, a couple undergoing IVF opt for RCNAD. During the process of generating embryos, there is concern that sperm from another donor may have been accidentally used. The genetic data from RCNAD is also used to assess paternity.

### Methods

The methods for embryo generation and sampling and RCNAD are performed as outlined in Example 4. Paternity is assessed using the allelic expression ratio data. This analysis looks at thousands of SNPs that are expected to be heterozygous in the event that sperm from the genotyped father was used to generate the embryos. In the event that almost all (>95%, the observed genotyping frequency from the database) w alleles are present with the exception of loss or deletion of a paternal chromosome, these findings can confirm that the intended father is indeed the father. A total of 7 embryos are biopsied and cryopreserved.

### Expected Results

RCNAD finds 3 embryos with evidence for CNAs and 4 without evidence for CNAs (Table VII). Since the allelic ratios are consistent with the locus expression analyses and there is a 97% rate of expected paternal alleles present, these results indicate these embryos are produced by the intended male. RCNAD finds 3 embryos with evidence of aneuploidies.

TABLE VII

| Test | Emb 1 | Emb 2 | Emb 3 | Emb 4 | Emb 5 | Emb 6 | Emb 7 |
|---|---|---|---|---|---|---|---|
| LECNAD | +16 | No | +15 | No | No | −16 | No |
| | | CNA | | CNA | CNA | | CNA |
| AECNAD | ↓ 16 | No | ↓15 | No | No | ↑16 | No |
| (P:M) | | imb | | imb | imb | | imb |
| BICNAD | None | None | None | None | None | None | None |
| SECNAD | High | Low | High | Low | Low | Low | Low |
| Plan | Res | Tfer | Res | Cryo | Cryo | Res | Cryo |

The RCNAD and assessment of paternity are provided to the medical staff. The parents and staff decide to transfer one of the embryos without evidence of a CNA and the other 3 embryos without indications of CNAs are maintained in cryopreservation.

### XI.K. Example 11

### Determination of Embryo Gender

In this prophetic example, a woman who is a carrier of a mutation in the DMD gene, the gene associated with Duchenne muscular dystrophy, wishes to use preimplantation genetic diagnostics to avoid having a boy affected by this X-linked disease. No other relatives are available for linkage analysis. The woman opts to proceed with RCNAD and gender assessment with the goal of establishing a pregnancy with a healthy female fetus.

### Methods

The methods for embryo generation and sampling and RCNAD are performed as outlined in Example 4. To determine the gender of the embryo, the expression profiles of the sex chromosomes are evaluated. First, it is determined if there is expression of Y-linked loci outside of the pseudoautosomal region. Second, the expression of X-linked loci outside of the pseudoautosomal region is evaluated. A gender of male will be assigned to embryos in which there is Y-linked locus expression and X-linked locus expression consistent with a single copy of this chromosome. A female gender will be assigned for embryos in which there is no evidence of Y-linked locus expression and expression levels of X-linked loci are consistent with 2 copies. Furthermore, SNP genotyping will reveal biallelic patterns for SNPs on the X chromosome.

### Expected Results

In this case, 7 blastocysts are biopsied and cryopreserved. RCNAD results in Table VIII. reveal 3 embryos with trisomies. Of the 4 embryos without evidence of a CNA, 2 are female. One of these embryos is transferred and the other is maintained in cryopreservation.

TABLE VIII

| Test | Emb 1 | Emb 2 | Emb 3 | Emb 4 | Emb 5 | Emb 6 | Emb 7 |
|---|---|---|---|---|---|---|---|
| LECNAD | +5 | No | +22 | No | +12 | No | No |
| | | CNA | | CNA | | CNA | CNA |
| AECNAD | ↓ 5 | No | ↓ 22 | No | ↓ 12 | No | No |
| (P:M) | | imb | | imb | | imb | imb |
| BICNAD | None | None | None | None | None | None | None |
| SECNAD | High | Low | High | Low | High | Low | Low |
| X express | 1X | 1X | 2X | 1X | 2X | 2X | 2X |
| Y express | + | + | − | + | − | − | − |
| Plan | Res | Res | Res | Res | Res | Cryo | Tfer |

Based on these results, the parents and staff decide to transfer one female embryo without evidence of CNAs.

### XI.L. Example 12

### RCNAD and Mitochondrial Mutation Analysis

In this prophetic example, a woman who has a mild form of the mitochondrial disease NARP (neurogenic muscle weakness, ataxia, retinitis pigmentosa) wishes to undergo preimplantation genetic analysis to have an unaffected or less severely affected child. Preimplantation diagnostics have shown that even though this mutation in the mitochondrial genome is maternally transmitted, the mutation load between embryos can vary considerably, with some even having no detectable mutation.

### Methods

The methods for embryo generation and sampling and RCNAD are performed as outlined in Example 4. To identify mitochondrial transcripts, reads will be mapped to the human mitochondrial genome using the same algorithms. Sequence variants and read depths will be determined as described in Example 4. The NARP mutation arises from a guanine to thymine transversion at nucleotide position 8993. The read counts for the wild-type and mutant alleles will provide an indication of the degree of mutation in embryonic cells. Seven blastocysts are biopsied and analyzed.

### Expected Results

RCNAD finds 2 embryos with evidence for aneuploidies and 5 without indication of a CNA (Table IX). Evaluation of the % of the NARP mutation in embryonic RNA ranges from 5-84%. Of the embryos without CNAs, the mutational load for NARP is 5, 15, 33, 52 and 84%.

TABLE IX

| Test | Emb 1 | Emb 2 | Emb 3 | Emb 4 | Emb 5 | Emb 6 | Emb 7 |
|---|---|---|---|---|---|---|---|
| LECNAD | +16 | No | No | No | +13 | No | No |
| | | CNA | CNA | CNA | | CNA | CNA |
| AECNAD | ↓ 16 | No | ↓ 22 | No | ↓ 13 | No | No |
| (P:M) | | imb | | imb | | imb | imb |
| BICNAD | None | None | None | None | None | None | None |
| SECNAD | High | Low | Low | Low | High | Low | Low |
| % NARP | 22% | 5% | 52% | 84% | 7% | 33% | 15% |
| Plan | Res | Tfer | Res | Res | Res | Cryo | Cryo |

Based on these results, the parents and medical team decide to transfer the embryo with no evidence of CNAs and the lowest mutation burden (embryo 2). Other embryos with % NARP <50% and no evidence of a CNA are cryopreserved.

### XI.M. Example 13

### RCNAD and Developmental Potential Assessment

In this prophetic example, an infertile couple wishing to maximize the possibility for having a healthy child produced by IVF opts for RCNAD and assessment of developmental potential.

### Methods

The methods for embryo generation, sampling and RCNAD are performed as outlined in Example 4. For assessment of health and developmental potential, a dataset of transcriptome profiles from embryos that have no evidence of CNAs and are confirmed to produce healthy children is developed using an approach similar to those previously described for developing signature expression profiles. A scoring system is also developed and clinically validated that ranks embryos as low, medium or high developmental potential. Six blastocysts are biopsied and cryopreserved.

### Expected Results

RCNAD analyses find evidence for aneuploidies in 3 embryos and a segmental aneusomy in one (Table X). Of note, the segmental deletion appears to affect the paternal chromosome. Comparisons of the transcriptome profiles for the two embryos without evidence for CNAs find one to have a high developmental potential and one to have a moderate developmental potential.

TABLE X

| Test | Emb 1 | Emb 2 | Emb 3 | Emb 4 | Emb 5 | Emb 6 |
|---|---|---|---|---|---|---|
| LECNAD | No | No | −6q | +11 | +15 | +21 |
| | CNA | CNA | | | | |
| AECNAD | No | No | ↓6q | ↓ 11 | ↓ 15 | ↓ 21 |
| (P:M) | imb | imb | | | | |
| BICNAD | None | None | None | None | None | None |
| SECNAD | Low | Low | Low | High | High | High |
| Dev potent | Mod | High | Mod | Low | Low | Low |
| Plan | Cryo | Tfer | Res | Res | Res | Res |

Based on these results, a decision is made by the healthcare team and parents decide to transfer the embryo without evidence of CNAs and a developmental potential profile consistent with a high developmental potential (embryo 2). The other embryo without signs of a CNA and a moderate developmental potential is maintained in cryopreservation. Embryos with signs of aneuploidy or segmental aneusomy are donated to research.

### XI.N. Example 14

### RCNAD Combined with Other Embryo Diagnostics

In this prophetic example, an infertile couple is interested in using all available modalities for screening their embryos to provide the greatest chance of producing a healthy pregnancy from their IVF cycle. With that goal, the couple decides to have their embryos biopsied to perform RCNAD, mutational screening, genomic imprinting and developmental competence assessment. In addition, noninvasive diagnostics of time-lapsed imaging of embryos and metabolomic and proteomic profiling of culture medium are to be performed. This multifaceted assessment will provide a tremendous amount of information about the health and developmental potential of the embryos.

### Methods

RCNAD is performed as described in Example 4. Mutational screening is an extension of the method described in Example 7 in which the coding regions of loci with sufficient coverage and good allelic representation and identified clinical significance (e.g., loci selected by Kingsmore et al ((2012) PLOS Curr e4f9877) are evaluated for mutations that have either been recognized to be associated with a clinical phenotype or to be predicted to impair the function of the locus. Imprinting analysis as described in Example 6 is extended to evaluate all clinically significant imprinted regions including Beckwith-Wiedemann syndrome and Angelman syndrome regions. Developmental potential assessment is performed as described in Example 13. Metabolic profiling is performed through quantitative analysis of metabolites using ultramicrofluorescent assays for assessing consumption of glucose and pyruvate and production of lactate combined with HPLC for evaluating consumption/production of amino acids (Guerif et al (2013) PLOS One 8: E67834, incorporated herein by reference). Proteomic profiling is performed using nano-ultra-high pressure chromatography and identification via tandem nano-electrospray ionization mass spectrometry with data-independent scanning in a hydrid QqTOF mass spectrometer (Cortezzi et al (2011) Analyt Biochem 401: 1331-9, incorporated herein by reference). Time lapse imaging is performed using the Eeva time-lapse imaging system (Auxogyn, Inc, Conaghan et al (2013) Fert Steril 100: 412-9, incorporated herein by reference). This system analyzes cell division timing data for parameters that have been correlated with successful preimplantation development. For each of these analyses a developmental competence score is assigned that reflects the likelihood of a poor versus good outcome.

### Expected Results

5 embryos are biopsied and analyzed. RCNAD finds two with trisomies, which are supported by SECNAD results. Of the 3 embryos without evidence for CNAs, two have high developmental potential based on the transcriptome profile and the other noninvasive analyses. Two embryos carry the common CF mutation. One embryo with no evidence of a CNA has a moderate developmental potential transcriptome profile and characteristics of poor developmental outcome based on time-lapse imaging.

TABLE XI

| Test | Emb 1 | Emb 2 | Emb 3 | Emb 4 | Emb 5 |
|---|---|---|---|---|---|
| LECNAD | +22 | No CNA | No CNA | No CNA | +10 |
| AECNAD | ↓ 22 | No imb | No imb | No imb | ↓ 22 |
| (P:M) | | | | | |
| BICNAD | None | None | None | None | None |
| SECNAD | High | Low | Low | Low | High |
| Dev Potent | Low | High | Mod | High | Low |
| Mutation | CF | None | None | CF ΔF508/+ | None |
| Screening | ΔF508/+ | | | | |
| Imprinting | Nl | Nl | Nl | Nl | Nl |
| Time Lapse | Poor | Good | Poor | Good | Poor |
| Metabolic | Poor | Good | Good | Good | Poor |
| Proteomic | Poor | Good | Good | Good | Poor |
| Plan | Res | Tfer | Cryo | Cryo | Res |

Based on these results, the healthcare team and parents decide to transfer one of the two embryos without evidence of a CNA and high overall developmental competence scores. The other two embryos without CNAs are maintained in cryopreservation with the embryo with high developmental scores being the next in line for transfer should a subsequent transfer be desired. The two embryos with CNAs are donated to research.

While preferred embodiments have been shown and described herein, it will be obvious to those skilled in the art that such embodiments are provided by way of example only. Numerous variations, changes, and substitutions will now occur to those skilled in the art. It should be understood that various alternatives to the embodiments of the disclosure described herein may be employed. It is intended that the following claims define the scope of the invention and that methods and structures within the scope of these claims and their equivalents be covered thereby.

What is claimed is:

1. A method of determining a presence or absence of a genomic copy number alteration in a preimplantation embryo, the method comprising analyzing RNA from the preimplantation embryo, or cDNA generated from RNA from the preimplantation embryo, to determine the presence or absence of the genomic copy number alteration in the preimplantation embryo.

2. (canceled)

3. The method of claim 1, wherein the analyzing comprises generating sequence data for the RNA or the cDNA, or amplified products thereof, by high-throughput sequencing, whole transcriptome sequencing or partial transcriptome sequencing.

4.-10. (canceled)

11. The method of claim 3, wherein the analyzing comprises comparing an abundance of the sequence reads corresponding to one or more regions on a first chromosome to an abundance of sequence reads corresponding to one or more regions on a second chromosome.

12.-14. (canceled)

15. The method of claim 11, wherein the first and second chromosomes are from the same cell or same embryo.

16.-33. (canceled)

34. The method of claim 1, wherein the RNA is from a plurality of preimplantation embryos, or the cDNA is generated from RNA from a plurality of preimplantation embryos.

35.-39. (canceled)

40. The method of claim 1, wherein the analyzing comprises comparing an amount of RNA or cDNA, or amplified products thereof, derived from one or more regions to an amount of RNA or cDNA derived from the one or more regions from one or more embryos of known copy number for the one or more regions.

41. The method of claim 1, wherein the analyzing comprises comparing an amount of RNA or cDNA, or amplified products thereof, derived from one or more regions to a median expression value.

42.-43. (canceled)

44. The method of claim 1, wherein the analyzing comprises comparing an amount of RNA or cDNA derived from one or more regions to a median expression value of RNA or cDNA derived from the one or more regions from a plurality of embryos.

45.-47. (canceled)

48. The method of claim 1, wherein the analyzing comprises determining a first ratio of an amount of RNA or cDNA derived from a first set of one or more regions to an amount of RNA or cDNA derived from a second set of one or more regions, and comparing the first ratio to a second ratio derived from one or more embryos, wherein the second ratio is a ratio of an amount of RNA or cDNA derived from the first set of one or more regions to an amount of RNA or cDNA derived the second set of one or more regions.

49.-56. (canceled)

57. The method of claim 1, wherein the determining the presence or absence of a copy number alteration comprises use of an algorithm.

58.-60. (canceled)

61. The method of claim 1, wherein the analyzing comprises identifying one or more breakpoints associated with a copy number alteration, wherein the breakpoints are identified by breakpoint sequence in massively parallel sequencing data by identifying split reads or by flanking sequences.

62.-97. (canceled)

98. The method of claim 1, wherein the preimplantation embryo is in a preimplantation period, wherein the preimplantation period encompasses a period that begins with fertilization and extends to a latest timepoint at which an embryo can be maintained in vitro and still produce a healthy liveborn following transfer to a female.

99. (canceled)

100. The method of claim 1, wherein the determining a presence or absence of a copy number alteration in the preimplantation embryo correlates with preimplantation embryonic health or developmental potential.

101. (canceled)

102. The method of claim 1, wherein the analyzing the RNA or cDNA comprises determining regional expression of the RNA or cDNA, identifying breakpoint sequence, and/or detecting a signature expression profile associated with a copy number alteration.

103. The method of claim 1, further comprising analyzing the epigenetic status of the genome of the preimplantation embryo.

104.-106. (canceled)

107. The method of claim 1, further comprising analyzing the RNA or cDNA to determine expression patterns of regions associated with one or more responses to environmental stress, wherein the stress comprises exposure to a toxin, a mutagen, light, high or low temperature, high or low oxygen, oxidative stress, high or low osmolarity, mechanical insult, suboptimal culture conditions or inadequate nutrition.

108. (canceled)

109. The method of claim 1, further comprising analyzing the RNA or cDNA to determine expression patterns of regions associated with metabolism.

110.-112. (canceled)

113. The method of claim 1, wherein the analyzing comprises analyzing expression of one or more RNAs or cDNAs, wherein the analyzing comprises analyzing the expression of one or more genomic regions, wherein the analyzing comprises analyzing expression of one or more loci wherein an expression level of the one or more loci correlates with embryonic health or developmental potential of the preimplantation embryo, or wherein the analyzing comprises analyzing expression of one or more alleles.

114.-119. (canceled)

120. The method of claim 1, wherein the copy number alteration is an aneuploidy.

121.-132. (canceled)

133. The method of claim 1, wherein the determining the presence or absence of the genomic copy number alteration comprises determining an abundance of RNA or cDNA in one or more pre-defined regions of a transcriptome or genome to generate one or more regional expression counts, and the pre-defined region is selected from the group consisting of: an exon, a gene, an allele, a locus, a transcriptional unit or a region of defined length of the transcriptome or genome.

134. (canceled)

135. The method of claim 1, wherein the determining the presence or absence of the genomic copy number alteration in a sample comprises using one or more algorithms to compare one or more regional expression counts from a sample to a reference.

136. (canceled)

137. The method of claim 135, wherein the reference comprises one or more regional expression counts, wherein the reference is generated from one preimplantation embryo, from more than ten preimplantation embryos, from more than 100 preimplantation embryos, or from more than 1000 preimplantation embryos.

138.-145. (canceled)

146. The method of claim 135, wherein the regional expression count is determined by sequencing.

147.-166. (canceled)
